(19) United States
(12) Patent Application Publication
(10) Pub. No.: US 2024/0409587 A1
(43) Pub. Date: Dec. 12, 2024

(54) Coronavirus T Cell Epitopes and Uses Thereof

(71) Applicant: La Jolla Institute for Immunology, La Jolla, CA (US)

(72) Inventors: Alessandro Sette, La Jolla, CA (US); Shane Crotty, La Jolla, CA (US); Grifoni Alba, La Jolla, CA (US); Daniela Weiskopf, La Jolla, CA (US); Bjoern Peters, La Jolla, CA (US)

(21) Appl. No.: 18/553,629

(22) PCT Filed: Apr. 11, 2022
(86) PCT No.: PCT/US2022/024256

§ 371 Date: Aug. 22, 2024

Related U.S. Application Data
(60) Provisional application No. 63/173,634, filed on Apr. 12, 2021.

(51) Int. Cl. C07K 14/005; A61K 39/00; A61P 31/14; C07K 14/74; G01N 33/50; G01N 33/569

(52) U.S. Cl. CPC C07K 14/005; A61P 31/14; C07K 14/70539; G01N 33/505; G01N 33/56983; A61K 2039/55516; A61K 2039/55527; A61K 2039/572; A61K 2039/575; C12N 2770/20022; C12N 2770/20034

(57) ABSTRACT

The present disclosure includes compositions and methods for detecting the presence of: a coronavirus or an immune response relevant to a coronavirus infection including T cells responsive to one or more coronavirus peptides or proteins comprising, consisting of, or consisting essentially of: one or more amino acid sequences selected from those sequences set forth in Table 2 (SEQ ID NOS: 1 to 292), Table 3 (SEQ ID NOS: 293 to 347), or both, or a subsequence, portion, homologue, variant or derivative thereof; a fusion protein; a pool of 2 or more peptides; or a polynucleotide that encodes one or more peptides or proteins, comprising, consisting of, or consisting essentially of one or more amino acid sequences set forth in Table 2 or Table 3, or a subsequence, portion, homologue, variant or derivative thereof. The disclosure further provides vaccines, diagnostics, therapies, and kits, comprising such proteins or peptides.

### CROSS-REFERENCE TO RELATED APPLICATIONS

This application claims priority to U.S. Provisional Application Ser. No. 63/173,634, filed Apr. 12, 2021, the entire contents of which are incorporated herein by reference.

### STATEMENT OF FEDERALLY FUNDED RESEARCH

This invention was made with government support under grant and contract numbers U19 AI142742, 75N9301900065, 75N93019C00001, awarded by the National Institutes of Health/NIAID. The government has certain rights in the invention.

### TECHNICAL FIELD OF THE INVENTION

The present invention relates in general to the field of peptides that are T cell epitopes for coronavirus, and more particularly, to compositions and methods for the prevention, treatment, diagnosis, kits, and uses of such T cell epitopes.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on ______, 2022, is named ______.txt and is ___,___ bytes in size.

### BACKGROUND OF THE INVENTION

Without limiting the scope of the invention, its background is described in connection with coronaviruses.

As of April 2022, SARS-CoV-2 infections are associated with more than 6.15 million deaths and over 491 million cases worldwide, and over 80 million cases in the United States alone (coronavirus.jhu.edu/map.html). The severity of the associated Coronavirus Disease 2019 (COVID-19) ranges from asymptomatic or mild self-limiting disease, to severe pneumonia and acute respiratory distress syndrome (WHO; www.who.int/publications/i/item/clinical-management-of-covid-19). The present inventors and others have started to delineate the role of SARS-CoV-2-specific T cell immunity in COVID-19 clinical outcomes (Altmann and Boyton, 2020; Braun et al., 2020; Grifoni et al., 2020; Le Bert et al., 2020; Meckiff et al., 2020; Rydyznski Moderbacher et al., 2020; Sekine et al., 2020; Weiskopf et al., 2020). A growing body of evidence points to a key role for SARS-CoV-2-specific T cell responses in COVID-19 disease resolution and modulation of disease severity (Rydyznski Moderbacher et al., 2020; Schub et al., 2020; Weiskopf et al., 2020). Milder cases of acute COVID-19 were associated with coordinated antibody, CD4+ and CD8+ T cell responses, whereas severe cases correlated with a lack of coordination of cellular and antibody responses, and delayed kinetics of adaptive responses (Rydyznski Moderbacher et al., 2020; Weiskopf et al., 2020). Now, the emergence of SARS-CoV-2 variants highlights the better need to better understand adaptive immune responses to this virus.

Despite these advances, and in light of variants of SARS-CoV-2 being identified globally, a need remains for identifying T cell epitopes for use in diagnostics, treatments, vaccines, kits, etc.

### SUMMARY OF THE INVENTION

In one embodiment, the present disclosure includes a composition comprising: one or more peptides or proteins, comprising, consisting of, or consisting essentially of an amino acid sequence selected from the sequences set forth in Table 2 (SEQ ID NOS: 1 to 292), Table 3 (SEQ ID NOS: 293 to 347), or both, or a subsequence, portion, homologue, variant or derivative thereof; a fusion protein comprising one or more amino acid sequences selected from those sequences set forth in Table 2, Table 3, or both; a pool of 2 or more peptides comprising, consisting of, or consisting essentially of amino acid sequences selected from those sequences set forth in Table 2, Table 3, or both; or a polynucleotide that encodes one or more peptides or proteins, comprising, consisting of, or consisting essentially of an amino acid sequence selected from those sequences set forth in Table 2, Table 3, or both, or a subsequence, portion, homologue, variant or derivative thereof. In one aspect, the one or more peptides or proteins comprises, or wherein the fusion protein comprises 2 or more or more amino acid sequences selected from those sequences set forth in Table 2, Table 3, or both, or a subsequence, portion, homologue, variant or derivative thereof. In another aspect, the amino acid sequence is selected from a coronavirus T cell epitope selected from those sequences set forth in Table 2. In another aspect, the composition comprises one or more SARS-CoV-2 peptides amino acid sequences selected from those sequences set forth in Table 3, or a subsequence, portion, homologue, variant or derivative thereof; a fusion protein comprising one or more amino acid sequences selected from those sequences set forth in Table 3; or a pool of 2 or more peptides selected from those sequences set forth in Table 3. In another aspect, the peptide or protein comprises a coronavirus T cell epitope. In another aspect, the one or more peptides or proteins comprises a coronavirus CD8+ or CD4+ T cell epitope. In another aspect, the coronavirus is SARS-CoV-2 and the SARS-CoV-2 T cell epitope is not conserved in another coronavirus. In another aspect, the coronavirus is SARS-CoV-2 and the SARS-CoV-2 T cell epitope is conserved in another coronavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases, or enhances a T cell response to a coronavirus. In another aspect, the one or more peptides or proteins that elicits, stimulates, induces, promotes, increases or enhances the T cell response to the coronavirus is a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present disclosure includes a composition comprising monomers or multimers of: peptides or proteins comprising, consisting of, or consisting essentially of: one or more amino acid sequences selected from those sequences set forth in Table 2 (SEQ ID NOS: 1 to 292), Table 3 (SEQ ID NOS: 293 to 347), or both, concatemers, subsequences, portions, homologues, variants or derivatives thereof; a fusion protein comprising one or more amino acid sequences selected from those sequences set forth in Table 2, Table 3, or both; or a polynucleotide that encodes one or more peptides or proteins, comprising, consisting of, or consisting essentially of an amino acid sequence selected from those sequences set forth in Table 2, Table 3, or both, or a subsequence, portion, homologue, variant or derivative thereof.

In another embodiment, the present disclosure includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Table 2 (SEQ ID NOS: 1 to 292), Table 3 (SEQ ID NOS: 293 to 347), or both, in a groove of the MHC monomer or multimer.

In another embodiment, the present disclosure includes a composition comprising: one or more peptides or proteins comprising, consisting of, or consisting essentially of an amino acid sequence selected from those sequences set forth in Table 3 (SEQ ID NOS: 293 to 347), or a subsequence, portion, homologue, variant or derivative thereof; a fusion protein comprising one or more amino acid sequences selected from those sequences set forth in Table 3; a pool of 2 or more peptides selected from those sequences set forth in Table 3; a polynucleotide that encodes one or more peptides or proteins, comprising, consisting of, or consisting essentially of an amino acid sequence selected from those sequences set forth in Table 3, or a subsequence, portion, homologue, variant or derivative thereof. In one aspect, the one or more peptides or proteins comprises, or wherein the fusion protein comprises, 2 or more amino acid sequences selected from those sequences set forth in Table 3, or a subsequence, portion, homologue, variant or derivative thereof. In another aspect, the protein or peptide comprises a SARS-CoV-2 T cell epitope. In another aspect, the one or more peptides or proteins comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to SARS-CoV-2. In another aspect, the one or more peptides or proteins that elicits, stimulates, induces, promotes, increases or enhances the T cell response to SARS-CoV-2 is a SARS-CoV-2 spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands. In another aspect, the composition further comprises a modulator of immune response. In another aspect, the modulator of immune response is a modulator of the innate immune response. In another aspect, the modulator is Interleukin-6 (IL-6), Interferon-gamma (IFN-γ), Transforming growth factor beta (TGF-β), or Interleukin-10 (IL-10), or an agonist or antagonist thereof.

In another embodiment, the present disclosure includes a composition comprising monomers or multimers of: one or more peptides or proteins comprising, consisting of, or consisting essentially of: one or more SARS-CoV-2 amino acid sequences selected from those sequences set forth in Table 3 (SEQ ID NOS: 293 to 347), concatemers, subsequences, portions, homologues, variants or derivatives thereof; a fusion protein comprising one or more amino acid sequences selected from those sequences set forth in Table 3; or a polynucleotide that encodes one or more peptides or proteins, comprising, consisting of, or consisting essentially of an amino acid sequence selected from those sequences set forth in Table 3, or a subsequence, portion, homologue, variant or derivative thereof.

In another embodiment, the present disclosure includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Table 3 (SEQ ID NOS: 293 to 347), in a groove of the (MHC) monomer or multimer.

In another embodiment, the present disclosure includes a method for detecting the presence of: (i) a coronavirus or (ii) an immune response relevant to coronavirus infections, vaccines or therapies, including T cells responsive to one or more coronavirus peptides, comprising: providing one or more proteins or peptides for detection of an amount or a relative amount of, and/or the activity of, and/or the state of antigen-specific T-cells; contacting a biological sample suspected of having coronavirus-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in Table 2 (SEQ ID NOS: 1 to 292), Table 3 (SEQ ID NOS: 293 to 347), or both, or comprise a pool of 2 or more amino acid sequences set forth in Table 2, Table 3, or both. In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from Table 2 (SEQ ID NOS: 1 to 292). In another aspect, the detecting the amount or a relative amount of, and/or activity of antigen-specific T-cells comprises indirect detection and/or direct detection. In another aspect, the method of detecting an immune response relevant to the coronavirus comprises the following steps: providing an MHC monomer or an MHC multimer; contacting a population T-cells to the MHC monomer or MHC multimer; and measuring the number, activity or state of T-cells specific for the MHC monomer or MHC multimer. In one aspect, the MHC monomer or MHC multimer comprises a protein or peptide of the coronavirus. In another aspect, the protein or peptide comprises a CD8+ or CD4+ T cell epitope. In another aspect, the T cell epitope is not conserved in another coronavirus. In another aspect, the T cell epitope is conserved in another coronavirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from those sequences set forth in Table 2 (SEQ ID NOS: 1 to 292), Table 3 (SEQ ID NOS: 293 to 347), or both, or a subsequence, portion, homologue, variant or derivative thereof. In another aspect, the method further comprises detecting the presence or amount of the one or more peptides in a biological sample, or a response thereto, which is diagnostic of a coronavirus infection. In another aspect, the detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present disclosure includes a method for detecting the presence of: (i) SARS-CoV-2 or (ii) an immune response relevant to SARS-CoV-2 infections, vaccines or therapies, including T cells responsive to one or more SARS-CoV-2 peptides, comprising: providing one or more proteins or peptides for detection of an amount or a relative amount of, and/or the activity of, and/or the state of antigen-specific T-cells; contacting a biological sample suspected of having SARS-CoV-2-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in those sequences set forth in Table 3 (SEQ ID NOS: 293 to 347), or comprise a pool of 2 or more amino acid sequences set forth in those sequences set forth in Table 3. In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from those sequences set forth in Table 3. In another aspect, detecting the amount or a relative amount of, and/or activity of antigen-specific T-cells comprises indirect detection and/or direct detection. In another aspect, detecting an immune response relevant to SARS-CoV-2 comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, the MHC monomer or MHC multimer comprises a protein or peptide of SARS-CoV-2. In another aspect, the protein or peptide comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from those sequences set forth in Table 3, or a subsequence, portion, homologue, variant or derivative thereof. In another aspect, the method further comprises detecting the presence or amount of the one or more peptides in a biological sample, or a response thereto, which is diagnostic of a SARS-CoV-2 infection. In another aspect, detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present disclosure includes a method detecting a coronavirus infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with coronavirus. In one aspect, the sample comprises T cells. In another aspect, the response comprises inducing, increasing, promoting or stimulating anti-coronavirus activity of T cells. In another aspect, the T cells are CD8+ or CD4+ T cells. In another aspect, the method comprises determining whether the subject has been infected by or exposed to the coronavirus more than once by determining if the subject elicits a secondary T cell immune response profile that is different from a primary T cell immune response profile. In another aspect, the method further comprises diagnosing a coronavirus infection or exposure in a subject, the method comprising contacting a biological sample from a subject with a composition of composition of one or more proteins, peptides or multimers, and determining if the composition elicits a T cell immune response, wherein the T cell immune response identifies that the subject has been infected with or exposed to a coronavirus. In another aspect, the method is conducted three or more days following the date of suspected infection by or exposure to a coronavirus.

In another embodiment, the present disclosure includes a method detecting SARS-CoV-2 infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with SARS-CoV-2. In another aspect, the sample comprises T cells. In another aspect, the response comprises inducing, increasing, promoting or stimulating anti-SARS-CoV-2 activity of T cells. In another aspect, the T cells are CD8+ or CD4+ T cells. In another aspect, the method comprises determining whether the subject has been infected by or exposed to SARS-CoV-2 more than once by determining if the subject elicits a secondary T cell immune response profile that is different from a primary T cell immune response profile. In another aspect, the method further comprises diagnosing a SARS-CoV-2 infection or exposure in a subject, the method comprising contacting a biological sample from a subject with a composition of one or more proteins, peptides or multimers; and determining if the composition elicits a T cell immune response, wherein the T cell immune response identifies that the subject has been infected with or exposed to SARS-CoV-2. In another aspect, the method is conducted three or more days following the date of suspected infection by or exposure to a coronavirus.

In another embodiment, the present disclosure includes a kit for the detection of coronavirus or an immune response to coronavirus in a subject comprising, consisting of or consisting essentially of: one or more T cells that specifically detect the presence of: one or more amino acid sequences selected from those sequences set forth in Table 2 (SEQ ID NOS: 1 to 292), Table 3 (SEQ ID NOS: 293 to 347), or both, or a subsequence, portion, homologue, variant or derivative thereof; or a fusion protein comprising one or more amino acid sequences selected from those sequences set forth in Table 2, Table 3, or both; or a pool of 2 or more peptides selected from the amino acid sequences set forth in Table 2, Table 3, or both. In one aspect, the one or more amino acid sequences are selected from a coronavirus T cell epitope set forth in Table 2. In another aspect, the composition comprises: one or more amino acid sequences selected from those sequences set forth in Table 3, or a subsequence, portion, homologue, variant or derivative thereof; a fusion protein comprising one or more amino acid sequences selected from those sequences set forth in Table 3; or a pool of 2 or more peptides selected from the amino acid sequences set forth in those sequences set forth in Table 3. In another aspect, the amino acid sequence comprises a coronavirus CD8+ or CD4+ T cell epitope. In another aspect, the T cell epitope is not conserved in another coronavirus. In another aspect, the T cell epitope is conserved in another coronavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) coronavirus or (ii) an immune response relevant to coronavirus infections, vaccines or therapies, including T cells responsive to coronavirus. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to coronavirus.

In another embodiment, the present disclosure includes a kit for the detection of SARS-CoV-2 or an immune response to SARS-CoV-2 in a subject comprising, consisting of or consisting essentially of: one or more T cells that specifically detect the presence of: one or more amino acid sequences selected from those sequences set forth in Table 3 (SEQ ID NOS: 293 to 347), or a subsequence, portion, homologue, variant or derivative thereof; a fusion protein comprising one or more amino acid sequences selected from those sequences set forth in Table 3; or a pool of 2 or more peptides selected from the amino acid sequences set forth in those sequences set forth in Table 3. In another aspect, the amino acid sequence comprises a SARS-CoV-2 CD8+ or CD4+ T cell epitope. In another aspect, the SARS-CoV-2 T cell epitope is not conserved in another coronavirus. In another aspect, the SARS-CoV-2 T cell epitope is conserved in another coronavirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) SARS-CoV-2 or (ii) an immune response relevant to SARS-CoV-2 infections, vaccines or therapies, including T cells responsive to SARS-CoV-2. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to SARS-CoV-2.

In another embodiment, the present disclosure includes a method of stimulating, inducing, promoting, increasing, or enhancing an immune response against a coronavirus in a subject, comprising: administering a composition of one or more proteins, peptides, multimers or a polynucleotide that expresses the protein, peptide or multimers, in an amount sufficient to stimulate, induce, promote, increase, or enhance an immune response against the coronavirus in the subject. In another aspect, the immune response provides the subject with protection against a coronavirus infection or pathology, or one or more physiological conditions, disorders, illnesses, diseases or symptoms caused by or associated with coronavirus infection or pathology. In another aspect, the immune response is specific to: one or more SARS-CoV-2 peptides selected from the amino acid sequences set forth in Table 2 (SEQ ID NOS: 1 to 292) or Table 3 (SEQ ID NOS: 293 to 347), or a subsequence, portion, homologue, variant or derivative thereof.

In another embodiment, the present disclosure includes a method of stimulating, inducing, promoting, increasing, or enhancing an immune response against SARS-CoV-2 in a subject, comprising: administering a composition of proteins, peptides, multimers or a polynucleotide that expresses the protein, peptide or multimers, in an amount sufficient to stimulate, induce, promote, increase, or enhance an immune response against SARS-CoV-2 in the subject. In one aspect, the immune response provides the subject with protection against a SARS-CoV-2 infection or pathology, or one or more physiological conditions, disorders, illnesses, diseases or symptoms caused by or associated with SARS-CoV-2 infection or pathology. In another aspect, the immune response is specific to: one or more SARS-CoV-2 peptides selected from the amino acid sequences set forth in those sequences set forth in Table 3 (SEQ ID NOS: 293 to 347), or a subsequence, portion, homologue, variant or derivative thereof.

In another embodiment, the present disclosure includes a method of stimulating, inducing, promoting, increasing, or enhancing an immune response against SARS-CoV-2 in a subject, comprising: administering to a subject an amount of a protein or peptide comprising, consisting of or consisting essentially of an amino acid sequence of the SARS-CoV-2 spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two peptides selected from the amino acid sequences set forth in Table 2 (SEQ ID NOS: 1 to 292) or Table 3 (SEQ ID NOS: 293 to 347) or a subsequence, portion, homologue, variant or derivative thereof, in an amount sufficient to prevent, stimulate, induce, promote, increase, immunize against, or enhance an immune response against SARS-CoV-2 in the subject. In one aspect, the immune response provides the subject with protection against SARS-CoV-2 infection or pathology, or one or more physiological conditions, disorders, illnesses, diseases or symptoms caused by or associated with SARS-CoV-2 infection or pathology.

In another embodiment, the present disclosure includes a method of treating, preventing, or immunizing a subject against SARS-CoV-2 infection, comprising administering to a subject an amount of a protein or peptide comprising, consisting of, or consisting essentially of an amino acid sequence of a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two amino acid sequences selected from Table 2 (SEQ ID NOS: 1 to 292), Table 3 (SEQ ID NOS: 293 to 347), or both, or a subsequence, portion, homologue, variant or derivative thereof, in an amount sufficient to treat, prevent, or immunize the subject for SARS-CoV-2 infection, wherein the protein or peptide comprises or consists of a coronavirus T cell epitope that elicits, stimulates, induces, promotes, increases, or enhances an anti-SARS-CoV-2 T cell immune response. In one aspect, the one or more amino acid sequences are selected from those sequences set forth in Table 3, or a subsequence, portion, homologue, variant or derivative thereof; a fusion protein comprising one or more amino acid sequences selected from those sequences set forth in Table 3; or a pool of 2 or more peptides selected from the amino acid sequences set forth in those sequences set forth in Table 3. In one aspect, the anti-SARS-CoV-2 T cell response is a CD8+, a CD4+ T cell response, or both. In another aspect, the T cell epitope is conserved across two or more clinical isolates of SARS-CoV-2, two or more circulating forms of SARS-CoV-2, or two or more coronaviruses. In another aspect, the SARS-CoV-2 infection is an acute infection. In another aspect, the subject is a mammal or a human. In another aspect, the method reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid. In another aspect, the method reduces one or more adverse physiological conditions, disorders, illness, diseases, symptoms or complications caused by or associated with SARS-CoV-2 infection or pathology. In another aspect, the method improves one or more adverse physiological conditions, disorders, illness, diseases, symptoms or complications caused by or associated with SARS-CoV-2 infection or pathology. In another aspect, the symptom is fever or chills, cough, shortness of breath or difficulty breathing, fatigue, muscle or body aches, headache, new loss of taste or smell, sore throat, congestion or runny nose, nausea or vomiting, or diarrhea. In another aspect, the method reduces or inhibits susceptibility to SARS-CoV-2 infection or pathology. In another aspect, the protein or peptide, or a subsequence, portion, homologue, variant or derivative thereof, is administered prior to, substantially contemporaneously with or following exposure to or infection of the subject with SARS-CoV-2. In another aspect, a plurality of SARS-CoV-2 T cell epitopes are administered prior to, substantially contemporaneously with or following exposure to or infection of the subject with SARS-CoV-2. In another aspect, the protein or peptide, or a subsequence, portion, homologue, variant or derivative thereof is administered within 2-72 hours, 2-48 hours, 4-24 hours, 4-18 hours, or 6-12 hours after a symptom of SARS-CoV-2 infection or exposure develops. In another aspect, the protein or peptide, or a subsequence, portion, homologue, variant or derivative thereof is administered prior to exposure to or infection of the subject with SARS-CoV-2. In another aspect, the method further comprises administering a modulator of immune response prior to, substantially contemporaneously with or following the administration to the subject of an amount of a protein or peptide. In another aspect, the modulator of immune response is a modulator of the innate immune response. In another aspect, the modulator is IL-6, IFN-γ, TGF-β, or IL-10, or an agonist or antagonist thereof.

In another embodiment, the present disclosure includes a method of treating, preventing, or immunizing a subject against SARS-CoV-2 infection, comprising administering to a subject the composition of one or more proteins, peptides or multimers in an amount sufficient to treat, prevent, or immunize the subject for SARS-CoV-2 infection. In one aspect, the SARS-CoV-2 infection is an acute infection. In another aspect, the method reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid. In another aspect, the method reduces one or more adverse physiological conditions, disorders, illness, diseases, symptoms or complications caused by or associated with SARS-CoV-2 infection or pathology. In another aspect, the method improves one or more adverse physiological conditions, disorders, illness, diseases, symptoms or complications caused by or associated with SARS-CoV-2 infection or pathology. In another aspect, the symptom is fever or chills, cough, shortness of breath or difficulty breathing, fatigue, muscle or body aches, headache, new loss of taste or smell, sore throat, congestion or runny nose, nausea, vomiting, or diarrhea. In another aspect, the method reduces or inhibits susceptibility to SARS-CoV-2 infection or pathology. In another aspect, the composition is administered prior to, substantially contemporaneously with or following exposure to or infection of the subject with SARS-CoV-2. In another aspect, the composition is administered prior to, substantially contemporaneously with or following exposure to or infection of the subject with SARS-CoV-2. In another aspect, the composition is administered within 2-72 hours, 2-48 hours, 4-24 hours, 4-18 hours, or 6-12 hours after a symptom of SARS-CoV-2 infection or exposure develops. In another aspect, the composition is administered prior to exposure to or infection of the subject with SARS-CoV-2.

In another embodiment, the present disclosure includes a peptide or peptides that are immunoprevalent or immunodominant in a virus obtained by a method consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a coronavirus. In another aspect, the coronavirus is SARS-CoV-2. In another aspect, the immunodominant peptides are selected from 1, 2 or more peptides selected from the amino acid sequences set forth in Table 2 (SEQ ID NOS: 1 to 292) or Table 3 (SEQ ID NOS: 293 to 347). In another aspect, the immunodominant peptides are selected from 1, 2 or more peptides selected from the amino acid sequences set forth in those sequences set forth in Table 3.

In another embodiment, the present disclosure includes a method of selecting an immunoprevalent or immunodominant peptide or protein of a virus comprising, consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a coronavirus. In another aspect, the coronavirus is SARS-CoV-2. In another aspect, the immunodominant peptides are selected from 1, 2 or more peptides selected from the amino acid sequences set forth in Table 2 (SEQ ID NOS: 1 to 292), Table 3 (SEQ ID NOS: 293 to 347), or both. In another aspect, the immunodominant peptides are selected from 1, 2 or more peptides selected from the amino acid sequences set forth in those sequences set forth in Table 3.

In another embodiment, the present disclosure includes a polynucleotide that expresses one or more peptides or proteins, comprising, consisting of, or consisting essentially of an amino acid sequence selected from those sequences set forth in Table 2 (SEQ ID NOS: 1 to 292), Table 3 (SEQ ID NOS: 293 to 347), or both, or a subsequence, portion, homologue, variant or derivative thereof; a fusion protein comprising one or more amino acid sequences selected from those sequences set forth in Table 2, Table 3, or both; or a pool of 2 or more peptides comprising, consisting of, or consisting essentially of amino acid sequences selected from those sequences set forth in Table 2, Table 3, or both. In one aspect, the vector comprises the polynucleotide of claim that expresses one or more peptides or proteins, comprising, consisting of, or consisting essentially of an amino acid sequence selected from those sequences set forth in Table 2, Table 3, or both, or a subsequence, portion, homologue, variant or derivative thereof; a fusion protein comprising one or more amino acid sequences selected from those sequences set forth in Table 2, Table 3, or both; or a pool of 2 or more peptides comprising, consisting of, or consisting essentially of amino acid sequences selected from those sequences set forth in Table 2, Table 3, or both, a viral vector, or a host cell the comprises the same.

In another embodiment, the present disclosure includes a polynucleotide that expresses one or more peptides or proteins comprising, consisting of, or consisting essentially of an amino acid sequence selected from those sequences set forth in Table 3 (SEQ ID NOS: 293 to 347), or a subsequence, portion, homologue, variant or derivative thereof; a fusion protein comprising one or more amino acid sequences selected from those sequences set forth in Table 3; or a pool of 2 or more peptides selected from those sequences set forth in Table 3. In one aspect, the vector comprises the polynucleotide of claim that expresses one or more peptides or proteins comprising, consisting of, or consisting essentially of an amino acid sequence selected from those sequences set forth in Table 3, or a subsequence, portion, homologue, variant or derivative thereof; a fusion protein comprising one or more amino acid sequences selected from those sequences set forth in Table 3; or a pool of 2 or more peptides selected from those sequences set forth in Table 3, a viral vector, or a host cell that comprises the same.

### BRIEF DESCRIPTION OF THE DRAWINGS

For a more complete understanding of the features and advantages of the present invention, reference is now made to the detailed description of the invention along with the accompanying figures and in which:

FIGS. 1A to 1G. T cell responses of COVID-19 convalescent individuals against ancestral and variant SARS-CoV-2 Spike. PBMCs of COVID-19 convalescent individuals (n=28) were stimulated with the Spike MPs corresponding to the ancestral reference strain (Column 1) and the B.1.1.7 (Column 2), B.1.351 (Column 3), P.1 (Column 4) and CAL.20C (Column 5) SARS-CoV-2 variants. FIG. 1A) The gating strategy for the AIM assay is illustrated by representative graphs defining Spike-specific CD4⁺ or CD8⁺ T cells by expression of OX40⁺CD137⁺ and CD69⁺CD137⁺, respectively. These graphs depict one of the COVID-19 convalescent donors from this study tested with the S MPs corresponding to each of the VOCs tested. FIG. 1B) Percentages of AIM⁺ (OX40⁺CD137⁺) CD4⁺ T cells. FIG. 1C) Percentages of AIM⁺ (CD69⁺CD137⁺) CD8⁺ T cells. FIG. 1D) IFNγ spot forming cells (SFC) per million PBMCs FIG. 1E) IL-5 SFC per million PBMCs. Paired comparisons of ancestral S MP versus each of the variants were performed by one-tailed Wilcoxon test and are indicated by the p values in panels FIG. 1B-FIG. 1D. The data shown in panels FIG. 1B and FIG. 1C are plotted to show the Spike MPs titration (1 μg/mL, 0.1 μg/mL, and 0.01 μg/mL) for CD4⁺ (FIG. 1F) and CD8⁺ (FIG. 1G) T cells for each SARS-CoV-2 variant and the geometric mean of the 0.1 μg/mL condition is listed above each titration. In all panels, the bars represent the geometric mean.

FIGS. 2A to 2D. T cell responses of COVID-19 convalescent individuals against ancestral and variant SARS-CoV-2 proteomes. PBMCs of COVID-19 convalescent individuals (n=28) were stimulated with MPs for the entire viral proteome corresponding to the ancestral reference strain (Column 1) and the B.1.1.7 (Column 2), B.1.351 (Column 3), P.1 (Column 4) and CAL.20C (Column 5) SARS-CoV-2 variants. FIG. 2A) Percentages of AIM⁺ (OX40⁺CD137⁺) CD4⁺ T cells for the total reactivity.

FIG. 2B) Percentages of AIM⁺ (CD69⁺CD137⁺) CD8⁺ T cells for the total reactivity. FIG. 2C) Percentages of AIM⁺ (OX40⁺CD137⁺) CD4⁺ T cells for each MP. FIG. 2D) Percentages of AIM⁺ (CD69⁺CD137⁺) CD8⁺ T cells for each MP. All bars represent the geometric mean.

FIGS. 3A to 3H. T cell responses of COVID-19 vaccinees against ancestral and variant SARS-CoV-2 Spike. PBMCs of Pfizer/BioNTech BNT162b2 (n=14, triangles) and Moderna mRNA-1273 COVID-19 vaccines (n=15, circles) were stimulated with the Spike MPs corresponding to the ancestral reference strain (Column 1) and the B.1.1.7 (Column 2), B.1.351 (Column 3), P.1 (Column 4) and CAL.20C (Column 5) SARS-CoV-2 variants. FIG. 3A) The gating strategy for the AIM assay is illustrated by representative graphs defining Spike-specific CD4⁺ or CD8⁺ T cells by the expression of OX40⁺CD137⁺ and CD69⁺CD137⁺, respectively. These graphs depict one of the COVID-19 vaccinated donors from this study tested with the S MPs corresponding to each of the VOCs tested. FIG. 3B) Percentages of AIM⁺ (OX40⁺CD137⁺) CD4⁺ T cells. FIG. 3C) Percentages of AIM⁺ (CD69⁺CD137⁺) CD8⁺ T cells. FIG. 3D) IFNγ spot forming cells (SFC) per million PBMCs FIG. 3E) IL-5 Spot forming cells (SFC) per million PBMCs. FIG. 3F) Percentages of IFNγ were calculated from the total IFNγ and IL-5 SFC per million PBMCs. Paired comparisons of the ancestral reference strain-based S MP versus each of the variants were performed by one-tailed Wilcoxon test and are indicated by the p values in panels FIG. 3B-FIG. 3D. The data shown in panels FIG. 3B and FIG. 3C are also plotted showing the Spike MPs titration (1 μg/mL, 0.1 μg/mL, and 0.01 μg/mL) for CD4⁺ (FIG. 3G) and CD8⁺ (FIG. 3H) T cells with each SARS-CoV-2 variant. The geometric mean of the 0.1 ug/mL condition is listed above each titration. In all panels, the bars represent the geometric mean.

FIGS. 4A to 4M. SARS-CoV-2 T cell epitope sequences affected by the variants. CD4⁺ and CD8⁺ T cell epitopes of the ancestral strain identified in a previous study⁵¹ were analyzed as a function of the number and percentage of response that are or are not conserved across the B.1.1.7 (Column/Row 1), B.1.351 (Column/Row 2), P.1 (Column/Row 3) and CAL.20C (Column/Row 4) SARS-CoV-2 variants. The SARS-CoV-2 epitopes for the most immunodominant SARS-CoV-2 proteins in terms of numbers and percentage of response are shown for CD4⁺ (FIG. 4A-B) and CD8⁺ (FIG. 4C-D) T cells. The SARS-CoV-2 epitopes for the Spike protein only in terms of numbers and percentage of response are shown for CD4⁺ (FIG. 4E-F) and CD8⁺ (FIG. 4G-H) T cells. The peptide binding algorithm data of mutated versus wild type epitopes are shown for CD8⁺ (FIG. 4I-L) as well as the effect of the mutations on CD8⁺ T cell epitopes, where each instance was categorized as a function of whether the binding capability of the mutated peptide was increased (>2-fold), neutral or decreased (<2-fold) (FIG. 4M).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. Numerous variations, changes, and substitutions will now occur to those skilled in the art without departing from the invention. It should be understood that various alternatives to the embodiments of the invention described herein may be employed in practicing the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless specifically stated or obvious from context, as used herein, the term “or” is understood to be inclusive.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular embodiments, a pharmaceutical composition is suitable for specific or non-specific immunotherapy or is a vaccine composition.

Isolated nucleic acid (including isolated nucleic acid) encoding the proteins and peptides are also provided. Cells expressing a protein or peptide are further provided. Such cells include eukaryotic and prokaryotic cells, such as mammalian, insect, fungal and bacterial cells.

Methods and uses and medicaments of proteins and peptides of the invention are included. Such methods, uses and medicaments include modulating immune activity of a cell against a pathogen, for example, a bacteria or virus.

The term “peptide mimetic” or “peptidomimetic” refers to protein-like chain designed to mimic a peptide or protein. Peptide mimetics may be generated by modifying an existing peptide or by designing a compound that mimic peptides, including peptoids and β-peptides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

A “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

As used herein, the terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

As used herein, the term “multimer” refers to a complex comprising multiple monomers (e.g., a protein complex) associated by covalent and/or noncovalent bonds. The monomers can be substantially identical monomers, or the monomers may be different. In embodiments, the multimer is a dimer, a trimer, a tetramer, or a pentamer.

As used herein, the term “Major Histocompatibility Complex” (MHC) is a generic designation meant to encompass the histocompatibility antigen systems described in different species including the human leucocyte antigens (HLA). Typically, MHC Class I or Class II multimers are well known in the art and include but are not limited to dimers, tetramers, pentamers, hexamers, heptamers and octamers.

As used herein, the term “MHC/peptide multimer” refers to a multimeric complex such as a stable multimeric complex composed of or comprising MHC protein(s) subunits loaded with a peptide (MHC/peptide monomers) of the present disclosure. For example, an MHC/peptide multimer (also called herein MHC/peptide complex) include, but are not limited to, an MHC/peptide dimer, trimer, tetramer, pentamer or higher valency multimer, e.g., comprising 2, 3, 4, 5, 6, 7, 8, 9, 10, 11, 12, 13, 14, 15, 16, 17, 18, 19, 20, 21, 22, 23, 24 or more than 24 MHC/peptide monomers. In humans there are three major different genetic loci that encode MHC class I molecules (the MHC molecules of the human are also designated human leukocyte antigens (HLA)): HLA-A, HLA-B, HLA-C, e.g., HLA-A*01, HLA-A*02, and HLA-A*11 are examples of different MHC class I alleles that can be expressed from these loci. Non-classical human MHC class I molecules such as HLA-E (homolog of mice Qa-1b) and MICA/B molecules are also encompassed by the present disclosure. In some embodiments, the MHC/peptide multimer is an HLA/peptide multimer selected from the group consisting of HLA-A/peptide multimer, HLA-B/peptide multimer, HLA-C/peptide multimer, HLA-E/peptide multimer, MICA/peptide multimer and MICB/peptide multimer.

In one embodiment the term “MHC/peptide multimer” refers to a complex comprising multiple MHC/peptide monomers (i.e., at least two MHC/peptide monomers) associated by covalent and/or noncovalent bonds. The MHC/peptide monomers can be substantially identical MHC/peptide monomers, or the MHC/peptide monomers may be different. In one embodiment the MHC/peptide multimer comprises a peptide comprising, consisting of, or consisting essentially of an amino acid sequence selected from those sequences set forth in Table 2 (SEQ ID NOS: 1 to 292) and/or Table 3 (SEQ ID NOS: 293 to 347), preferably in a groove of the MHC monomer. Each MHC/peptide monomer of the MHC/peptide multimer can be associated with one or more multimerization domains such as a multimerization domain selected from the group consisting of IgG, streptavidin, avidin, streptactin, micelles, cells, polymers, dextran, polysaccharides, beads and other types of solid support, and small organic molecules carrying reactive groups or carrying chemical motifs that can bind MHC complexes.

In one embodiment the MHC/peptide multimer comprises at least 2 MHC/peptide monomers, such as at least 3 MHC/peptide monomers such as at least 4 MHC/peptide monomers, such as at least 5 MHC/peptide monomers, such as at least 6 MHC/peptide monomers, such as at least 8 MHC/peptide monomers, such as at least 10 MHC/peptide monomers, such as at least 12 MHC/peptide monomers, such as at least 14 MHC/peptide monomers, such as at least 16 MHC/peptide monomers, such as at least 18 MHC/peptide monomers or such as at least 20 MHC/peptide monomers. In another embodiment the MHC/peptide multimer comprises from 2 to 50 MHC/peptide monomers, such as from 2 to 4 MHC/peptide monomers, such as from 4 to 6 MHC/peptide monomers, such as from 6 to 8 MHC/peptide monomers, such as from 8 to 10 MHC/peptide monomers, such as from 10 to 12 MHC/peptide monomers, such as from 12 to 14 MHC/peptide monomers, such as from 14 to 16 MHC/peptide monomers, such as from 16 to 18 MHC/peptide monomers, such as from 18 to 20 MHC/peptide monomers, such as from 20 to 25 MHC/peptide monomers, such as from 25 to 30 MHC/peptide monomers, such as from 30 to 40 MHC/peptide monomers, such as from 40 to 50 MHC/peptide monomers, or any combination of these intervals. In one aspect the MHC/peptide multimer comprises no more than 30 MHC/peptide monomers in total, such as no more than 25 MHC/peptide monomers, such as no more than 20 MHC/peptide monomers, such as no more than 15 MHC/peptide monomers, or no more than 10 MHC/peptide monomers in total.

In a specific embodiment, the MHC/peptide multimer comprises at least 2, 3, 4, 5, 6, 7, 8, 9, 10, 11, 12, 13, 14, 15, 16, 17, 18, 19, 20, 21, 22, 23, 24 or 25 MHC/peptide monomers or has 2, 3, 4, 5, 6, 7, 8, 9, 10, 11, 12, 13, 14, 15, 16, 17, 18, 19, 20, 21, 22, 23, 24 or 25 MHC/peptide monomers in total. The MHC/peptide multimer can comprise identical MHC/peptide monomers or all MHC/peptide monomers of the MHC/peptide multimer can be identical. In another embodiment the MHC/peptide multimer comprises different MHC/peptide monomers or all MHC/peptide monomers of the MHC/peptide multimer are different. The MHC/peptide multimer can comprise MHC Class I monomers or all MHC/peptide monomers of the MHC/peptide multimer can be MHC Class I monomers. Alternatively, the MHC/peptide multimer can comprise MHC Class II monomers or all MHC/peptide monomers of the MHC/peptide multimer can be MHC Class II monomers. In another embodiment the MHC/peptide multimer comprises MHC Class I and MHC Class II monomers or all MHC/peptide monomers of the MHC/peptide multimer are either MHC Class I monomers or MHC Class II monomers. In one embodiment some of the MHC/peptide monomers or all of the MHC/peptide monomers on a MHC/peptide multimer have identical peptides. In another embodiment some of the MHC/peptide monomers or all of the MHC/peptide monomers on a MHC/peptide multimer have different peptides. The MHC/peptide multimer can comprise at least 2, such as at least 3, 4, 5, 6, 7, 8, 9, 10, 11, 12, 13, 14, 15, 16, 17, 18, 19 or 20 MHC/peptide monomers which comprise different peptides.

The MHC/peptide multimer may comprise one or more labels such as at least two labels. These labels can all be different or identical or some the labels can be identical and some different. In one embodiment the labels comprise at least one fluorescent label and/or at least one oligonucleotide label. In a specific embodiment the at least one oligonucleotide on a MHC/peptide multimer comprises one or more of: barcode region, 5′ first primer region (forward), 3′ second primer region (reverse), random nucleotide region, connector molecule, stability-increasing components, short nucleotide linkers in between any of the above-mentioned components, adaptors for sequencing and annealing region. MHC/peptide multimers are described in detail in WO02072631, WO2008116468, WO2009003492 and WO2020127222, which hereby are incorporated by reference.

The present disclosure relates to peptide-major histocompatibility complex (MHC)/peptide multimers comprising at least two MHC/peptide monomers, wherein at least one MHC/peptide monomer comprises a peptide derived from SARS-CoV-2 B.1.1.7, SARS-CoV-2 B1.351. SARS-CoV-2 P.1. and/or SARS-CoV-2 CAL.20C. In a preferred embodiment the MHC/peptide multimer comprises at least two MHC/peptide monomers, wherein at least one MHC/peptide monomer comprises a peptide which comprises, consists of, or consists essentially of an amino acid sequence selected from the sequences set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 Spike (S) protein such as a SARS-CoV-2 Spike (S) protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 Membrane (M) protein such as a SARS-CoV-2 Membrane (M) protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 Nucleocapsid (N) protein such as a SARS-CoV-2 Nucleocapsid (N) protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 Envelope (E) protein such as a SARS-CoV-2 Envelope (E) protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 ORF3a protein such as a SARS-CoV-2 ORF3a protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 ORF7a protein such as a SARS-CoV-2 ORF7a protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 ORF8 protein such as a SARS-CoV-2 ORF8 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp1 protein such as a SARS-CoV-2 nsp1 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp2 protein such as a SARS-CoV-2 nsp2 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp3 protein such as a SARS-CoV-2 nsp3 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp6 protein such as a SARS-CoV-2 nsp6 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp9 protein such as a SARS-CoV-2 nsp9 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp10 protein such as a SARS-CoV-2 nsp10 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp12 protein such as a SARS-CoV-2 nsp12 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp13 protein such as a SARS-CoV-2 nsp13 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp14 protein such as a SARS-CoV-2 nsp14 protein derived peptide set forth in Table 2 (SEQ ID NOS: 1 to 292) and/or Table 3 (SEQ ID NOS: 293 to 347). The MHC/peptide multimer can comprise at least one MHC/peptide monomer comprising a peptide derived from SARS-CoV-2 nsp15 protein such as a SARS-CoV-2 nsp15 protein derived peptide set forth in Table 2 (SEQ ID NOS: 1 to 292) and/or Table 3 (SEQ ID NOS: 293 to 347). The MHC/peptide multimer can comprise more than one of the different MHC/peptide monomers listed above, e.g., comprise at least 2, 3, 4, 5, 6, 7, 8, 9, 10 or more than 10 different MHC/peptide monomers by combining any of the above embodiments.

In certain embodiments, the at least two MHC/peptide monomers can be identical and/or different. In one embodiment the MHC/peptide multimer comprises some identical and some different MHC/peptide monomers or alternatively all the MHC/peptide monomers can be different. In a specific embodiment the MHC/peptide multimer comprises at least one MHC/peptide monomer which comprises a peptide comprising, consisting of, or consisting essentially of an amino acid sequence selected from the SARS-CoV-2 B.1.1.7 derived sequences set forth in Table 2 (SEQ ID NOS: 1 to 292) and/or Table 3 (SEQ ID NOS: 293 to 347). In a further embodiment the MHC/peptide multimer comprises at least one MHC/peptide monomer which comprises a peptide comprising, consisting of, or consisting essentially of an amino acid sequence selected from the SARS-CoV-2 B.1.351 derived sequences set forth in Table 2 (SEQ ID NOS: 1 to 292) and/or Table 3 (SEQ ID NOS: 293 to 347). In another embodiment the MHC/peptide multimer comprises at least one MHC/peptide monomer which comprises a peptide comprising, consisting of, or consisting essentially of an amino acid sequence selected from the SARS-CoV-2 P.1. derived sequences set forth in Table 2 (SEQ ID NOS: 1 to 292) and/or Table 3 (SEQ ID NOS: 293 to 347).

In a further embodiment, the MHC/peptide multimer comprises at least one MHC/peptide monomer which comprises a peptide comprising, consisting of, or consisting essentially of an amino acid sequence selected from the SARS-CoV-2 CAL.20C derived sequences set forth in Table 2 (SEQ ID NOS: 1 to 292) and/or Table 3 (SEQ ID NOS: 293 to 347). The MHC/peptide multimer can comprise more than one of the different MHC/peptide monomers listed above, e.g., comprise at least 2, 3, 4, 5, 6, 7, 8, 9, 10 or more than 10 different MHC/peptide monomers by combining any of the above embodiments.

The MHC/peptide multimer can comprise at least 3, such as at least 4, 5, 6, 7, 8, 9, 10, 11, 12, 13, 14, 15, 16, 17, 18, 19 or 20 MHC/peptide monomers. The MHC/peptide multimer can in one embodiment comprise at least 3, such as at least 4, 5, 6, 7, 8, 9, 10, 11, 12, 13, 14, 15, 16, 17, 18, 19 or 20 identical MHC/peptide monomers. The MHC/peptide multimer can in another embodiment comprise at least 3, such as at least 4, 5, 6, 7, 8, 9, 10, 11, 12, 13, 14, 15, 16, 17, 18, 19 or 20 different MHC/peptide monomers. In a particular embodiment the MHC/peptide multimer comprises at least 3, such as at least 4, 5, 6, 7, 8, 9, 10, 11, 12, 13, 14, 15, 16, 17, 18, 19 or 20 of the MHC/peptide monomers which comprises a peptide which comprises, consists of, or consists essentially of an amino acid sequence selected from the sequences set forth in Table 2 (SEQ ID NOS: 1 to 292) and/or Table 3 (SEQ ID NOS: 293 to 347).

In a specific embodiment, the MHC/peptide multimer comprises at least 3, such as at least 4, 5, 6, 7, 8, 9, 10, 11, 12, 13, 14, 15, 16, 17, 18, 19 or 20 MHC/peptide monomers which comprises a peptide which comprises, consists of, or consists essentially of an amino acid sequence selected from the SARS-CoV-2 B.1.1.7 derived sequences set forth in Table 2 (SEQ ID NOS: 1 to 292) and/or Table 3 (SEQ ID NOS: 293 to 347). In another embodiment the MHC/peptide multimer comprises at least 3, such as at least 4, 5, 6, 7, 8, 9, 10, 11, 12, 13, 14, 15, 16, 17, 18, 19 or 20 MHC/peptide monomers which comprises a peptide which comprises, consists of, or consists essentially of an amino acid sequence selected from the SARS-CoV-2 B.1.351 derived sequences set forth in Table 2 (SEQ ID NOS: 1 to 292) and/or Table 3 (SEQ ID NOS: 293 to 347). In a further embodiment the MHC/peptide multimer comprises at least 3, such as at least 4, 5, 6, 7, 8, 9, 10, 11, 12, 13, 14, 15, 16, 17, 18, 19 or 20 MHC/peptide monomers which comprises a peptide which comprises, consists of, or consists essentially of an amino acid sequence selected from the SARS-CoV-2 P.1. derived sequences set forth in Table 2 (SEQ ID NOS: 1 to 292) and/or Table 3 (SEQ ID NOS: 293 to 347). In another embodiment the MHC/peptide multimer comprises at least 3, such as at least 4, 5, 6, 7, 8, 9, 10, 11, 12, 13, 14, 15, 16, 17, 18, 19 or 20 MHC/peptide monomers which comprises a peptide which comprises, consists of, or consists essentially of an amino acid sequence selected from the SARS-CoV-2 CAL.20C derived sequences set forth in Table 2 (SEQ ID NOS: 1 to 292) and/or Table 3 (SEQ ID NOS: 293 to 347). In another embodiment the MHC/peptide multimer comprises at least 3, such as at least 4, 5, 6, 7, 8, 9, 10, 11, 12, 13, 14, 15, 16, 17, 18, 19 or 20 MHC/peptide monomers which comprises a peptide which comprises, consists of, or consists essentially of an amino acid sequence selected from the SARS-CoV-2 Spike (S) protein, Membrane (M) protein, Nucleocapsid (N) protein, Envelope (E) protein, ORF3a, ORF7a, ORF8, nsp1, nsp2, nsp3, nsp6, nsp9, nsp10, nsp12, nsp13, nsp14 and/or nsp15 derived sequences set forth in Table 2 (SEQ ID NOS: 1 to 292) and/or Table 3 (SEQ ID NOS: 293 to 347). The MHC/peptide multimer can comprise more than one of the different MHC/peptide monomers listed above, e.g., comprise at least 2, 3, 4, 5, 6, 7, 8, 9, 10 or more than 10 different MHC/peptide monomers by combining any of the above embodiments.

This disclosure further relates to a composition comprising at least two MHC/peptide multimers as described above, such as at least 3, 4, 5, 6, 7, 8, 9, 10, 20, 30, 40, 50, 100 or 1000 MHC/peptide multimers. The MHC/peptide multimers in the composition can all be identical or different. Alternatively, some MHC/peptide multimers in the composition are identical and some are different. The composition can comprise different MHC/peptide multimers, such as at least 2, 3, 4, 5, 6, 7, 8, 9, 10, 15, 20, 25, 50, 100, 500 or 1000 different MHC/peptide multimers. The composition can in one embodiment comprise at least 2, 3, 4, 5, 6, 7, 8, 9, 10, 20, 30, 40, 50, 100 or 1000 different MHC/peptide multimers each comprising one or more peptides selected from one or more such as 2, 3, 4, 5, 6, 7, 8, 9 or 10 of the following groups:
one or more peptides derived from SARS-CoV-2 B.1.1.7, such as one or more SARS-CoV-2 B.1.1.7 derived peptides set forth in Table 2 (SEQ ID NOS: 1 to 292) and/or Table 3 (SEQ ID NOS: 293 to 347),
one or more peptides derived from SARS-CoV-2 B1.351. such as one or more SARS-CoV-2 B1.351 derived peptides set forth in Table 2 (SEQ ID NOS: 1 to 292) and/or Table 3 (SEQ ID NOS: 293 to 347)
one or more peptides derived from SARS-CoV-2 P.1, such as one or more SARS-CoV-2 P.1 derived peptides set forth in Table 2 (SEQ ID NOS: 1 to 292) and/or Table 3 (SEQ ID NOS: 293 to 347),
one or more peptides derived from SARS-CoV-2 CAL.20C, such as one or more SARS-CoV-2 CAL.20C derived peptides set forth in Table 2 (SEQ ID NOS: 1 to 292) and/or Table 3 (SEQ ID NOS: 293 to 347),
one or more peptides set forth in Table 2 (SEQ ID NOS: 1 to 292) and/or Table 3 (SEQ ID NOS: 293 to 347),
one or more peptides derived from the SARS-CoV-2 Spike (S) protein such as one or more SARS-CoV-2 Spike (S) protein derived peptides set forth in Table 2 (SEQ ID NOS: 1 to 292) and/or Table 3 (SEQ ID NOS: 293 to 347),
one or more peptides derived from the SARS-CoV-2 Membrane (M) protein such as one or more SARS-CoV-2 Membrane (M) protein derived peptides set forth in Table 2 (SEQ ID NOS: 1 to 292) and/or Table 3 (SEQ ID NOS: 293 to 347),
one or more peptides derived from the SARS-CoV-2 Nucleocapsid (N) protein such as one or more SARS-CoV-2 Nucleocapsid (N) protein derived peptides set forth in Table 2 (SEQ ID NOS: 1 to 292) and/or Table 3 (SEQ ID NOS: 293 to 347),
one or more peptides derived from the SARS-CoV-2 Envelope (E) protein such as one or more SARS-CoV-2 Envelope (E) protein derived peptides set forth in Table 2 (SEQ ID NOS: 1 to 292) and/or Table 3 (SEQ ID NOS: 293 to 347),
one or more peptides derived from the SARS-CoV-2 ORF3a protein such as one or more SARS-CoV-2 ORF3a protein derived peptides set forth in Table 2 (SEQ ID NOS: 1 to 292) and/or Table 3 (SEQ ID NOS: 293 to 347),
one or more peptides derived from the SARS-CoV-2 ORF7a protein such as one or more SARS-CoV-2 ORF7a protein derived peptides set forth in Table 2 (SEQ ID NOS: 1 to 292) and/or Table 3 (SEQ ID NOS: 293 to 347),
one or more peptides derived from the SARS-CoV-2 ORF8 protein such as one or more SARS-CoV-2 ORF8 protein derived peptides set forth in Table 2 (SEQ ID NOS: 1 to 292) and/or Table 3 (SEQ ID NOS: 293 to 347),
one or more peptides derived from the SARS-CoV-2 nsp1 protein such as one or more SARS-CoV-2 nsp1 protein derived peptides set forth in Table 2 (SEQ ID NOS: 1 to 292) and/or Table 3 (SEQ ID NOS: 293 to 347),
one or more peptides derived from the SARS-CoV-2 nsp2 protein such as one or more SARS-CoV-2 nsp2 protein derived peptides set forth in Table 2 (SEQ ID NOS: 1 to 292) and/or Table 3 (SEQ ID NOS: 293 to 347),
one or more peptides derived from the SARS-CoV-2 nsp3 protein such as one or more SARS-CoV-2 nsp3 protein derived peptides set forth in Table 2 (SEQ ID NOS: 1 to 292) and/or Table 3 (SEQ ID NOS: 293 to 347),
one or more peptides derived from the SARS-CoV-2 nsp6 protein such as one or more SARS-CoV-2 nsp6 protein derived peptides set forth in Table 2 (SEQ ID NOS: 1 to 292) and/or Table 3 (SEQ ID NOS: 293 to 347),
one or more peptides derived from the SARS-CoV-2 nsp9 protein such as one or more SARS-CoV-2 nsp9 protein derived peptides set forth in Table 2 (SEQ ID NOS: 1 to 292) and/or Table 3 (SEQ ID NOS: 293 to 347),
one or more peptides derived from the SARS-CoV-2 nsp10 protein such as one or more SARS-CoV-2 nsp10 protein derived peptide set forth in Table 2 (SEQ ID NOS: 1 to 292) and/or Table 3 (SEQ ID NOS: 293 to 347),
one or more peptides derived from the SARS-CoV-2 nsp12 protein such as one or more SARS-CoV-2 nsp12 protein derived peptides set forth in Table 2 (SEQ ID NOS: 1 to 292) and/or Table 3 (SEQ ID NOS: 293 to 347),
one or more peptides derived from the SARS-CoV-2 nsp13 protein such as one or more SARS-CoV-2 nsp13 protein derived peptides set forth in Table 2 (SEQ ID NOS: 1 to 292) and/or Table 3 (SEQ ID NOS: 293 to 347),
one or more peptides derived from the SARS-CoV-2 nsp14 protein such as one or more SARS-CoV-2 nsp14 protein derived peptides set forth in Table 2 (SEQ ID NOS: 1 to 292) and/or Table 3 (SEQ ID NOS: 293 to 347) and
one or more peptides derived from the SARS-CoV-2 nsp15 protein such as one or more SARS-CoV-2 nsp15 protein derived peptides set forth in Table 2 (SEQ ID NOS: 1 to 292) and/or Table 3 (SEQ ID NOS: 293 to 347).

In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from SARS-CoV-2 B.1.1.7, such as one or more SARS-CoV-2 B.1.1.7 derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from SARS-CoV-2 B1.351. such as one or more SARS-CoV-2 B1.351 derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from SARS-CoV-2 P.1, such as one or more SARS-CoV-2 P.1 derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from SARS-CoV-2 CAL.20C, such as one or more SARS-CoV-2 CAL.20C derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 Spike (S) protein such as one or more SARS-CoV-2 Spike (S) protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 Membrane (M) protein such as one or more SARS-CoV-2 Membrane (M) protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 Nucleocapsid (N) protein such as one or more SARS-CoV-2 Nucleocapsid (N) protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 Envelope (E) protein such as one or more SARS-CoV-2 Envelope (E) protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 ORF3a protein such as one or more SARS-CoV-2 ORF3a protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 ORF7a protein such as one or more SARS-CoV-2 ORF7a protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 ORF8 protein such as one or more SARS-CoV-2 ORF8 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp1 protein such as one or more SARS-CoV-2 nsp1 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp2 protein such as one or more SARS-CoV-2 nsp2 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp3 protein such as one or more SARS-CoV-2 nsp3 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp6 protein such as one or more SARS-CoV-2 nsp6 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp9 protein such as one or more SARS-CoV-2 nsp9 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp10 protein such as one or more SARS-CoV-2 nsp10 protein derived peptide set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp12 protein such as one or more SARS-CoV-2 nsp12 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp13 protein such as one or more SARS-CoV-2 nsp13 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp14 protein such as one or more SARS-CoV-2 nsp14 protein derived peptides set forth in Table 2 (SEQ ID NOS: 1 to 292) and/or Table 3 (SEQ ID NOS: 293 to 347). In one embodiment the composition comprises at least 1 such as at least 2, 3, 4, 5, 6, 7, 8, 9, 10, 20, 30, 40, 50, 100 or 1000 identical or different MHC/peptide multimers each comprising one or more peptides derived from the SARS-CoV-2 nsp15 protein such as one or more SARS-CoV-2 nsp15 protein derived peptides set forth in Table 2 (SEQ ID NOS: 1 to 292) and/or Table 3 (SEQ ID NOS: 293 to 347). Any of the above composition embodiments can be combined in any order.

In humans there are three major different genetic loci that encode MHC class II molecules: HLA-DR, HLA-DP, and HLA-DQ, each formed of two polypeptides, alpha and beta chains (A and B genes). For example, HLA-DQA1*01, HLA-DRB1*01, and HLA-DRB1*03 are different MHC class II alleles that can be expressed from these loci. It should be further noted that non-classical human MHC class II molecules such as HLA-DM and HL-DOA (homolog in mice is H2-DM and H2-0) are also encompassed by the present disclosure. In some embodiments, the MHC/peptide multimer is an HLA/peptide multimer selected from the group consisting of HLA-DP/peptide multimer, HLA-DQ/peptide multimer, HLA-DR/peptide multimer, HLA-DM/peptide multimer and HLA-DO/peptide multimer.

An MHC/peptide multimer may be a multimer where the heavy chain of the MHC is biotinylated, which allows combination as a tetramer with streptavidin. MHC-peptide tetramers have increased avidity for the appropriate T cell receptor (TCR) on T lymphocytes. The multimers can also be attached to paramagnetic particles or magnetic beads to facilitate removal of non-specifically bound reporter and cell sorting. Multimer staining does not kill the labelled cells, thus, cell integrity is maintained for further analysis. In some embodiments, the MHC/peptide multimer of the present disclosure is particularly suitable for isolating and/or identifying a population of CD8+ T cells having specificity for the peptide of the present disclosure (in a flow cytometry assay).

The peptides or MHC class I or class II multimer as described herein is particularly suitable for detecting T cells specific for one or more peptides of the present disclosure. The peptide(s) and/or the MHC/multimer complex of the present disclosure is particularly suitable for diagnosing coronavirus infection in a subject. For example, the method comprises obtaining a blood or PBMC sample obtained from the subject with an amount of a least peptide of the present disclosure and detecting at least one T cell displaying a specificity for the peptide. Another diagnostic method of the present disclosure involves the use of a peptide of the present disclosure that is loaded on multimers as described above, so that the isolated CD8+ or CD4+ T cells from the subject are brought into contact with the multimers, at which the binding, activation and/or expansion of the T cells is measured. For example, following the binding to antigen presenting cells, e.g., those having the MHC class I or class II multimer, the number of CD8+ and/or CD4+ cells binding specifically to the HLA-peptide multimer may be quantified by measuring the secretion of lymphokines/cytokines, division of the T cells, or standard flow cytometry methods, such as, for example, using fluorescence activated cell sorting (FACS). The multimers can also be attached to paramagnetic ferrous or magnetic beads to facilitate removal of non-specifically bound reporter and cell sorting.

The MHC class I or class II peptide multimers as described herein can also be used as therapeutic agents. The peptide and/or the MHC class I or class II peptide multimers of the present disclosure are suitable for treating or preventing a coronavirus infection in a subject. The MHC Class I or Class II multimers can be administered in soluble form or loaded on nanoparticles.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein or peptide, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

As used herein, the term “antigen” and the term “epitope” refers to a molecule or substance capable of stimulating an immune response. In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present disclosure includes, in one embodiment, pre-processed peptides that are attached to the anti-CD40 antibody (or fragment thereof) that directs the peptides against which an enhanced immune response is sought directly to antigen presenting cells.

The present disclosure includes methods for specifically identifying the epitopes within antigens most likely to lead to the immune response sought for the specific sources of antigen presenting cells and responder T cells.

As used herein, the term “T cell epitope” refers to a specific amino acid that when present in the context of a Major or Minor Histocompatibility Complex provides a reactive site for a T cell receptor. The T-cell epitopes or peptides that stimulate the cellular arm of a subject's immune system are short peptides of about 8-25 amino acids. T-cell epitopes are recognized by T cells from animals that are immune to the antigen of interest. These T-cell epitopes or peptides can be used in assays such as the stimulation of cytokine release or secretion or evaluated by constructing major histocompatibility (MHC) proteins containing or “presenting” the peptide. Such immunogenically active fragments are often identified based on their ability to stimulate lymphocyte proliferation in response to stimulation by various fragments from the antigen of interest.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTLs”). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of effector and/or suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” and “vaccine” refer to a composition that comprises an antigenic molecule where administration of the composition to a subject or patient results in the development in the subject of a humoral and/or a cellular immune response to the antigenic molecule of interest. “Vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen) or therapeutic (e.g., reducing symptoms or aberrant conditions associated with infection). The administration of vaccines is referred to vaccination.

In some examples, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic bacterium or virus).

The present disclosure provides nucleic acid molecules, specifically polynucleotides, primary constructs and/or mRNA that encode one or more polynucleotides that express one or more peptides or proteins, comprising, consisting of, or consisting essentially of an amino acid sequence selected from those sequences set forth in Table 2 or Table 3, or a subsequence, portion, homologue, variant or derivative thereof for use in immune modulation. The term “nucleic acid” refers to any compound and/or substance that comprise a polymer of nucleotides, referred to herein as polynucleotides. Exemplary nucleic acids or polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), including diastereomers of LNAs, functionalized LNAs, or hybrids thereof.

One method of immune modulation of the present disclosure includes direct or indirect gene transfer, i.e., local application of a preparation containing the one or more polynucleotides (DNA, RNA, mRNA, etc.) that expresses the one or more peptides or proteins, comprising, consisting of, or consisting essentially of an amino acid sequence selected from those sequences set forth in Table 2 or Table 3, or a subsequence, portion, homologue, variant or derivative thereof. A variety of well-known vectors can be used to deliver to cells the one or more polynucleotides or the peptides or proteins expressed by the polynucleotides, including but not limited to adenoviral vectors and adeno-associated vectors. In addition, naked DNA, liposome delivery methods, or other novel vectors developed to deliver the polynucleotides to cells can also be beneficial. Any of a variety of promoters can be used to drive peptide or protein expression, including but not limited to endogenous promoters, constitutive promoters (e.g., cytomegalovirus, adenovirus, or SV40), inducible promoters (e.g., a cytokine promoter such as the interleukin-1, tumor necrosis factor-alpha, or interleukin-6 promoter), and tissue specific promoters to express the immunogenic peptides or proteins of the present disclosure.

The immunization may include adenovirus, adeno-associated virus, herpes virus, vaccinia virus, retroviruses, or other viral vectors with the appropriate tropism for cells likely to present the antigenic peptide(s) or protein(s) may be used as a gene transfer delivery system for a therapeutic peptide(s) or protein(s), comprising, consisting of, or consisting essentially of an amino acid sequence selected from those sequences set forth in Table 2 or Table 3, or a subsequence, portion, homologue, variant or derivative thereof, gene expression construct. Viral vectors which do not require that the target cell be actively dividing, such as adenoviral and adeno-associated vectors, are particularly useful when the cells are accumulating, but not proliferative. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; and Miller and Rosman, Bio Techniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

The immunization may also include inserting the one or more polynucleotides (DNA, RNA, mRNA, etc.) that express the one or more peptides or proteins, comprising, consisting of, or consisting essentially of an amino acid sequence selected from those sequences set forth in Table 2 or Table 3, or a subsequence, portion, homologue, variant or derivative thereof into the viral vector, along with another gene which encodes the ligand for a receptor on a specific target cell, for example, such that the vector is now target specific. Viral vectors can be made target specific by attaching, for example, a sugar, a glycolipid, or a protein. Targeting can also be accomplished by using an antibody to target the viral vector. Those of skill in the art will know of, or can readily ascertain without undue experimentation, specific polynucleotide sequences which can be inserted into the viral genome or attached to a viral envelope to allow target specific delivery of the viral vector containing the gene.

Since recombinant viruses are defective, they require assistance in order to produce infectious vector particles. This assistance can be provided, for example, by using helper cell lines that contain plasmids encoding all of the structural genes of the virus under the control of regulatory sequences within the viral genome. These plasmids are missing a nucleotide sequence which enables the packaging mechanism to recognize a polynucleotide transcript for encapsidation. These cell lines produce empty virions, since no genome is packaged. If a viral vector is introduced into such cells in which the packaging signal is intact, but the structural genes are replaced by other genes of interest, the vector can be packaged and vector virion produced.

Viral or non-viral approaches may also be employed for the introduction of one or more therapeutic polynucleotides that express the one or more peptides or proteins, comprising, consisting of, or consisting essentially of an amino acid sequence selected from those sequences set forth in Table 2 or Table 3, or a subsequence, portion, homologue, variant or derivative thereof, into polynucleotide-encoding polynucleotide into antigen presenting cells. The polynucleotides may be DNA, RNA, mRNA that directly encode the one or more peptides or proteins of the present disclosure, or may be introduced as part of an expression vector.

Another example of an immunization includes colloidal dispersion systems that include macromolecule complexes, nanocapsules, microspheres, beads, and lipid-based systems including oil-in-water emulsions, micelles, mixed micelles, and liposomes and the one or more polynucleotides that express the one or more peptides or proteins, comprising, consisting of, or consisting essentially of an amino acid sequence selected from those sequences set forth in Table 2 or Table 3, or a subsequence, portion, homologue, variant or derivative thereof. One non-limiting example of a colloidal system for use with the present disclosure is a liposome. Liposomes are artificial membrane vesicles which are useful as delivery vehicles in vitro and in vivo. It has been shown that large unilamellar vesicles (LUV), which range in size from 0.2-4.0 micrometers that can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (Zakut and Givol, supra) encapsulation of the genes of interest at high efficiency while not compromising their biological activity; (Fearnhead, et al., supra) preferential and substantial binding to a target cell in comparison to non-target cells; (Korsmeyer, S. J., supra) delivery of the aqueous contents of the vesicle to the target cell cytoplasm at high efficiency; and (Kinoshita, et al., supra) accurate and effective expression of genetic information (Mannino, et al., Bio Techniques, 6:682, 1988).

The composition for immunizing the subject or patient may, in certain embodiments comprise a combination of phospholipid, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization, specifically, cells that can become infected with a coronavirus or interact with the proteins, peptides, and/or gene products of a coronavirus, e.g., immune cells.

For any of the above approaches, the immune modulating polynucleotide construct, composition, or formulation is preferably applied to a site that will enhance the immune response. For example, the immunization may be intramuscular, intraperitoneal, enteral, parenteral, intranasal, intrapulmonary, or subcutaneous. In the gene delivery constructs of the instant disclosure, polynucleotide expression is directed from any suitable promoter (e.g., the human cytomegalovirus, simian virus 40, actin or adenovirus constitutive promoters; or the cytokine or metalloprotease promoters for activated synoviocyte specific expression).

In one example of the immune modifying peptide(s) or protein(s) include polynucleotides, constructs and/or mRNAs that express the one or more polynucleotides that express the one or more peptides or proteins, comprising, consisting of, or consisting essentially of an amino acid sequence selected from those sequences set forth in Table 2 or Table 3, or a subsequence, portion, homologue, variant or derivative thereof, that are designed to improve one or more of the stability and/or clearance in tissues, uptake and/or kinetics, cellular access by the peptide(s) or protein(s), translational, mRNA half-life, translation efficiency, immune evasion, protein production capacity, accessibility to circulation, peptide(s) or protein(s) half-life and/or presentation in the context of MHC on antigen presenting cells.

The present disclosure contemplates immunization for use in both active and passive immunization embodiments. Immunogenic compositions, proposed to be suitable for use as a vaccine, may be prepared most readily directly from immunogenic peptides, proteins, monomers, multimers and/or peptide-MHC complexes prepared in a manner disclosed herein. The antigenic material is generally processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The preparation of immunizations (also referred to as vaccines) that contain the immunogenic proteins of the present disclosure as active ingredients is generally well understood in the art, as exemplified by United States Letters Patents U.S. Pat. Nos. 4,608,251; 4,601,903; 4,599,231; 4,599,230; 4,596,792; and 4,578,770, all incorporated herein by reference. Typically, such immunizations are prepared as injectables. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines.

The immunization is/are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° to 101° C. for 30 second to 2-minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as Hudson and Cranage, Vaccine Protocols, 2003 Humana Press, relevant portions incorporated herein by reference.

Techniques and compositions for making useful dosage forms using the present disclosure are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins, 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif.)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif. (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portion incorporated herein by reference.

As used herein, the term “effective amount” or “effective dose” refers to that amount of the peptide or protein T cell epitopes of the disclosure sufficient to induce immunity, to prevent and/or ameliorate an infection or to reduce at least one symptom of an infection and/or to enhance the efficacy of another dose of peptide or protein T cell epitopes. An effective dose may refer to the amount of peptide or protein T cell epitopes sufficient to delay or minimize the onset of an infection. An effective dose may also refer to the amount of peptide or protein T cell epitopes that provides a therapeutic benefit in the treatment or management of an infection. Further, an effective dose is the amount with respect to peptide or protein T cell epitopes of the disclosure alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, an infectious disease, and more particularly, a coronavirus infection. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as peptide or protein T cell epitopes of the disclosure, or can be administered separately. Either the protein or an expression vector encoding the protein can be administered to produce an immunostimulatory effect.

As used herein, in certain embodiments, the term “protective immune response” or “protective response” refers to an immune response mediated by antibodies against an infectious agent, which is exhibited by a vertebrate (e.g., a human), which prevents or ameliorates an infection or reduces at least one symptom thereof. Peptide and protein T cell epitopes of the disclosure can stimulate the production of antibodies that, for example, neutralize infectious agents, blocks infectious agents from entering cells, blocks replication of said infectious agents, and/or protect host cells from infection and destruction. In other embodiments, the term can also refer to an immune response that is mediated by T-lymphocytes and/or other white blood cells against an infectious agent, exhibited by a vertebrate (e.g., a human), that prevents or ameliorates flavivirus infection or reduces at least one symptom thereof. Peptide and protein T cell epitopes of the disclosure can stimulate the T cell responses that, for example, neutralize infectious agents, kill virus infected cells, blocks infectious agents from entering cells, blocks replication of said infectious agents, and/or protect host cells from infection and destruction.

The terms “biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The terms “virus” or “virus particle” are used according to its plain ordinary meaning within Virology and refers to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins. In embodiments, the virus is a coronavirus. Non-limiting examples of coronaviruses (CoV) from which T cell epitopes can be identified include, e.g., SARS-CoV (SARS-CoV-1), MERS-CoV, and SARS-CoV-2, but also betacoronaviruses, e.g., HCoV-OC43, HCoVHKU1, HCoV-229E and alphacoronaviruses such as HCoV-NL63, and/or other coronaviruses endemic in humans. The viral genome of coronaviruses encodes at least the following structure proteins, the spike (S), envelope (E), membrane (M), and nucleocapsid (N) proteins. The S glycoprotein is responsible for binding the host receptor via the receptor-binding domain (RBD) in its S1 subunit, as well as the subsequent membrane fusion and viral entry driven by its S2 subunit. Gene sequencing of SARS-CoV-2 showed that this novel coronavirus, a betacoronavirus, is related to the MERS-CoV and the SARS-CoV. SARS-CoV, MERS-CoV, and SARS-CoV-2 belong to the betacoronavirus genus and are highly pathogenic zoonotic viruses. Thus, the present disclosure can be used not only to determine antigenic peptides from the three highly pathogenic betacoronaviruses, but also low-pathogenicity betacoronaviruses, such as, HCoV-OC43, HCoVHKU1, HCoV-NL63 and HCoV-229E, are also endemic in humans. In certain specific embodiments, the coronavirus is SARS-CoV-2, including novel mutants of SARS-CoV-2 that include mutants from five clades (19A, 19B, 20A, 20B, and 20C) according to Nextstrain, in GISAID nomenclature which divides them into seven clades (L, O, V, S, G, GH, and GR), and/or PANGOLIN nomenclature which divides them into six major lineages (A, B, B.1, B.1.1, B.1.177, B.1.1.7). Notable mutations of SARS-CoV-2 include, e.g., D614G, P681H, N501Y, 69-70del, P681H, Y453F, 69-70deltaHV, N501Y, K417N, E484K, N501Y, and E484K.

As used herein, a “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “contacting” is used in accordance with its plain ordinary meaning and refers to the process of allowing at least two distinct species to become sufficiently proximal to react, interact or physically touch. It should be appreciated; however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be, for example, an amino acid sequence, protein, or peptide as provided herein and an immune cell, such as a T cell.

As used herein, a “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The terms “subject” or “subject in need thereof” refers to a living organism who is at risk of or prone to having a disease or condition, or who is suffering from a disease or condition that can be treated by administration of a composition or pharmaceutical composition as provided herein. Non-limiting examples include humans and other primates, but also includes non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

The terms “disease” or “condition” refer to a state of being or health status of a patient or subject capable of being treated with a compound, pharmaceutical composition, or method provided herein. In embodiments, a patient or subject is human. In embodiments, the disease is coronavirus infection. In certain alternative embodiments, the disease is SARS-CoV-2 infection. In still other embodiments, the disease is COVID-19.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated or the disorder resulting from viral infection. Also, a therapeutic benefit is achieved with the eradication or amelioration of one or more of the physiological symptoms associated with viral infection or the underlying disorder such that an improvement is observed in the patient, notwithstanding that the patient may still be afflicted with the underlying disorder or may still be infected. For prophylactic benefit, the compositions may be administered to a patient at risk of viral infection, of developing a particular disease, or to a patient reporting one or more of the physiological symptoms of a disease, even though a diagnosis of this disease may not have been made. Treatment includes preventing the infection or disease, that is, causing the clinical symptoms of the disease not to develop by administration of a protective composition prior to infection or the induction of the disease; suppressing the disease, that is, causing the clinical symptoms of the disease or infection not to develop by administration of a protective composition after the inductive event or infection but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Treatment” can also refer to any of (i) the prevention of infection or reinfection, as in a traditional vaccine, (ii) the reduction or elimination of symptoms, and (iii) the substantial or complete elimination of the pathogen in question. Treatment may be affected prophylactically (prior to infection) or therapeutically (following infection).

In addition, in certain embodiments, “treatment,” “treat,” or “treating” refers to a method of reducing the effects of one or more symptoms of infection with a coronavirus. Thus, in the disclosed method, treatment can refer to a 10%, 20%, 30%, 40%, 50%, 60%, 70%, 80%, 90%, or 100% reduction in the severity of an established infection, disease, condition, or symptom of the infection, disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition and/or complete prevention of infection. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to recognition of an infection, disease or condition by signs and symptoms. Diagnosing can refer to determination of whether a subject has an infection or disease. Diagnosis may refer to determination of the type of disease or condition a subject has or the type of virus the subject is infected with.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, β-galactosidase, β-glucoronidase or β-lactamase. Such enzymes may be used in combination with a chromogen, a fluorogenic compound or a luminogenic compound to generate a detectable signal.

The peptide(s) or protein(s) of the present disclosure can also be used in binding assays including, but are not limited to, immunoassays such as competitive and non-competitive assay systems using techniques such as western blots, radioimmunoassays, ELISA (enzyme linked immunosorbent assay), “sandwich” immunoassays, Meso Scale Discovery (MSD, Gaithersburg, Md.), immunoprecipitation assays, ELISPOT, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York, relevant portions incorporated herein by reference).

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the disclosure can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the compositions provided herein including embodiments thereof, augments the composition's immune response. Generally, adjuvants are non-toxic, have high-purity, are degradable, and are stable.

Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the disclosure are saponin adjuvants, such as Stimulon™ (QS 21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 α and β peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1a and p and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this disclosure, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the disclosure. One of skill in the art will recognize that other pharmaceutical excipients are useful in the present disclosure.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present disclosure may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present disclosure can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present disclosure can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present disclosure can also be delivered as nanoparticles.

The present disclosure describes methods utilizing and compositions comprising or expressing T cell epitopes, T cell epitope-containing peptides, and T cell epitope-containing proteins associated with binding to a subset of the naturally occurring MHC Class II and/or MHC Class I molecules within the human population. Compositions comprising or expressing one or more of the disclosed peptides (e.g., the amino acid sequences set forth in Table 2 (SEQ ID NOS: 1 to 292) or Table 3 (SEQ ID NOS: 293 to 347) or polynucleotides encoding the same, covering different HLA Class II and/or MHC Class I alleles, capable of generating a treatment acting broadly on a population level are disclosed herein. As uses throughout the specification when referring to the use peptide epitopes, the composition can comprise or express 1, 2, 3, 4, 5, 6, 7, 8, 9, 10, 11, 12, 13, 14, 15, 16, 17, 18, 19, 20, 20, 25, 30, 40, 50, 60, 70, 75, 89, 90, 100, 110, 120, 125, 130, 140, 150, 160, 179, 175, 180, 190, 200, 225, 250, 275, 300, 325, or 347 peptides. As the antigen repertoire of MHC Class I and MHC Class II alleles varies from one individual to another and from one ethnic population to another, it is challenging to provide vaccines or peptide or epitopes-based immunotherapies that can be offered to subjects of any geographic region in the world or provide sufficient protection against infection across a wide segment of the populations unless numerous epitopes or peptides are included (e.g., in a vaccine). Taking into consideration the need for a single vaccine formulation that can provide protection across populations, if it desirable to provide a treatment containing or expressing proteins, peptides or epitopes that will provide protection against infection amongst the majority of the worldwide population. Also, taking into consideration the enormous costs and risks in the clinical development of new treatments and the increasing demands from regulatory bodies to meet high standards for toxicity testing, dose justification, safety and efficacy trials, it is desirable to provide treatments containing or expressing as few peptides as possible, but at the same time to be able to treat the majority of subjects in a worldwide population with a single immunotherapy. Such a product should comprise as a first requirement an expression or inclusion of combination of epitopes or peptides that are able to bind the worldwide MHC Class I and/or MHC Class II allele repertoire, and the resulting peptide-MHC complexes should as a second requirement be recognized by the T cells of the subject so as to induce the desired immunological reactions.

The present disclosure further provides the following methods:
A method for monitoring an immune response relevant to a coronavirus infection comprising one or more steps of:
   i) providing one or more MHC/peptide multimers or a composition comprising at least one MHC/peptide multimer according to the disclosure,
   ii) providing a sample comprising a population of T cells, and
   iii) measuring the presence, frequency, number, activity and/or state of T cells specific for said one or more MHC/peptide multimers,
thereby monitoring said immune response relevant to a coronavirus infection.
A method for diagnosing a coronavirus infection comprising one or more steps of:
   i) providing one or more MHC/peptide multimers or a composition comprising at least one MHC/peptide multimer according to the disclosure,
   ii) providing a sample comprising a population of T cells, and
   iii) measuring the presence, frequency, number, activity and/or state of T cells specific for said one or more MHC/peptide multimers, thereby diagnosing said coronavirus infection.
A method for isolation of one or more antigen-specific T cells, said method comprising one or more steps of
   i) providing a sample comprising a population of T cells,
   ii) providing one or more MHC/peptide multimers or a composition comprising at least one MHC/peptide multimer according to the disclosure,
   iii) contacting said MHC/peptide multimers or composition with said sample comprising a population of T cells, and
   iv) isolating T cells specific for said MHC/peptide multimers or composition.
A method for detecting an antigen-specific T cell response comprising one or more steps of:
   i) providing a sample comprising a population of T cells,
   ii) providing one or more MHC/peptide multimers or a composition comprising at least one MHC/peptide multimer according to the disclosure,
   iii) contacting said MHC/peptide multimers or composition with said sample, and
   iv) measuring the presence, frequency, number, activity and/or state of T cells specific for said MHC/peptide multimers or composition, thereby detecting said antigen-specific T cell response.

The present disclosure provides improved epitope or peptide combinations for modulating an immune response, for treating a subject for an infection or aberrant immune response, and for use in diagnostic methods and kits comprising such peptide combinations. It is another object of the disclosure to provide epitope or peptide combinations exhibiting very good HLA Class I and Class II coverage in a worldwide population and being immunologically potent in a worldwide population. It is another object of the disclosure to provide epitope or peptide combinations having good cross reactivity to other viral strains, including co-circulating strains (for example, mutants) of coronaviruses, including SARS-CoV-2, common cold coronaviruses, as well as SARS-CoV, MERS, etc. It is another object of the disclosure to provide epitope or peptide combinations of a relatively small number of epitopes or peptides yet obtaining at least 70%, and more preferably around 90-100% donor coverage in a donor cohort representative of a worldwide population. In certain embodiments, this is achieved by selecting one or more immunodominant and/or immunoprevalent proteins (e.g., a SARS-CoV-2 protein) or subsequences, portions, homologues, variants or derivatives thereof for use in the methods and compositions of the present disclosure, wherein said immunodominant and/or immunoprevalent proteins or subsequences, portions, homologues, variants or derivatives thereof comprise two or more epitopes that are immunodominant and/or immunoprevalant. In some embodiments, the two or more epitopes comprise two to ten epitopes and/or polynucleotides encoding the same. Another object of the disclosure is to provide epitope combinations which are so immunologically potent that even at very low doses of epitopes, the percentage of responding donors can be retained at a very high level in a donor cohort representative of a worldwide population. Another object of the disclosure is to provide epitope combinations which have minor risk of inducing IgE-mediated adverse events. An additional object of the disclosure is to provide proteins, peptides, or nucleic acids containing or expressing epitopes or combinations of such proteins, peptides or nucleic acids which have a sufficient solubility profile for being formulated in a pharmaceutical product, preferably which have acceptable estimated in vivo stability. One further objective of the disclosure is to select epitopes for use in the compositions and methods described herein, based on one or both of their immunodominance or immunoprevalence. A still further object of the disclosure is to select such epitopes and epitopes combinations not only in accordance with those embodiments previously described, but also those epitopes and epitope combinations capable of eliciting a B cell response and T cell response (e.g., selecting one or more peptides for use in the methods and compositions described herein capable of generating a T cell and antibody response in a subject).

Provided herein are methods and compositions for diagnosing, treating, and immunizing against a coronavirus, including methods and compositions of detecting an immune response or immune cells relevant to a coronavirus infection. These methods and compositions include vaccines, diagnostics, therapies, reagents and kits, for modulating, eliciting, or detecting T cells responsive to one or more coronavirus peptides or proteins. The proteins and peptides described herein comprise, consist of, or consist essentially of: one or more amino acid sequences selected from those sequences set forth in Table 2 (SEQ ID NOS: 1 to 292) or Table 3 (SEQ ID NOS: 293 to 347), or a subsequence, portion, homologue, variant or derivative thereof; a fusion protein comprising one or more amino acid sequences selected from those sequences set forth in Table 2 or Table 3; a pool of 2 or more peptides selected from the amino acid sequences set forth in Table 2 or Table 3, or a polynucleotide that encodes one or more peptides or proteins, comprising, consisting of, or consisting essentially of an amino acid sequence selected from those sequences set forth in Table 2 or Table 3, or a subsequence, portion, homologue, variant or derivative thereof. In certain preferred embodiments, the coronavirus is one or more of SARS-CoV-2 or a variant thereof, or SARS, MERS, or a common cold coronavirus strain (e.g., 229E, NL63, HKU1, OC43). Further description and embodiments of such methods and compositions are provided in the definitions provided herein, and a person skilled in the art will recognize that the methods and compositions can be embodied in numerous variations, changes, and substitutions or as may occur to or be understood by one skilled in the art without departing from the disclosure.

The present inventors recognized that defining a comprehensive set of epitope specificities is important for several reasons. First, it allows the determination of whether within different SARS-CoV-2 antigens certain regions are immunodominant. This will be important for vaccine design, so as to ensure that vaccine constructs include not only regions targeted by neutralizing antibodies, such as the receptor binding domain (RBD) in the spike (S) region, but also include regions capable of delivering sufficient T cell help and are suitable targets of CD4+ T cell activity. Second, a comprehensive set of epitopes helps define the breadth of responses, in terms of the average number of different CD4+ and CD8+ T cell SARS-CoV-2 epitopes generally recognized by each individual. This is key because some reports have described a T cell repertoire focused on few viral epitopes (Ferretti et al., 2020), which would be concerning for potential viral escape from immune recognition via accumulated mutations that can occur during replication or through viral reassortment. Third, a comprehensive survey of epitopes restricted by a set of different HLAs representative of the diversity present in the general population is important to ensure that results obtained are generally applicable across different ethnicities and racial groups, and also to lay the foundations to examine the potential associations of certain HLAs with COVID-19 severity. Finally, the definition of the epitopes recognized in SARS-CoV-2 infection is relevant in the context of the debate on the potential influence of SARS-CoV-2 cross-reactivity with endemic “Common Cold” Coronaviruses (CCC) (Braun et al., 2020; Le Bert et al., 2020). Several studies have defined the repertoire of SARS-CoV-2 epitopes recognized in unexposed individuals (Braun et al., 2020; Mateus et al., 2020; Nelde et al., 2020), but the correspondence between that repertoire and the epitope repertoire elicited by SARS-CoV-2 infection has not been previously evaluated.

The present inventors provide a comprehensive map of epitopes recognized by CD4+ and CD8+ T cell responses across the entire SARS-CoV-2 viral proteome. Importantly, these epitopes have been characterized in the context of a broad set of HLA alleles using a direct ex vivo, cytokine-independent, approach.

The present inventors used a combined experimental and bioinformatics approach to address T cell reactivity to SARS-CoV-2 VOCs. T cell responses from persons recovered from COVID-19 were directly assessed, and T cell responses from recent Moderna mRNA-1273 or Pfizer/BioNTech BNT162b2 vaccinees, for their capacity to recognize peptides derived from the ancestral reference sequence and the B.1.1.7, B1.351, P.1 and CAL.20C variants. As a complementary approach, bioinformatic analyses were used to predict the impact of mutations in the VOCs with sets of previously reported CD4⁺ and CD8⁺ T cell epitopes derived from the ancestral reference sequence.

Sequence analysis and peptide pool generation. As a first step, the inventors mapped the specific mutations (amino acid replacements and deletions) of the main current VOCs, including B.1.1.7, B1.351, P.1 and CAL.20C, as compared to the SARS-CoV-2 Wuhan ancestral sequence (NCBI acc. no. NC_045512.2)(Table 1).

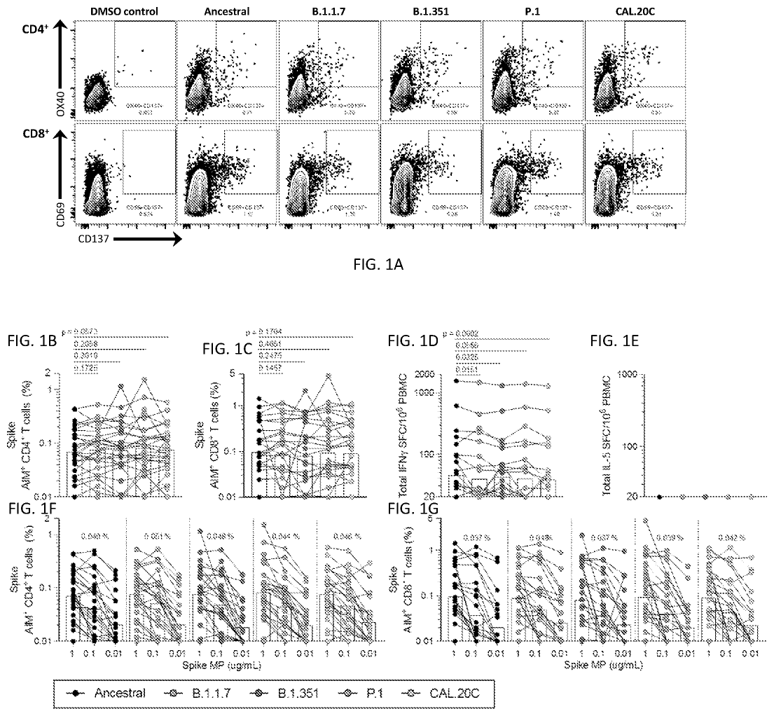

TABLE 1
Related to FIGS. 1, 2, 3 and 4. List of amino acid positions
and relative amino acid changes in the different variants
studied with respect to the ancestral Wuhan strain.

| | Amino acid | Ancestral | B.1.1.7 | B.1.351 | P.1. | CAL.20C |
|---|---|---|---|---|---|---|
| Protein | position | (Wu) | (UK) | (SA) | (BR) | (CA) |
| S | 13 | S | | | | I |
| S | 18 | L | | F | F | |
| S | 20 | T | | | N | |
| S | 26 | P | | | S | |
| S | 69 | H | Del | | | |
| S | 70 | V | Del | | | |
| S | 80 | D | | A | | |
| S | 138 | D | | | Y | |
| S | 145 | Y | Del | | | |
| S | 152 | W | | | | C |
| S | 190 | R | | | S | |
| S | 215 | D | | G/H | | |
| S | 241 | L | | Del | | |
| S | 242 | L | | Del | | |
| S | 243 | A | | Del | | |
| S | 417 | K | | N | T | |
| S | 452 | L | | | | R |
| S | 484 | E | | K | K | |
| S | 501 | N | Y | Y | Y | |
| S | 570 | A | D | | | |
| S | 614 | D | G | G | G | G |
| S | 655 | H | | | Y | |
| S | 681 | P | H | | | |
| S | 701 | A | | V | | |
| S | 716 | T | I | | | |
| S | 938 | L | | | | F |
| S | 982 | S | A | | | |
| S | 1027 | T | | | I | |
| S | 1118 | D | H | | | |
| S | 1176 | V | | | F | |
| S | 1191 | K | | | | N |
| M | 162 | K | | N | | |
| N | 3 | D | L | | | |
| N | 13 | P | | S | | |
| N | 32 | R | | H | | |
| N | 80 | P | | | R | |
| N | 203 | R | K | | K | K |
| N | 204 | G | R | | R | R |
| N | 205 | T | | I | | I |
| N | 212 | G | | C | | |
| N | 234 | M | | | | I |
| N | 235 | S | F | | | |
| E | 71 | P | | L | | |
| ORF3a | 57 | Q | | H | | H |
| ORF3a | 131 | W | | L | | |
| ORF3a | 171 | S | | L | | |
| ORF3a | 253 | S | | | P | |
| ORF7a | 93 | V | | F | | |
| ORF8 | 27 | — | Stop | | | |
| ORF8 | 92 | E | | | K | |
| ORF8 | 121 | I | | L | | |
| nsp1 | 109 | P | | S | | |
| nsp2 | 85 | T | | I | | I |
| nsp2 | 339 | G | | S | | |
| nsp2 | 366 | S | | T | | |
| nsp2 | 427 | Q | | H | | |
| nsp2 | 563 | E | | D | | |
| nsp3 | 183 | T | I | | | |
| nsp3 | 186 | T | | | A | |
| nsp3 | 370 | S | | | L | |
| nsp3 | 778 | P | | | | S |
| nsp3 | 837 | K | | N | | |
| nsp3 | 890 | A | D | | | |
| nsp3 | 926 | C | | S | | |
| nsp3 | 977 | K | | | Q | |
| nsp3 | 1180 | T | | I | | |
| nsp3 | 1412 | I | T | | | |
| nsp3 | 1778 | N | | S | | |
| nsp4 | 395 | S | | | | T |
| nsp5 | 90 | K | | R | | |
| nsp5 | 193 | A | | V | | |
| nsp6 | 106 | S | Del | Del | Del | |
| nsp6 | 107 | G | Del | Del | Del | |
| nsp6 | 108 | F | Del | Del | Del | |
| nsp6 | 125 | L | | | | F |
| nsp6 | 135 | G | | S | | |
| nsp6 | 149 | V | | F | | |
| nsp6 | 167 | L | | | | F |
| nsp9 | 65 | I | | | | V |
| nsp10 | 105 | N | | K | | |
| nsp12 | 323 | P | L | L | L | L |
| nsp13 | 53 | P | | | | L |
| nsp13 | 209 | V | | | | F |
| nsp13 | 260 | D | | | | Y |
| nsp13 | 341 | E | | | D | |
| nsp13 | 588 | T | | I | | |
| nsp14 | 177 | L | | F | | |
| nsp14 | 326 | F | | | | L |
| nsp14 | 328 | V | | F | | |
| nsp15 | 91 | D | | | | Y |

Then, corresponding peptides associated with the different variants were synthesized and generated new peptide pools (megapools; MP) spanning the full genome sequences of the ancestral Wuhan strain and the respective B.1.1.7, B1.351, P.1 and CAL.20C variants the inventors (Table 2). As described below, the resulting peptide pools were assessed for their capacity to be recognized by memory T cells responses derived from natural infection in convalescents and vaccinees, and responses to the variant and ancestral genome antigen-specific pools were compared.

TABLE 2
Related to FIGS. 1, 2, 3 and 4. List of mutated peptides with respect
to the ancestral Wuhan strain in the different variants studied.

| | Amino acid | Amino acid | | | |
|---|---|---|---|---|---|
| | position | position | SARS-COV-2 | | SEQ ID |
| Protein | (Start) | (End) | strain | Sequence | NO: |
| nsp3 | 173 | 187 | B.1.1.7 | QDGSEDNQTTIIQTI | 1 |
| nsp3 | 178 | 192 | B.1.1.7 | DNQTTIIQTIVEVQP | 2 |
| nsp3 | 183 | 197 | B.1.1.7 | IIQTIVEVQPQLEME | 3 |
| nsp3 | 878 | 892 | B.1.1.7 | QDAYYRARAGEADNF | 4 |
| nsp3 | 883 | 897 | B.1.1.7 | RARAGEADNFCALIL | 5 |
| nsp3 | 888 | 902 | B.1.1.7 | EADNFCALILAYCNK | 6 |
| nsp3 | 1398 | 1412 | B.1.1.7 | NYLKSPNFSKLINIT | 7 |
| nsp3 | 1403 | 1417 | B.1.1.7 | PNFSKLINITIWFLL | 8 |
| nsp3 | 1408 | 1422 | B.1.1.7 | LINITIWFLLLSVCL | 9 |
| nsp6 | 92 | 106 | B.1.1.7 | MRIMTWLDMVDTSLK | 10 |
| nsp6 | 97 | 111 | B.1.1.7 | WLDMVDTSLKLKDCV | 11 |
| nsp6 | 102 | 116 | B.1.1.7 | DTSLKLKDCVMYASA | 12 |
| nsp6 | 107 | 121 | B.1.1.7 | KLKDCVMYASAVVLL | 13 |
| nsp12 | 309 | 323 | B.1.1.7 | HCANFNVLFSTVFPL | 14 |
| nsp12 | 314 | 328 | B.1.1.7 | NVLFSTVFPLTSFGP | 15 |
| nsp12 | 319 | 333 | B.1.1.7 | TVFPLTSFGPLVRKI | 16 |
| N | 1 | 15 | B.1.1.7 | MSLNGPQNQRNAPRI | 17 |
| N | 191 | 205 | B.1.1.7 | RNSSRNSTPGSSKRT | 18 |
| N | 196 | 210 | B.1.1.7 | NSTPGSSKRTSPARM | 19 |
| N | 201 | 215 | B.1.1.7 | SSKRTSPARMAGNGG | 20 |
| N | 221 | 235 | B.1.1.7 | LLLLDRLNQLESKMF | 21 |
| N | 226 | 240 | B.1.1.7 | RLNQLESKMFGKGQQ | 22 |
| N | 231 | 245 | B.1.1.7 | ESKMFGKGQQQQGQT | 23 |
| ORF8 | 41 | 55 | B.1.1.7 | FYSKWYIRVGAIKSA | 24 |
| ORF8 | 46 | 60 | B.1.1.7 | YIRVGAIKSAPLIEL | 25 |
| ORF8 | 51 | 65 | B.1.1.7 | AIKSAPLIELCVDEA | 26 |
| ORF8 | 61 | 75 | B.1.1.7 | CVDEAGSKSPIQCID | 27 |
| ORF8 | 66 | 80 | B.1.1.7 | GSKSPIQCIDIGNYT | 28 |
| ORF8 | 71 | 85 | B.1.1.7 | IQCIDIGNYTVSCLP | 29 |
| S | 56 | 70 | B.1.1.7 | LPFFSNVTWFHAISG | 30 |
| S | 61 | 75 | B.1.1.7 | NVTWFHAISGTNGTK | 31 |
| S | 66 | 80 | B.1.1.7 | HAISGTNGTKRFDNP | 32 |
| S | 131 | 145 | B.1.1.7 | CEFQFCNDPFLGVYH | 33 |
| S | 136 | 150 | B.1.1.7 | CNDPFLGVYHKNNKS | 34 |
| S | 141 | 155 | B.1.1.7 | LGVYHKNNKSWMESE | 35 |
| S | 491 | 505 | B.1.1.7 | PLQSYGFQPTYGVGY | 36 |
| S | 496 | 510 | B.1.1.7 | GFQPTYGVGYQPYRV | 37 |
| S | 501 | 515 | B.1.1.7 | YGVGYQPYRVVVLSF | 38 |
| S | 556 | 570 | B.1.1.7 | NKKFLPFQQFGRDID | 39 |
| S | 561 | 575 | B.1.1.7 | PFQQFGRDIDDTTDA | 40 |
| S | 566 | 580 | B.1.1.7 | GRDIDDTTDAVRDPQ | 41 |
| S | 601 | 615 | B.1.1.7 | GTNTSNQVAVLYQGV | 42 |
| S | 606 | 620 | B.1.1.7 | NQVAVLYQGVNCTEV | 43 |
| S | 611 | 625 | B.1.1.7 | LYQGVNCTEVPVAIH | 44 |
| S | 671 | 685 | B.1.1.7 | CASYQTQTNSHRRAR | 45 |
| S | 676 | 690 | B.1.1.7 | TQTNSHRRARSVASQ | 46 |
| S | 681 | 695 | B.1.1.7 | HRRARSVASQSIIAY | 47 |
| S | 706 | 720 | B.1.1.7 | AYSNNSIAIPINFTI | 48 |
| S | 711 | 725 | B.1.1.7 | SIAIPINFTISVTTE | 49 |
| S | 716 | 730 | B.1.1.7 | INFTISVTTEILPVS | 50 |
| S | 971 | 985 | B.1.1.7 | GAISSVLNDILARLD | 51 |
| S | 976 | 990 | B.1.1.7 | VLNDILARLDKVEAE | 52 |
| S | 981 | 995 | B.1.1.7 | LARLDKVEAEVQIDR | 53 |
| S | 1106 | 1120 | B.1.1.7 | QRNFYEPQIITTHNT | 54 |
| S | 1111 | 1125 | B.1.1.7 | EPQIITTHNTFVSGN | 55 |
| S | 1116 | 1130 | B.1.1.7 | TTHNTFVSGNCDVVI | 56 |
| N | 1 | 15 | B.1.351 | MSDNGPQNQRNASRI | 57 |
| N | 6 | 20 | B.1.351 | PQNQRNASRITFGGP | 58 |
| N | 11 | 25 | B.1.351 | NASRITFGGPSDSTG | 59 |
| N | 21 | 35 | B.1.351 | SDSTGSNQNGEHSGA | 60 |
| N | 26 | 40 | B.1.351 | SNQNGEHSGARSKQR | 61 |
| N | 31 | 45 | B.1.351 | EHSGARSKQRRPQGL | 62 |
| N | 191 | 205 | B.1.351 | RNSSRNSTPGSSRGI | 63 |
| N | 196 | 210 | B.1.351 | NSTPGSSRGISPARM | 64 |
| N | 201 | 215 | B.1.351 | SSRGISPARMACNGG | 65 |
| N | 206 | 220 | B.1.351 | SPARMACNGGDAALA | 66 |
| N | 211 | 225 | B.1.351 | ACNGGDAALALLLLD | 67 |
| ORF8 | 107 | 121 | B.1.351 | DFLEYHDVRVVLDFL | 68 |
| ORF7a | 81 | 95 | B.1.351 | SVSPKLFIRQEEFQE | 69 |
| ORF7a | 86 | 100 | B.1.351 | LFIRQEEFQELYSPI | 70 |
| ORF7a | 91 | 105 | B.1.351 | EEFQELYSPIFLIVA | 71 |
| M | 151 | 165 | B.1.351 | IAGHHLGRCDINDLP | 72 |
| M | 156 | 170 | B.1.351 | LGRCDINDLPKEITV | 73 |
| M | 161 | 175 | B.1.351 | INDLPKEITVATSRT | 74 |
| ORF3a | 46 | 60 | B.1.351 | LIVGVALLAVFHSAS | 75 |
| ORF3a | 51 | 65 | B.1.351 | ALLAVFHSASKIITL | 76 |
| ORF3a | 56 | 70 | B.1.351 | FHSASKIITLKKRWQ | 77 |
| ORF3a | 121 | 135 | B.1.351 | VRIIMRLWLCLKCRS | 78 |
| ORF3a | 126 | 140 | B.1.351 | RLWLCLKCRSKNPLL | 79 |
| ORF3a | 131 | 145 | B.1.351 | LKCRSKNPLLYDANY | 80 |
| ORF3a | 161 | 175 | B.1.351 | NSVTSSIVITLGDGT | 81 |
| ORF3a | 166 | 180 | B.1.351 | SIVITLGDGTTSPIS | 82 |
| ORF3a | 171 | 185 | B.1.351 | LGDGTTSPISEHDYQ | 83 |
| nsp1 | 96 | 110 | B.1.351 | QYGRSGETLGVLVSH | 84 |
| nsp1 | 101 | 115 | B.1.351 | GETLGVLVSHVGEIP | 85 |
| nsp1 | 106 | 120 | B.1.351 | VLVSHVGEIPVAYRK | 86 |
| nsp5 | 183 | 197 | B.1.351 | GPFVDRQTAQVAGTD | 87 |
| nsp5 | 188 | 202 | B.1.351 | RQTAQVAGTDTTITV | 88 |
| nsp5 | 193 | 207 | B.1.351 | VAGTDTTITVNVLAW | 89 |
| nsp10 | 93 | 107 | B.1.351 | KGKYVQIPTTCAKDP | 90 |
| nsp10 | 98 | 112 | B.1.351 | QIPTTCAKDPVGFTL | 91 |
| nsp10 | 103 | 117 | B.1.351 | CAKDPVGFTLKNTVC | 92 |
| nsp12 | 309 | 323 | B.1.351 | HCANFNVLFSTVFPL | 93 |
| nsp12 | 314 | 328 | B.1.351 | NVLFSTVFPLTSFGP | 94 |
| nsp12 | 319 | 333 | B.1.351 | TVFPLTSFGPLVRKI | 95 |
| nsp14 | 316 | 330 | B.1.351 | VVKAALLADKFPFLH | 96 |
| nsp14 | 321 | 335 | B.1.351 | LLADKFPFLHDIGNP | 97 |
| nsp14 | 326 | 340 | B.1.351 | FPFLHDIGNPKAIKC | 98 |
| S | 6 | 20 | B.1.351 | VLLPLVSSQCVNFTT | 99 |
| S | 11 | 25 | B.1.351 | VSSQCVNFTTRTQLP | 100 |
| S | 16 | 30 | B.1.351 | VNFTTRTQLPPAYTN | 101 |
| S | 66 | 80 | B.1.351 | HAIHVSGTNGTKRFA | 102 |
| S | 71 | 85 | B.1.351 | SGTNGTKRFANPVLP | 103 |
| S | 76 | 90 | B.1.351 | TKRFANPVLPFNDGV | 104 |
| S | 201 | 215 | B.1.351 | FKIYSKHTPINLVRH | 105 |
| S | 201 | 215 | B.1.351 | FKIYSKHTPINLVRG | 106 |
| S | 206 | 220 | B.1.351 | KHTPINLVRHLPQGF | 107 |
| S | 206 | 220 | B.1.351 | KHTPINLVRGLPQGF | 108 |
| S | 211 | 225 | B.1.351 | NLVRHLPQGFSALEP | 109 |
| S | 211 | 225 | B.1.351 | NLVRGLPQGFSALEP | 110 |
| E | 61 | 75 | B.1.351 | RVKNLNSSRVLDLLV | 111 |
| nsp2 | 71 | 85 | B.1.351 | LQTPFEIKLAKKFDI | 112 |
| nsp2 | 76 | 90 | B.1.351 | EIKLAKKFDIFNGEC | 113 |
| nsp2 | 81 | 95 | B.1.351 | KKFDIFNGECPNFVF | 114 |
| nsp2 | 326 | 340 | B.1.351 | CGNFKVTKGKAKKSA | 115 |
| nsp2 | 331 | 345 | B.1.351 | VTKGKAKKSAWNIGE | 116 |
| nsp2 | 336 | 350 | B.1.351 | AKKSAWNIGEQKSIL | 117 |
| nsp2 | 356 | 370 | B.1.351 | FASEAARVVRTIFSR | 118 |
| nsp2 | 361 | 375 | B.1.351 | ARVVRTIFSRTLETA | 119 |
| nsp2 | 366 | 380 | B.1.351 | TIFSRTLETAQNSVR | 120 |
| nsp2 | 416 | 430 | B.1.351 | VVMAYITGGVVHLTS | 121 |
| nsp2 | 421 | 435 | B.1.351 | ITGGVVHLTSQWLTN | 122 |
| nsp2 | 426 | 440 | B.1.351 | VHLTSQWLTNIFGTV | 123 |
| nsp2 | 551 | 565 | B.1.351 | MPLKAPKEIIFLDGE | 124 |
| nsp2 | 556 | 570 | B.1.351 | PKEIIFLDGETLPTE | 125 |
| nsp2 | 561 | 575 | B.1.351 | FLDGETLPTEVLTEE | 126 |
| nsp3 | 823 | 837 | B.1.351 | SFLGRYMSALNHTKN | 127 |
| nsp3 | 828 | 842 | B.1.351 | YMSALNHTKNWKYPQ | 128 |
| nsp3 | 833 | 847 | B.1.351 | NHTKNWKYPQVNGLT | 129 |
| nsp3 | 1168 | 1182 | B.1.351 | LHKPIVWHVNNAINK | 130 |
| nsp3 | 1173 | 1187 | B.1.351 | VWHVNNAINKATYKP | 131 |
| nsp3 | 1178 | 1192 | B.1.351 | NAINKATYKPNTWCI | 132 |
| nsp3 | 1768 | 1782 | B.1.351 | VAVKMFDAYVSTFSS | 133 |
| nsp3 | 1773 | 1787 | B.1.351 | FDAYVSTFSSTFNVP | 134 |
| nsp3 | 1778 | 1792 | B.1.351 | STFSSTFNVPMEKLK | 135 |
| nsp13 | 577 | 591 | B.1.351 | SDRDLYDKLQFISLE | 136 |
| nsp13 | 582 | 596 | B.1.351 | YDKLQFISLEIPRRN | 137 |
| nsp13 | 587 | 601 | B.1.351 | FISLEIPRRNVATLQ | 138 |
| nsp6 | 92 | 106 | B.1.351 | MRIMTWLDMVDTSLK | 139 |
| nsp6 | 97 | 111 | B.1.351 | WLDMVDTSLKLKDCV | 140 |
| nsp6 | 102 | 116 | B.1.351 | DTSLKLKDCVMYASA | 141 |
| nsp6 | 107 | 121 | B.1.351 | LKDCVMYASAVVLLI | 142 |
| nsp6 | 122 | 136 | B.1.351 | LLILMTARTVYDDSA | 143 |
| nsp6 | 127 | 141 | B.1.351 | TARTVYDDSARRVWT | 144 |
| nsp6 | 132 | 146 | B.1.351 | YDDSARRVWTLMNVL | 145 |
| nsp6 | 137 | 151 | B.1.351 | RRVWTLMNVLTLFYK | 146 |
| nsp6 | 142 | 156 | B.1.351 | LMNVLTLFYKVYYGN | 147 |
| nsp6 | 147 | 161 | B.1.351 | TLFYKVYYGNALDQA | 148 |
| S | 231 | 245 | B.1.351 | IGINITRFQTLHRSY | 149 |
| S | 236 | 250 | B.1.351 | TRFQTLHRSYLTPGD | 150 |
| S | 241 | 255 | B.1.351 | LHRSYLTPGDSSSGW | 151 |
| S | 406 | 420 | B.1.351 | EVRQIAPGQTGNIAD | 152 |
| S | 411 | 425 | B.1.351 | APGQTGNIADYNYKL | 153 |
| S | 416 | 430 | B.1.351 | GNIADYNYKLPDDFT | 154 |
| S | 476 | 490 | B.1.351 | EIYQAGSTPCNGVKG | 155 |
| S | 481 | 495 | B.1.351 | GSTPCNGVKGFNCYF | 156 |
| S | 486 | 500 | B.1.351 | NGVKGFNCYFPLQSY | 157 |
| S | 491 | 505 | B.1.351 | PLQSYGFQPTYGVGY | 158 |
| S | 496 | 510 | B.1.351 | GFQPTYGVGYQPYRV | 159 |
| S | 501 | 515 | B.1.351 | YGVGYQPYRVVVLSF | 160 |
| S | 606 | 620 | B.1.351 | GTNTSNQVAVLYQGV | 161 |
| S | 611 | 625 | B.1.351 | NQVAVLYQGVNCTEV | 162 |
| S | 616 | 630 | B.1.351 | LYQGVNCTEVPVAIH | 163 |
| S | 691 | 705 | B.1.351 | SIIAYTMSLGVENSV | 164 |
| S | 696 | 710 | B.1.351 | TMSLGVENSVAYSNN | 165 |
| S | 701 | 715 | B.1.351 | VENSVAYSNNSIAIP | 166 |
| N | 66 | 80 | P.1. | FPRGQGVPINTNSSR | 167 |
| N | 71 | 85 | P.1. | GVPINTNSSRDDQIG | 168 |
| N | 76 | 90 | P.1. | TNSSRDDQIGYYRRA | 169 |
| N | 191 | 205 | P.1. | RNSSRNSTPGSSKRT | 170 |
| N | 196 | 210 | P.1. | NSTPGSSKRTSPARM | 171 |
| N | 201 | 215 | P.1. | SSKRTSPARMAGNGG | 172 |
| ORF8 | 81 | 95 | P.1. | VSCLPFTINCQKPKL | 173 |
| ORF8 | 86 | 100 | P.1. | FTINCQKPKLGSLVV | 174 |
| ORF8 | 91 | 105 | P.1. | QKPKLGSLVVRCSFY | 175 |
| ORF3a | 241 | 255 | P.1. | EEHVQIHTIDGSPGV | 176 |
| ORF3a | 246 | 260 | P.1. | IHTIDGSPGVVNPVM | 177 |
| ORF3a | 251 | 265 | P.1. | GSPGVVNPVMEPIYD | 178 |
| nsp12 | 309 | 323 | P.1. | HCANFNVLFSTVFPL | 179 |
| nsp12 | 314 | 328 | P.1. | NVLFSTVFPLTSFGP | 180 |
| nsp12 | 319 | 333 | P.1. | TVFPLTSFGPLVRKI | 181 |
| nsp13 | 327 | 341 | P.1. | IDKCSRIIPARARVD | 182 |
| nsp13 | 332 | 346 | P.1. | RIIPARARVDCFDKF | 183 |
| nsp13 | 337 | 351 | P.1. | RARVDCFDKFKVNST | 184 |
| S | 6 | 20 | P.1. | VLLPLVSSQCVNFTN | 185 |
| S | 11 | 25 | P.1. | VSSQCVNFTNRTQLP | 186 |
| S | 16 | 30 | P.1. | VNFTNRTQLPSAYTN | 187 |
| S | 21 | 35 | P.1. | RTQLPSAYTNSFTRG | 188 |
| S | 26 | 40 | P.1. | SAYTNSFTRGVYYPD | 189 |
| S | 126 | 140 | P.1. | VVIKVCEFQFCNYPF | 190 |
| S | 131 | 145 | P.1. | CEFQFCNYPFLGVYY | 191 |
| S | 136 | 150 | P.1. | CNYPFLGVYYHKNNK | 192 |
| S | 176 | 190 | P.1. | LMDLEGKQGNFKNLS | 193 |
| S | 181 | 195 | P.1. | GKQGNFKNLSEFVFK | 194 |
| S | 186 | 200 | P.1. | FKNLSEFVFKNIDGY | 195 |
| S | 406 | 420 | P.1. | EVRQIAPGQTGTIAD | 196 |
| S | 411 | 425 | P.1. | APGQTGTIADYNYKL | 197 |
| S | 416 | 430 | P.1. | GTIADYNYKLPDDFT | 198 |
| S | 471 | 485 | P.1. | EIYQAGSTPCNGVKG | 199 |
| S | 476 | 490 | P.1. | GSTPCNGVKGFNCYF | 200 |
| S | 481 | 495 | P.1. | NGVKGFNCYFPLQSY | 201 |
| S | 491 | 505 | P.1. | PLQSYGFQPTYGVGY | 202 |
| S | 496 | 510 | P.1. | GFQPTYGVGYQPYRV | 203 |
| S | 501 | 515 | P.1. | YGVGYQPYRVVVLSF | 204 |
| S | 601 | 615 | P.1. | GTNTSNQVAVLYQGV | 205 |
| S | 606 | 620 | P.1. | NQVAVLYQGVNCTEV | 206 |
| S | 611 | 625 | P.1. | LYQGVNCTEVPVAIH | 207 |
| S | 641 | 655 | P.1. | NVFQTRAGCLIGAEY | 208 |
| S | 646 | 660 | P.1. | RAGCLIGAEYVNNSY | 209 |
| S | 651 | 665 | P.1. | IGAEYVNNSYECDIP | 210 |
| S | 1016 | 1030 | P.1. | AEIRASANLAAIKMS | 211 |
| S | 1021 | 1035 | P.1. | SANLAAIKMSECVLG | 212 |
| S | 1026 | 1040 | P.1. | AIKMSECVLGQSKRV | 213 |
| S | 1166 | 1180 | P.1. | LGDISGINASFVNIQ | 214 |
| S | 1171 | 1185 | P.1. | GINASFVNIQKEIDR | 215 |
| S | 1176 | 1190 | P.1. | FVNIQKEIDRLNEVA | 216 |
| nsp3 | 173 | 187 | P.1. | QDGSEDNQTTTIQAI | 217 |
| nsp3 | 178 | 192 | P.1. | DNQTTTIQAIVEVQP | 218 |
| nsp3 | 183 | 197 | P.1. | TIQAIVEVQPQLEME | 219 |
| nsp3 | 358 | 372 | P.1. | AVFDKNLYDKLVLSF | 220 |
| nsp3 | 363 | 377 | P.1. | NLYDKLVLSFLEMKS | 221 |
| nsp3 | 368 | 382 | P.1. | LVLSFLEMKSEKQVE | 222 |
| nsp3 | 963 | 977 | P.1. | KGVQIPCTCGKQATQ | 223 |
| nsp3 | 968 | 982 | P.1. | PCTCGKQATQYLVQQ | 224 |
| nsp3 | 973 | 987 | P.1. | KQATQYLVQQESPFV | 225 |
| nsp6 | 92 | 106 | P.1. | MRIMTWLDMVDTSLK | 226 |
| nsp6 | 97 | 111 | P.1. | WLDMVDTSLKLKDCV | 227 |
| nsp6 | 102 | 116 | P.1. | DTSLKLKDCVMYASA | 228 |
| nsp6 | 107 | 121 | P.1. | LKDCVMYASAVVLLI | 229 |
| N | 191 | 205 | CAL.20C | RNSSRNSTPGSSKRI | 230 |
| N | 196 | 210 | CAL.20C | NSTPGSSKRISPARM | 231 |
| N | 201 | 215 | CAL.20C | SSKRISPARMAGNGG | 232 |
| N | 221 | 235 | CAL.20C | LLLLDRLNQLESKIS | 233 |
| N | 226 | 240 | CAL.20C | RLNQLESKISGKGQQ | 234 |
| N | 231 | 245 | CAL.20C | ESKISGKGQQQQGQT | 235 |
| nsp2 | 71 | 85 | CAL.20C | LQTPFEIKLAKKFDI | 236 |
| nsp2 | 76 | 90 | CAL.20C | EIKLAKKFDIFNGEC | 237 |
| nsp2 | 81 | 95 | CAL.20C | KKFDIFNGECPNFVF | 238 |
| nsp3 | 768 | 782 | CAL.20C | MSMTYGQQFGSTYLD | 239 |
| nsp3 | 773 | 787 | CAL.20C | GQQFGSTYLDGADVT | 240 |
| nsp3 | 778 | 792 | CAL.20C | STYLDGADVTKIKPH | 241 |
| nsp4 | 383 | 397 | CAL.20C | ICISTKHFYWFFTNY | 242 |
| nsp4 | 388 | 402 | CAL.20C | KHFYWFFTNYLKRRV | 243 |
| nsp4 | 393 | 407 | CAL.20C | FFTNYLKRRVVFNGV | 244 |
| nsp6 | 112 | 126 | CAL.20C | DCVMYASAVVLLIFM | 245 |
| nsp6 | 117 | 131 | CAL.20C | ASAVVLLIFMTARTV | 246 |
| nsp6 | 122 | 136 | CAL.20C | LLIFMTARTVYDDGA | 247 |
| nsp6 | 157 | 171 | CAL.20C | ALDQAISMWAFIISV | 248 |
| nsp6 | 162 | 176 | CAL.20C | ISMWAFIISVTSNYS | 249 |
| nsp6 | 167 | 181 | CAL.20C | FIISVTSNYSGVVTT | 25 |
| nsp9 | 51 | 65 | CAL.20C | LKWARFPKSDGTGTV | 251 |
| nsp9 | 56 | 70 | CAL.20C | FPKSDGTGTVYTELE | 252 |
| nsp9 | 61 | 75 | CAL.20C | GTGTVYTELEPPCRF | 253 |
| nsp12 | 309 | 323 | CAL.20C | HCANFNVLFSTVFPL | 254 |
| nsp12 | 314 | 328 | CAL.20C | NVLFSTVFPLTSFGP | 255 |
| nsp12 | 319 | 333 | CAL.20C | TVFPLTSFGPLVRKI | 256 |
| nsp13 | 42 | 56 | CAL.20C | VLSVNPYVCNAPGCD | 257 |
| nsp13 | 47 | 61 | CAL.20C | PYVCNAPGCDVTDVT | 258 |
| nsp13 | 52 | 66 | CAL.20C | APGCDVTDVTQLYLG | 259 |
| nsp13 | 197 | 211 | CAL.20C | EYTFEKGDYGDAFVY | 260 |
| nsp13 | 202 | 216 | CAL.20C | KGDYGDAFVYRGTTT | 261 |
| nsp13 | 207 | 221 | CAL.20C | DAFVYRGTTTYKLNV | 262 |
| nsp13 | 247 | 261 | CAL.20C | VRITGLYPTLNISYE | 263 |
| nsp13 | 252 | 266 | CAL.20C | LYPTLNISYEFSSNV | 264 |
| nsp13 | 257 | 271 | CAL.20C | NISYEFSSNVANYQK | 265 |
| nsp14 | 316 | 330 | CAL.20C | VVKAALLADKLPVLH | 266 |
| nsp14 | 321 | 335 | CAL.20C | LLADKLPVLHDIGNP | 267 |
| nsp14 | 326 | 340 | CAL.20C | LPVLHDIGNPKAIKC | 268 |
| nsp15 | 79 | 93 | CAL.20C | IAANTVIWDYKRYAP | 269 |
| nsp15 | 84 | 98 | CAL.20C | VIWDYKRYAPAHIST | 270 |
| nsp15 | 89 | 103 | CAL.20C | KRYAPAHISTIGVCS | 271 |
| ORF3a | 46 | 60 | CAL.20C | LIVGVALLAVFHSAS | 272 |
| ORF3a | 51 | 65 | CAL.20C | ALLAVFHSASKIITL | 273 |
| ORF3a | 56 | 70 | CAL.20C | FHSASKIITLKKRWQ | 274 |
| S | 1 | 15 | CAL.20C | MFVFLVLLPLVSIQC | 275 |
| S | 6 | 20 | CAL.20C | VLLPLVSIQCVNLTT | 276 |
| S | 11 | 25 | CAL.20C | VSIQCVNLTTRTQLP | 277 |
| S | 141 | 155 | CAL.20C | LGVYYHKNNKSCMES | 278 |
| S | 146 | 160 | CAL.20C | HKNNKSCMESEFRVY | 279 |
| S | 151 | 165 | CAL.20C | SCMESEFRVYSSANN | 280 |
| S | 441 | 455 | CAL.20C | LDSKVGGNYNYRYRL | 281 |
| S | 446 | 460 | CAL.20C | GGNYNYRYRLFRKSN | 282 |
| S | 451 | 465 | CAL.20C | YRYRLFRKSNLKPFE | 283 |
| S | 601 | 615 | CAL.20C | GTNTSNQVAVLYQGV | 284 |
| S | 606 | 620 | CAL.20C | NQVAVLYQGVNCTEV | 285 |
| S | 611 | 625 | CAL.20C | LYQGVNCTEVPVAIH | 286 |
| S | 926 | 940 | CAL.20C | QFNSAIGKIQDSFSS | 287 |
| S | 931 | 945 | CAL.20C | IGKIQDSFSSTASAL | 288 |
| S | 936 | 950 | CAL.20C | DSFSSTASALGKLQD | 289 |
| S | 1181 | 1195 | CAL.20C | KEIDRLNEVANNLNE | 290 |
| S | 1186 | 1200 | CAL.20C | LNEVANNLNESLIDL | 291 |
| S | 1191 | 1205 | CAL.20C | NNLNESLIDLQELGK | 292 |

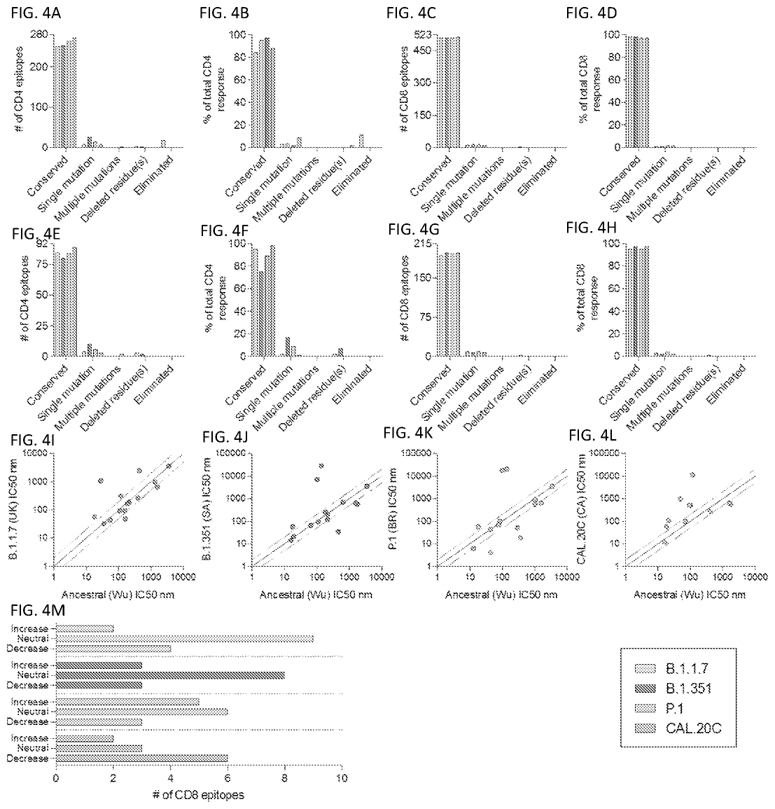

TABLE 3
Related to FIG. 4. Effect of mutations on CD8 epitope HLA class I binding capacity

| | | | | | | WT | Mutant | | | SEQ |
|---|---|---|---|---|---|---|---|---|---|---|
| | | | | | HLA | (IC₅₀ | (IC₅₀ | Fold | | ID |
| Origin | Protein | Start | Mutation^{a} | Mutated sequence^{b} | restriction | nM)^{c} | nM) | difference | Effect^{d} | NO: |
| B.1.1.7 | S | 69 | HV69-70 del | HAISGTNGTK | A*68:01 | 55 | 44 | 0.8 | Neutral | 293 |
| B.1.1.7 | S | 142 | Y145 del | FLGVYHKNNK | A*03:01 | 28 | 1078 | 39 | Decrease | 294 |
| B.1.1.7 | S | 144 | Y145 del | VYHKNNKSW | A*24:02 | 117 | 308 | 2.6 | Decrease | 295 |
| B.1.1.7 | S | 495 | N501Y | YGFQPTYGV | B*51:01 | 3488 | 3541 | 1.0 | Neutral | 296 |
| B.1.1.7 | S | 612 | D614G | YQGVNCTEV | A*02:06 | 18 | 57 | 3.2 | Decrease | 297 |
| B.1.1.7 | S | 677 | P681H | QTNSHRRAR | A*31:01 | 35 | 33 | 0.94 | Neutral | 298 |
| B.1.1.7 | S | 680 | P681H | SHRRARSV | B*08:01 | 429 | 2449 | 5.7 | Decrease | 299 |
| B.1.1.7 | S | 710 | T716I | NSIAIPINF | B*57:01 | 1335 | 968 | 0.73 | Neutral | 300 |
| B.1.1.7 | S | 712 | T716I | IAIPINFTI | B*51:01 | 209 | 189 | 0.90 | Neutral | 301 |
| B.1.1.7 | S | 712 | T716I | IAIPINFTI | B*53:01 | 396 | 266 | 0.67 | Neutral | 302 |
| B.1.1.7 | S | 714 | T716I | IPINFTISV | B*07:02 | 188 | 168 | 0.89 | Neutral | 303 |
| B.1.1.7 | S | 714 | T716I | IPINFTISV | B*51:01 | 156 | 94 | 0.60 | Neutral | 304 |
| B.1.1.7 | S | 975 | S982A | SVLNDILAR | A*68:01 | 109 | 92 | 0.84 | Neutral | 305 |
| B.1.1.7 | nsp3 | 1407 | I1412T | KLINITIWF | A*32:01 | 161 | 48 | 0.30 | Increase | 306 |
| B.1.1.7 | nsp12 | 318 | P323L | STVFPLTSF | B*57:01 | 1583 | 637 | 0.40 | Increase | 307 |
| B.1.351 | S | 78 | D80A | RFANPVLPF | A*24:02 | 458 | 34 | 0.075 | Increase | 308 |
| B.1.351 | S | 79 | D80A | FANPVLPFNDGVYF | B*35:01 | 65 | 65 | 1.0 | Neutral | 309 |
| B.1.351 | S | 208 | D215G | TPINLVRGL | B*07:02 | 213 | 119 | 0.56 | Neutral | 310 |
| B.1.351 | S | 208 | D215H | TPINLVRHL | B*07:02 | 213 | 199 | 0.93 | Neutral | 311 |
| B.1.351 | S | 409 | K417N | QIAPGQTGN | A*68:01 | 137 | 27998 | 204 | Decrease | 312 |
| B.1.351 | S | 495 | N501Y | YGFQPTYGV | B*51:01 | 3488 | 3541 | 1.0 | Neutral | 313 |
| B.1.351 | S | 612 | D614G | YQGVNCTEV | A*02:06 | 18 | 57 | 3.2 | Decrease | 314 |
| B.1.351 | S | 695 | A701V | YTMSLGVENSVAY | A*26:01 | 184 | 253 | 1.4 | Neutral | 315 |
| B.1.351 | S | 699 | A701V | LGVENSVAY | B*35:01 | 19 | 21 | 1.1 | Neutral | 316 |
| B.1.351 | N | 5 | K17N | GPQNQRNASRITF | B*07:02 | 640 | 696 | 1.1 | Neutral | 317 |
| B.1.351 | ORF3a | 57 | Q57H | HSASKIITL | B*08:01 | 1788 | 573 | 0.32 | Increase | 318 |
| B.1.351 | nsp3 | 829 | K837N | MSALNHTKN | A*30:01 | 102 | 7035 | 69 | Decrease | 319 |
| B.1.351 | nsp3 | 829 | K837N | MSALNHTKNW | B*57:01 | 16 | 14 | 0.88 | Neutral | 320 |
| B.1.351 | nsp3 | 830 | K837N | SALNHTKNW | B*57:01 | 111 | 93 | 0.84 | Neutral | 321 |
| B.1.351 | nsp12 | 318 | P323L | STVFPLTSF | B*57:01 | 1583 | 637 | 0.40 | Increase | 322 |
| P.1. | S | 24 | P26S | LPSAYTNSF | B*07:02 | 294 | 51 | 0.17 | Increase | 323 |
| P.1. | S | 24 | P26S | LPSAYTNSF | B*35:01 | 44 | 4.1 | 0.093 | Increase | 324 |
| P.1. | S | 24 | P26S | LPSAYTNSF | B*53:01 | 366 | 18 | 0.049 | Increase | 325 |
| P.1. | S | 409 | K417T | QIAPGQTGT | A*68:01 | 137 | 20478 | 149 | Decrease | 326 |
| P.1. | S | 495 | N501Y | YGFQPTYGV | B*51:01 | 3488 | 3541 | 1.0 | Neutral | 327 |
| P.1. | S | 612 | D614G | YQGVNCTEV | A*02:06 | 18 | 57 | 3.2 | Decrease | 328 |
| P.1. | S | 653 | H655Y | AEYVNNSY | B*44:02 | 1038 | 904 | 0.87 | Neutral | 329 |
| P.1. | S | 653 | H655Y | AEYVNNSY | B*44:03 | 1020 | 577 | 0.57 | Neutral | 330 |
| P.1. | S | 1019 | L1027I | RASANLAAIK | A*03:01 | 85 | 99 | 1.2 | Neutral | 331 |
| P.1. | S | 1173 | V1176F | NASFVNIQK | A*68:01 | 13 | 6.2 | 0.48 | Increase | 332 |
| P.1. | N | 75 | P80R | NTNSSRDDQIGYY | A*01:01 | 44 | 44 | 1.0 | Neutral | 333 |
| P.1. | N | 79 | P80R | SRDDQIGYY | B*35:01 | 101 | 17781 | 175 | Decrease | 334 |
| P.1. | nsp3 | 364 | S370L | LYDKLVLSF | A*24:02 | 77 | 70 | 0.91 | Neutral | 335 |
| P.1. | nsp12 | 318 | P323L | STVFPLTSF | B*57:01 | 1583 | 637 | 0.40 | Increase | 336 |
| CAL.20C | S | 8 | S13I | LPLVSIQCV | B*51:01 | 402 | 272 | 0.68 | Neutral | 337 |
| CAL.20C | S | 144 | W152C | YYHKNNKSC | A*24:02 | 117 | 11134 | 95 | Decrease | 338 |
| CAL.20C | S | 151 | W152C | SCMESEFRVY | A*29:02 | 49 | 980 | 20 | Decrease | 339 |
| CAL.20C | S | 444 | L452R | KVGGNYNYRY | A*29:02 | 101 | 505 | 5.0 | Decrease | 340 |
| CAL.20C | S | 445 | L452R | VGGNYNYRY | A*29:02 | 94 | 519 | 5.5 | Decrease | 341 |
| CAL.20C | S | 448 | L452R | NYNYRYRLF | A*24:02 | 21 | 108 | 5.1 | Decrease | 342 |
| CAL.20C | S | 449 | L452R | YNYRYRLFR | A*31:01 | 16 | 12 | 0.75 | Neutral | 343 |
| CAL.20C | S | 612 | D614G | YQGVNCTEV | A*02:06 | 18 | 57 | 3.2 | Decrease | 344 |
| CAL.20C | ORF3a | 57 | Q57H | HSASKIITL | B*08:01 | 1788 | 573 | 0.32 | Increase | 345 |
| CAL.20C | nsp4 | 392 | S395T | WFFTNYLKR | A*31:01 | 70 | 98 | 1.4 | Neutral | 346 |
| CAL.20C | nsp12 | 318 | P323L | STVFPLTSF | B*57:01 | 1583 | 637 | 0.40 | Increase | 347 |

ªMutation noted as ancestral residue-position-variant residue. Del refers to deletion of the corresponding residue.
^{b}For deletion mutants, the peptide sequence shown represents the variant encompassing the same region that has the highest predicted binding affinity for the corresponding restricting allele.
^{c}Indicates predicted IC₅₀ for the corresponding reported restricting allele. Predictions were performed using the NetMHCpan BA 4.1 algorithm, hosted by the IEDB.
^{d}Increase/decrease in affinity defined by a two-fold difference in predicted IC₅₀ nM.

Cohorts of COVID-19 convalescent, vaccinees and unexposed controls. The inventors selected three donor cohorts to investigate T cell reactivity against VOCs. These convalescent donors were adults ranging from 20 to 67 years of age (median 38); 43% were male and 57% female (Table 4). SARS-CoV-2 infection in these donors was determined by PCR-based testing during the acute phase of their infection, if available (46% of the cases), and/or seropositivity determined by plasma SARS-CoV-2 S protein RBD IgG ELISA. From these donors, PBMC samples were collected between May and October, 2020, a period when none of the VOCs analyzed were prevalent in the San Diego area, where the donations from convalescent donors were obtained. The convalescent donor samples reflect the local ethnic demographics (81% white, non-Hispanic). Peak of COVID-19 disease severity was representative of the distribution observed in the general population, with a prevalence of mild cases (86% of the cohort analyzed).

From vaccinated donors, the inventors collected PBMC after recent vaccination with the Moderna mRNA-1273 or the Pfizer/BioNTech BNT162b2 vaccines, approximately 14 days following second dose administration (Table 4). These donors ranged in age from 23 to 67 years (median 47); 31% were male and 69% were female. All vaccinees had RBD IgG titers indicative of vaccination.

TABLE 4
Characteristics of donor cohorts.

| | COVID-19 (n = 28) | Vaccinees (n = 29) | Unexposed (n = 23) |
|---|---|---|---|
| Age (years) | 20-67 [Median = 38, | 23-67 [Median = 47, | 21-82 [Median = 34, |
| | IQR = 32] | IQR = 36] | IQR = 24] |
| Gender | | | |
| Male (%) | 43% (12/28) | 31% (9/29) | 35% (8/23) |
| Female (%) | 57% (16/28) | 69% (20/29) | 56% (13/23) |
| Unknown (%) | 0% (0/28) | 0% (0/29) | 9% (2/23) |
| Sample Collection Date | May-October 2020 | January-March 2021 | May 2014-March 2018 |
| | | | (13/23) |
| | | | March-May 2020 |
| | | | (10/23) |
| SARS-CoV-2 PCR | Positive = 92% (12/13) | N/A | N/A |
| | Not tested = 54% (15/28) | | |
| S RBD IgG Positive | 96% (27/28) | 100% (29/29) | 0% (10/10, |
| | | | collected in 2020) |
| Peak disease Severity^{a} | | | |
| Mild | 86% (24/28) | N/A | N/A |
| Moderate | 14% (4/28) | | |
| Severe | 0% (0/28) | | |
| Critical | 0% (0/28) | | |
| Race-Ethnicity | | | |
| White- not Hispanic or Latino | 81% (23/28) | 52% (15/29) | 43% (10/23) |
| Hispanic or Latino | 4% (1/28) | 10% (3/29) | 9% (2/23) |
| Asian | 7% (2/28) | 38% (11/29) | 26% (6/23) |
| American Indian/Alaska Native | 0% (0/28) | 0% (0/29) | 0% (0/23) |
| Black or African American | 0% (0/28) | 0% (0/29) | 9% (2/23) |
| More than one race | 4% (1/28) | 0% (0/29) | 4% (1/23) |
| Not reported | 4% (1/28) | 0% (0/29) | 9% (2/23) |
| Days at Collection | 38-163 (28/28) | 13-30 (14/29) | N/A |
| | | Pfizer | |
| | [Median = 78, | 12-16 (15/29) | |
| | | Moderna | |
| | IQR = 50]^{b} | [Median = 14, | |
| | | IQR = 14]^{c} | |

^{a}According to WHO criteria.
^{b}Post Symptom Onset
^{c}2^{nd} dose of vaccination

PBMC from unexposed donors were collected between May 2014 and March 2018, or in the March to May 2020, period, and were seronegative for RBD IgG. The unexposed donors ranged from 21 to 82 years old (median 34); 35% were male and 56% female, while 9% were unknown (Table 4).

CD4⁺ and CD8⁺ T cell reactivity against ancestral Spike. The inventors have previously described the use of Activation Induced Marker (AIM) assays to measure CD4⁺ and CD8⁺ T cell responses to pools of overlapping peptides spanning SARS-CoV-2 antigens^{34,39,47,48}. Here, the inventors utilized the same AIM techniques using OX40⁺CD137⁺ and CD69⁺CD137⁺ markers for CD4⁺ and CD8⁺ T cell reactivity, respectively.

The CD4⁺ and CD8⁺ T cell reactivity of the three cohorts against SARS-CoV-2 S by AIM and FluoroSPOT assays were compared. The T cell responses to the ancestral S MP were significantly higher than the DMSO controls in unexposed (p<0.0001 for CD4, p=0.0063 for CD8, and p=0.0036 for SFC/10⁶ PBMCs by the Wilcoxon Test), convalescent (p<0.0001 for CD4, p<0.0001 for CD8, and p=0.0008 for SFC/10⁶ PBMCs by the Wilcoxon Test) and vaccinated donors (p<0.0001 for CD4, p<0.0001 for CD8, and p<0.0001 for SFC/10⁶ PBMCs by the Wilcoxon Test).

When the AIM responses in the different donor cohorts were compared, a significantly higher reactivity in COVID-19 convalescents were found compared to unexposed individuals (CD4: p<0.0001; CD8: p<0.0001 by Mann-Whitney test), with 93% and 39% of donors positive (responses above the threshold of positivity as described in the Methods) for CD4⁺ and CD8⁺ T cell responses, respectively. These rates of positivity are similar to what was observed in a previous study analyzing responses to peptides spanning the whole SARS-CoV-2 proteome over a similar range of post symptom onset (PSO) days³⁹, where 93% and 50% positivity rates were observed for CD4⁺ and CD8⁺ T cells, respectively. Likewise, the responses observed to the S MP in vaccinated donors were significantly higher than in unexposed donors (CD4: p<0.0001; CD8: p<0.0001 by Mann-Whitney test), with 100% and 48% of donors positive for CD4 and CD8 responses, respectively.

With an IFNγ FluoroSPOT assay, IFNγ T cell responses were detected in 17%, 50% and 76% of unexposed, convalescent and vaccinated donors, respectively (Unexposed Vs Convalescent: p=0.019; Unexposed Vs Vaccinees: p<0.0001 by Mann-Whitney test). These donor T cell responses provided the benchmarks for subsequent assessment of the impact of VOC mutations.

CD4⁺ and CD8⁺ T cell reactivity against variants of concern (VOC) Spike protein. The inventors measured the CD4⁺ and CD8⁺ T cell responses to S MPs derived from the ancestral strain and corresponding MPs representing the B.1.1.7, B1.351, P.1 and CAL.20C variants. As shown in FIGS. 1A-C, good CD4⁺ and CD8⁺ T cell reactivity was observed in convalescent donors with peptides spanning the S protein of the ancestral Wuhan sequence and the corresponding variant S peptides. Geomean S-specific responses ranged from 0.07 to 0.08 for CD4⁺ T cells, and 0.08 to 0.10 for CD8⁺ T cells. No significant difference was observed between the ancestral and VOC S peptide pools (CD4: B.1.1.7 p=0.17; B.1.351 p=0.30; P.1 p=0.21; CAL.20C p=0.06 and CD8: B.1.1.7 p=0.15; B.1.351 p=0.25; P.1 p=0.47; CAL.20C p=0.17 by Wilcoxon test) (FIGS. 1A-C). These values (here and in subsequent graphs) are not corrected for multiple comparisons, as a correction would decrease statistical power for detecting significant differences; therefore, not performing multiple comparison corrections is the more conservative and stringent test.

To further test for potential differences in the recognition of VOCs by these T cells, the inventors calculated fold change values per individual donor. The inventors then performed a non-inferiority test on one sample Wilcoxon Signed Rank test compared to the lower bound fold change threshold calculated based on technical repeats (Table 3). No significant differences were observed, demonstrating that the decreases observed were within the range expected from technical repeat variation.

The VOC T cell analyses were extended using FluoroSPOT to measure the capacity of the various SARS-CoV-2 peptide pools to elicit functional responses in terms of secretion of IFNγ and IL-5 cytokines (FIGS. 1D-E). The results from the FluoroSPOT assay (FIGS. 1D-E) showed ex vivo IFNγ reactivity using whole PBMCs, with a geomean of 45 SFC/10⁶ PBMCs (range 20 to 1578) for the ancestral strain peptides. This overlaps with the 0-800 SFC range (median of 110) detected in a previous study describing Spike-reactivity in symptomatic donors⁴⁹, and with the 20 to 110 range for 2-5 months symptomatic donors reported in a separate study⁵⁰.

VOC reactivity was observed for the S MPs in convalescent donors, with geomean IFNγ SFC per million PBMCs ranging from 38 to 45 (FIG. 1D). Compared to the ancestral strain, there were significant decreases of 12%, 6%, and 14% for the B.1.1.7, B.1.351 and CAL.20C variant pools (B.1.1.7 p=0.02; B.1.351 p=0.03; P.1 p=0.07 and CAL.20C p<0.01 by Wilcoxon test), while no difference was observed for P.1 (FIG. 1D). No significant differences were observed by fold change analysis suggesting that the decreases observed were still within the technical fluctuation range. No IL-5 reactivity was observed for any of the pools (FIG. 1E).

To further assess the functionality of T cell recognition of these variants, the inventors considered the variant peptide dose response curves of the CD4⁺ and CD8⁺ T cells. As shown in FIG. 1F, peptide concentration sensitivity of CD4⁺ T cell responses to the ancestral and four variant pools were similar. The same pattern was also observed for CD8⁺ T cell responses to S (FIG. 1G).

CD4⁺ and CD8⁺ T cell total reactivity against variants of concern (VOC). As shown in Table 4, mutations found in the variants studied herein were not limited to the S protein, but also occurred in several additional antigens encoded in the SARS-CoV-2 genome. To address the potential impact of non-S variant mutations on overall proteome-wide CD4⁺ and CD8⁺ T cell reactivity, the inventors tested overlapping peptide MPs spanning the entire proteome of the ancestral Wuhan sequence in comparison with corresponding MPs representing the different variants.

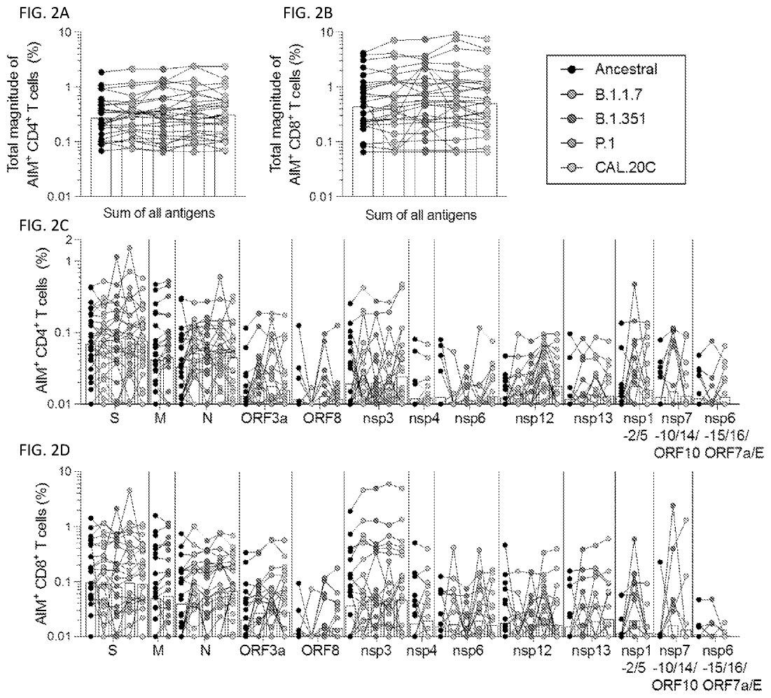

Overall, reactivity to the peptide pools spanning the variant genomes was found to be similar to that against the ancestral Wuhan strain (FIGS. 2A to 2G). When the sum total of reactivity throughout the genome was considered, no decreases in reactivity compared to the ancestral were noted for the variant pools (FIG. 2A-C).

The inventors previously showed in COVID-19 convalescent subjects that a set of 10 different antigens (nsp3, nsp4, nsp6, nsp12, nsp13, S, ORF3a, M, ORF8 and N) account for 83 and 81% of the total CD4⁺ and CD8⁺ T cell response, respectively⁵¹. Here, a similar overall pattern of dominant antigens was observed (FIG. 2C-D). For comparison purposes, unexposed donors were also tested in the AIM assay with MPs encompassing the ancestral and variant strains. As expected, some T cell reactivity against the SARS-CoV-2 MPs was observed in unexposed donors, possibly due to previous exposure to common cold coronaviruses^{47,48,52,53} or to other pathogens or autoantigens. The magnitude of the responses was lower than in COVID-19 convalescents (Unexposed vs. Convalescent: CD4 p<0.0001; CD8 p<0.0001 by Mann-Whitney test, comparison not shown in the graphs). Similar to the COVID-19 convalescents, no decrease in the geomean of AIM+CD4⁺ or CD8⁺ T cell responses to the sum of all antigens of individual MPs tested was observed in unexposed donors. Taken together, these experiments demonstrate that CD4⁺ and CD8⁺ T cells from individuals infected with the ancestral SARS-CoV-2 strain recognize VOCs with similar efficiency.

CD4⁺ and CD8⁺ T cell reactivity against VOCs by vaccinees. Next, the inventors studied T cell responses by individuals who received FDA authorized COVID-19 mRNA vaccines. The inventors focused the analysis on T cell reactivity to the S antigen of the ancestral strain, which is the basis of the presently used vaccines.

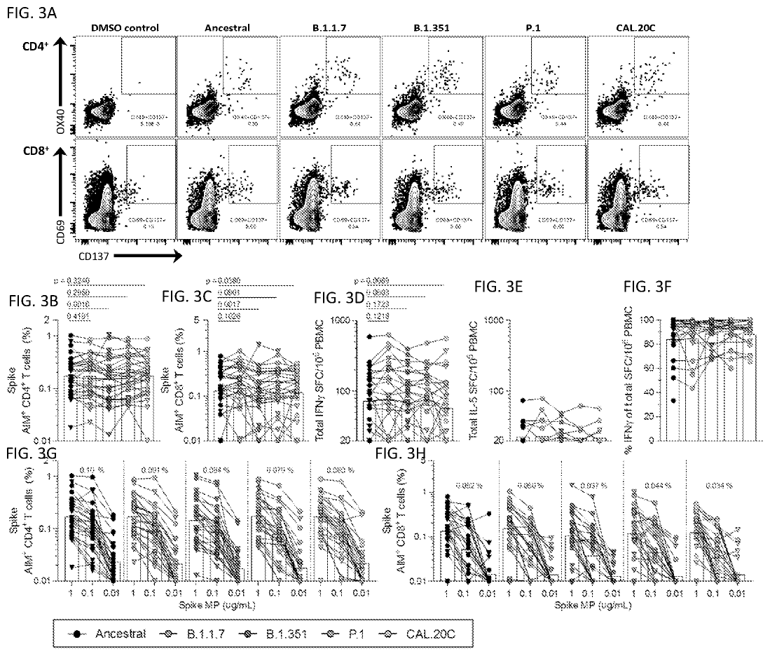

For both CD4⁺ and CD8⁺ T cell reactivity, the magnitude of responses to pools encompassing the sequences from the ancestral Wuhan genome and the VOCs ranged from a geomean of 0.15 to 0.17 for CD4⁺T cells and a geomean of 0.10 to 0.15 for CD8⁺ T cells (FIGS. 3A-C). Comparison of the variant pools to the ancestral sequence showed no significant differences for CD4⁺ and CD8⁺ T cell reactivity in the AIM assay for B.1.1.7 and P.1 (CD4: B.1.1.7 p=0.41; P.1 p=0.29; CD8: B.1.1.7 p=0.10; P.1 p=0.09 by Wilcoxon test). A 14% and 22% decrease, respectively, was observed with the B.1.351 pools for CD4⁺ and CD8⁺ T cells (B.1.351: p<0.01 for both comparisons), and a 10% decrease with the CAL.20C pool for CD8⁺ T cells (p=0.04) (FIGS. 3A-C). The FluoroSPOT assay (FIGS. 3D-F) showed IFNγ reactivity with geomeans ranging from 58 to the 74 SFC per million PBMCs (FIG. 3C). Given the number of comparisons made, and that the reductions observed were on the low side (and others showed increases), the inventors decided to put these numbers into context by comparing them to what is expected based on the observed fold-changes in repeat measurements of the same samples on different days for both AIM and FluoroSPOT assays, and asked if the observed decreases were significantly higher (non-inferiority analysis; see methods). No significant differences were observed, indicating that the magnitude of decreases for some strains was within expected technical assay variability. Minimal IL-5 responses were observed, with geomean reactivity ranging from 22 to 23 SFC/10⁶, which is slightly above the limit of detection (FIG. 3E). On a per donor basis, the IFNγ response was found to account for more than 80% of the total response, on average (range 84% to 89%), irrespective of whether the ancestral strain or VOCs were considered (FIG. 3F).

Similar to the experiments in convalescent donors, the inventors also examined T cell dose responses in vaccinated donors. As shown in FIGS. 3G-H, CD4⁺ and CD8⁺ T cell dose responses to the ancestral and VOC S pools were similar.

Phenotypes of CD4⁺ and CD8⁺ T cells. Next, the inventors assessed the quality of AIM⁺ T cell responses by phenotyping S-specific CD4⁺ and CD8⁺ AIM⁺ T cell responses. Consistent with previous observations^{39,48} AIM⁺ T cell responses in COVID-19 convalescent donors above the threshold of positivity (CD4>0.016% and CD8>0.16%, as described in the methods), irrespective of the variant analyzed, had a memory phenotype, preferentially enriched for central and effector memory phenotypes for CD4⁺ T cells (CD4⁺Tcm CD45RA⁺CCR7⁻ and CD4⁺Tem CD45RA⁻CCR7⁻) and terminal effector memory phenotypes for CD8⁺ T cells (CD8⁺Temra CD45RA⁺CCR7⁻). Likewise, AIM+ T cell responses in COVID-19 vaccinees had a memory phenotype regardless of the origin of the SARS-CoV-2 sequences analyzed, with preferential enrichment for Tcm and Tem for CD4 and Tem and Temra for CD8. This provides additional evidence that donors primed by the ancestral strain S protein, by either infection or vaccination, mount a memory T cell response able to cross-recognize the SARS-CoV-2 VOCs.

Conservation analysis of sets of defined CD4⁺ and CD8⁺ T cell epitopes. An analysis of experimentally defined CD4 and CD8 epitopes is presented in FIGS. 4A to 4M and examines both the number of epitopes and the experimentally-determined magnitude of responses associated with the epitopes. Specifically, the inventors recently reported a comprehensive study of epitopes recognized in convalescent subjects, leading to the identification of 280 different CD4⁺ T cell epitopes⁵¹. Here, the inventors analyzed how many of those epitopes would be impacted by mutations in the different variants. As shown in FIG. 4A, it was found that 89.6%, 90%, 94.3% and 97.1% (average 93%) of the CD4⁺ T cell epitopes are conserved in the B.1.1.7, B1.351, P.1 and CAL.20C variants. A similar pattern is observed when the magnitude of T cell responses associated with the various epitopes is considered, rather than the simple number of epitopes. Fully conserved CD4⁺ T cell epitopes account for 84.4%, 88.1%, 95.7% and 97.8% (average 91.5%) of the recognition of the B.1.1.7, B.1.351, P.1 and CAL.20C variants, respectively, based on the ancestral sequence (FIG. 4B).

That same study also reported 523 CD8⁺ T cell epitopes⁵¹. Performing a similar analysis, 508 (97.1%) of these 523 CD8⁺ T cell epitopes are totally conserved within the B.1.1.7 variant, 509 (97.3%) within the B.1.351 and P.lvariants, and 512 (97.9%) within the CAL.20C variant (FIG. 4C). Similarly, in terms of magnitude of the CD8⁺ T cell responses associated with the various epitopes, fully conserved CD8⁺T cell epitopes can be inferred to account for 98.3%, 98.4%, 97.9% and 97.8% of the recognition of the B.1.1.7, B.1.351, P.1 and CAL.20C variants, respectively, based on the ancestral sequence (FIG. 4D, average of 98.1%).

Finally, the inventors analyzed the degree of CD4⁺ and CD8⁺ T cell epitope conservation with an analysis restricted to epitopes in the Spike antigen. The number of S-derived epitopes conserved at 100% sequence identity was, on average, 84.5% for the CD4⁺ T cell epitopes (FIG. 4E), and 95.3% for the CD8⁺T cell epitopes (FIG. 4G). Similarly, in terms of magnitude of CD4⁺ T cell responses to S epitopes, fully conserved CD4⁺ T cell epitopes account for 95.5%, 75.3%, 89.8% and 98.3% of the recognition of B.1.1.7, B.1.351, P.1 and CAL.20C variants, respectively, with an average of 89.7% (FIG. 4F). For CD8⁺ T cell responses, fully conserved epitopes can be inferred to account for 95.2%, 97.6%, 95.4% and 97.3% of the recognition of B.1.1.7, B.1.351, P.1 and CAL.20C variants respectively, with an average of 96.4% (FIG. 4H).

While HLA restriction of the class II epitopes⁵¹ could not be unequivocally assigned, the restriction of the class I epitopes is based on HLA allele specific predictions and testing in HLA matched donors. Accordingly, the inventors analyzed the predicted binding affinity for each epitope and matching variant for the corresponding putative HLA class I restriction element. The predicted binding affinity for each ancestral epitope/matching variant for B.1.1.7, B.1.351, P.1 and CAL.20C variants is shown in FIGS. 4I-L and summarized in FIG. 4M. Predicted binding capacity was determined using the NetMHCpan BA 4.1 tool provided by the IEDB's analysis resource.^{54,55}. In the case of the B.1.1.7, B.1.351, P.1 and CAL.20C variants, the number of mutations associated with binding capacity decreases (conservatively defined as a 2-fold reduction) was 4 out of 15, 3 out of 14, 3 out of 14 and 6 out of 11, respectively.

In conclusion, the analyses show that the vast majority of CD4⁺ and CD8⁺ T cell epitopes are unaffected by mutations found in all the different variants. The corresponding mutations are also predicted to have minor effects on the total T cell response, thus providing a molecular basis for the overall impact on T cell reactivity by COVID-19 convalescent subjects and recipients of COVID-19 mRNA vaccines.

The present disclosure addresses a key knowledge gap pertaining to the potential of emergent SARS-CoV-2 variants to evade the overall recognition by human immune responses. The inventors focused on T cell responses elicited by either natural infection or vaccination with the Pfizer/BioNTech and Moderna COVID-19 mRNA vaccines. The inventors found similar total CD4⁺ or CD8⁺ T cell reactivity against the four variants investigated herein, B.1.1.7, B.1.351, P.1 and CAL.20C lineages found initially in the UK, South Africa, Brazil, and California, respectively. To assess the overall total T cell functionalities, the comparison between the original Wuhan isolate and the variants was performed utilizing different T cell methodologies, such as the AIM assay (quantifying T cells with a range of functionalities), and the FluoroSPOT assay (quantifying cells with specific cytokine-secreting activity). The inventors also tested whether any of the variant sequences might be associated with an altered cytokine polarization.

The data herein provide positive news in light of justified concern over the impact of SARS-CoV-2 VOCs on efforts to control and eliminate the present pandemic. Multiple VOCs are associated with increased transmissibility, and several have been associated with decreased susceptibility to neutralizing antibodies from infected or vaccinated individuals. In contrast, the data presented here show that the total T cell responses are not majorly disrupted by the VOCs. While it is not anticipated that circulating memory T cells would be effective in preventing SARS-CoV-2 infection, they can contribute in reducing COVID-19 severity^{33,56}. Several lines of evidence support this notion, such as observations that early SARS-CoV-2 T cell responses are associated with milder COVID-19^{34,35}. Thus, the T cell response may contribute to limiting COVID-19 severity induced by VOCs that partially or largely escape neutralizing antibodies. This is consistent with T cell mediated immunity observed in humans against a different respiratory pathogen, influenza, for which heterologous immunity against diverse influenza strains is associated with memory T cells to conserved epitopes⁵⁷⁻⁵⁹.

These data also provide insights on the predicted impact of the mutations associated with the variants analyzed on T cell responses in the context of the T cell epitopes recognized. Prior reports have identified a large number of T cell epitopes recognized throughout the SARS-CoV-2 proteome, including Spike^{40,53,60-63}. The inventors furthered this point by an analysis of the Tarke et al. data set, showing that 93% of CD4⁺ and 97% of CD8⁺ T cell epitopes in SARS-CoV-2 are completely conserved in the variants. Further, the inventors found that even in the epitopes affected by single mutations, no negative affect on the predicted HLA binding capacity in the majority of cases is expected. By way of explanation and in no way a limitation of the present disclosure, the inventors hypothesize that single amino acid substitutions or deletions across large peptidomes do not majorly impact a polyclonal memory T cell response. The apparent higher conservation of CD8⁺ T cell epitopes is to be expected based on the shorter length of HLA class I binding peptides (usually 9-10 amino acids) as compared to their class II counterparts (13-17). This effect is counterbalanced by CD8⁺T cells being generally less tolerant of amino substitutions as compared to CD4⁺ T cells^{47,64}. Marginal IL-5 production was detected in the conditions tested. This is relevant, since it was reported that single amino acid replacements in an epitope sequence can lead to a change in the cytokines produced^{65,66}, and a Th2-like response pattern was initially hypothesized to be linked to adverse outcomes in SARS-specific responses⁶⁷. Overall, the inventors observed that VOC mutations do not majorly disrupt the total CD4⁺ and CD8⁺ T cell responses.

VOC mutations could be reflective of adaptation in terms of optimizing replication or binding to ACE2, but also reflective of adaptation to escape immune recognition by antibodies^{13,14,22,68,69}. Indeed, higher viral binding to a cellular receptor can be a mechanism of compensatory viral evolution in the presence of neutralizing antibodies⁷⁰. In that respect, while mutations to escape antibody binding have been well documented for influenza⁷¹⁻⁷³ and SARS-CoV-2, immune escape at the level of T cell responses in human populations has not been reported for acute respiratory infections. Because of HLA polymorphism, the epitope repertoire recognized is likely to be substantially different from one individual to the next, greatly decreasing the likelihood of immune escape by an acute virus. Thus, an advantage conferred to the virus by a mutation in one person would not be linked to an immune response escape advantage in a non-HLA matched individual. For SARS-CoV-2, this property of T cell recognition is further enhanced by the fact that the T cell responses against SARS-CoV-2 are highly multi-antigenic and multi-specific, with tens of different epitopes recognized by CD4⁺ and CD8⁺ T cells in a given individual^{51,52,60,62}. Nevertheless, these data do not rule out that certain individuals could be strongly affected by the mutations in specific VOCs.

These results can be used to engineer coronavirus vaccines with broader protective immunity against VOCs. For example, current vaccines can be updates with the T cell epitopes disclosed herein to target a variant Spike, given how highly successful several COVID-19 vaccines have proven to be against the parental SARS-CoV-2 strain. These results show that a parallel alternative approach involving inclusion of additional antigens and T cell epitopes can also be used in which T cells epitopes are selected based on a low mutational propensity⁷⁴, to ensure that neutralizing antibodies are complemented with T cell responses to minimize COVID-19 morbidity and mortality.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

The term “multimerization domain” as used herein can be any type of molecule that is directly or indirectly associated with one or more MHC/peptide monomers. A multimerization domain is a molecule, a complex of molecules, or a solid support, to which one or more MHC and/or MHC/peptide monomers can be attached. A multimerization domain can consist of one or more carriers and/or one or more scaffolds and may also contain one or more linkers connecting carrier to scaffold, carrier to carrier, and/or scaffold to scaffold. The multimerization domain may also contain one or more linkers that can be used for attachment of MHC/peptide monomers and/or other molecules to the multimerization domain. In this disclosure, a multimerization domain will in one embodiment refer to a functionalized polymer (e.g., dextran) that is capable of reacting with MHC/peptide monomers, thus covalently attaching the MHC/peptide monomer to the multimerization domain, or that is capable of reacting with scaffold molecules (e.g., streptavidin), thus covalently attaching streptavidin to the multimerization domain; the streptavidin then may bind MHC/peptide monomers. Multimerization domains include IgG, streptavidin, avidin, streptactin, micelles, cells, polymers, dextran, polysaccharides, beads and other types of solid support, and small organic molecules carrying reactive groups or carrying chemical motifs that can bind MHC/peptide monomers and other molecules, such as identified in detail herein elsewhere.

Non-limiting examples of suitable multimerization domain(s) are polysaccharides including dextran molecules, carboxy methyl dextran, dextran polyaldehyde, carboxymethyl dextran lactone, and cyclodextrins, pullulans, schizophyllan, scleroglucan, xanthan, gellan, O-ethylamino guaran, chitins and chitosans including 6-O-carboxymethyl chitin and N-carboxymethyl chitosan, derivatized cellolosics including carboxymethyl cellulose, carboxymethyl hydroxyethyl cellulose, hydroxy-ethyl cellulose, 6-amino-6-deoxy cellulose and O-ethyl-amine cellulose, hydroxylated starch, hydroxypropyl starch, hydroxyethyl starch, carrageenans, alginates, and agarose, synthetic polysaccharides including ficoll and carboxy-methylated ficoll, vinyl polymers including poly (acrylic acid), poly (acryl amides), poly (acrylic esters), poly (2-hydroxy ethyl methacrylate), poly (methyl methacrylate), poly (maleic acid), poly (maleic anhydride), poly (acrylamide), poly (ethyl-co-vinyl acetate), poly (methacrylic acid), poly (vinyl-alcohol), poly (vinyl alcohol-co-vinyl chloroacetate), aminated poly (vinyl alcohol), and co block polymers thereof, poly ethylene glycol (PEG) or polypropylene glycol or poly (ethylene oxide-co-propylene oxides) comprising polymer backbones including linear, comb-shaped or starburst dendrimers, poly amino acids including polylysines, polyglutamic acid, polyurethanes, poly (ethylene imines), pluriol, proteins including peptides, polypeptides, antigen binding peptides, albumins, immunoglobulins, coiled-coil helixes e.g. Fos-Jun or Fos-Jun like or coiled-coiled dimers/trimers/tetramers/pentamers, Streptavidin, Avidin, STREP-TACTIN®, T-cell receptors other protein receptors and virus-like proteins (VLP), and polynucleotides, DNA, RNA, PNA, LNA, oligonucleotides and oligonucleotide dendrimer constructs and small organic molecules including but not limited to steroids, peptides, linear or cyclic structures, aromatic structures, aliphatic structures.

The term “Dextran” as used herein is in a preferred embodiment a complex, branched polysaccharide made of many glucose molecules joined into chains of varying lengths. The straight chain consists of α1->6 glycosidic linkages between glucose molecules, while branches begin from α1->3 linkages (and in some cases, α1->2 and α1->4 linkages as well).

The term “label” is used interchangeable with labeling molecule. Label as described herein is an identifiable substance that is detectable in an assay and that can be attached to a molecule creating a labeled molecule. The behavior of the labeled molecule can then be studied. Labels may be organic or inorganic molecules or particles. Labels may be organic or inorganic molecules or particles. Examples of labels include, but are not limited to, polymers, nucleic acids, DNA, RNA, oligonucleotides, peptides, fluorescent labels, phosphorescent labels, enzyme labels, chemiluminescent labels, bioluminescent labels, haptens, antibodies, dyes, nanoparticle labels, elements, metal particles, heavy metal labels, isotope labels, radioisotopes, stable isotopes, chains of isotopes and single atoms, or combination thereof. The labelling compound may suitably be selected from fluorescent labels such as 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, Green fluorescent protein (GFP) and analogues thereof, and conjugates of R-phycoerythrin or allophycoerythrin and e.g. Cy5 or Texas Red, and inorganic fluorescent labels based on semiconductor nanocrystals (like quantum dot and Qdot™ nanocrystals), and time-resolved fluorescent labels based on lanthanides like Eu3+ and Sm3+. In one embodiment a MHC monomer or MHC multimer as defined herein comprises at least one nucleic acid label, such as a nucleotide label, for example an oligonucleotide label. Such nucleic acids labels are disclosed in WO 2015/188839 and WO 2015/185067 (which are hereby incorporated by reference).

The MHC/peptide multimer can comprise one or more labels such as only a singly label. The one or more labels can be directly attached to the MHC/peptide multimer or indirectly to the MHC/peptide multimer such as via one or more marker molecules carrying one or more labels. The one or more labels can be used for combinatorial use of labelling. The one or more labels can result in positive selection of said MHC/peptide multimer or alternatively in negative selection of said MHC/peptide multimer. The one or more labels can comprise one or more covalently attached labels and/or one or more non-covalently attached labels. The one or more labels can be covalently attached to polypeptide a of the MHC monomer, covalently attached to polypeptide b of the MHC monomer, covalently attached to the peptide and/or covalently attached to the one or more multimerization domains. Alternatively, the one or more labels can be non-covalently attached to polypeptide a of the MHC monomer, non-covalently attached to polypeptide b of the MHC monomer, non-covalently attached to the peptide and/or non-covalently attached to the one or more multimerization domains. In another embodiment the one or more labels can be covalently and/or non-covalently attached to the multimerization domain via a molecule, wherein the molecule e.g., can be selected from the group consisting of an antibody, an aptamer, a protein, a sugar residue and a nucleotide such as DNA. In a specific embodiment the one or more labels are attached to the MHC/peptide multimer via a streptavidin-biotin linkage.

In a particular embodiment the label is an oligonucleotide, such as a nucleic acid molecule comprises or consists of DNA, RNA, and/or artificial nucleotides such as PLA or LNA. In one embodiment the nucleic acid label comprises one or more of the following components: a barcode region, 5′ first primer region (forward), 3′ second primer region (reverse), random nucleotide region, connector molecule, stability-increasing components, short nucleotide linkers in between any of the above-mentioned components, adaptors for sequencing and annealing region. Preferably the nucleic acid label comprises at least a barcode region; where the barcode region comprises a sequence of consecutive nucleic acids. In one embodiment the nucleic acid label comprises or consists of DNA, RNA, artificial nucleic acids and/or Xeno nucleic acid (XNA). In one embodiment at least two different labels are attached to a MHC monomer or a MHC multimer, such as at least two different labels such as one fluorescent label and one nucleic acid label. The MHC/peptide multimer can comprise one or more fluorescent labels selected from the group of fluorescein isothiocyanate, rhodamine, phycoerythrin, phycocyanin, allophycocyanin, o-phthaldehyde, fluorescamine, 2-4′-maleimidylanilino)naphthalene-6-sulfonic acid sodium salt, 5-((((2-iodoacetyl)amino)ethyl)amino), naphthalene-1-sulfonic acid, Pyrene-1-butanoic acid, AlexaFluor 350 (7-amino-6-sulfonic acid-4-methyl coumarin-3-acetic acid, AMCA (7-amino-4-methyl coumarin-3-acetic acid), 7-hydroxy-4-methyl coumarin-3-acetic acid, Marina Blue (6,8-difluoro-7-hydroxy-4-methyl coumarin-3-acetic acid), 7-dimethylamino-coumarin-4-acetic acid, Fluorescamin-N-butyl amine adduct, 7-hydroxy-coumarine-3-carboxylic acid, CascadeBlue (pyrene-trisulphonic acid acetyl azide), Cascade Yellow, Pacific Blue (6,8 difluoro-7-hydroxy coumarin-3-carboxylic acid), 7-diethylamino-coumarin-3-carboxylic acid, N-(((4-azidobenzoyl)amino)ethyl)-4-amino-3,6-disulfo-1,8-naphthalimide, dipotassium salt), Alexa Fluor 430, 3-perylenedodecanoic acid, 8-hydroxypyrene-1,3,6-trisulfonic acid trisodium salt, 12-(N-(7-nitrobenz-2-oxa-1,3-diazol-4-yl)amino)dodecanoic acid, N,N′-dimethyl-N-(iodoacetyl)-N′-(7-nitrobenz-2-oxa-1,3-diazol-4-yl)ethylenediamine, Oregon Green 488 (difluoro carboxy fluorescein), 5-iodoacetamidofluorescein, propidium iodide-DNA adduct, Carboxy fluorescein, fluor dyes, Pacific Blue™, Pacific Orange™, Cascade Yellow™, AlexaFluor® (AF), AF®350, AF405, AF430, AF488,AF500, AF514, AF532, AF546, AF555, AF568, AF594, AF610, AF633, AF635, AF647, AF680, AF700, AF710, AF750, AF800, Quantum Dot based dyes, QDot® Nanocrystals (Invitrogen, MolecularProbs), Qdot®525, Qdot®565, Qdot®585, Qdot®605, Qdot®655, Qdot®705, Qdot®800, DyLight™ Dyes (Pierce) (DL); DL549, DL649, DL680, DL800, Fluorescein (Flu) or any derivate of that, such as FITC, Cy-Dyes, Cy2, Cy3, Cy3.5, Cy5, Cy5.5, Cy7, 7-AAD, TO-Pro-3, fluorescent Proteins, R-Phycoerythrin (RPE), Phycobili Proteins, Allophycocyani (APC), PerCp, B-Phycoerythrin, C-Phycocyanin, APC, fluorescent proteins, Green fluorescent proteins; GFP and GFP derivated mutant proteins; BFP, CFP, YFP, DsRed, DSred-2, T1, Dimer2, mRFP1, MBanana, mOrange, dTomato, tdTomato, mTangerine, mStrawberry, mCherry, Tandem dyes, RPE-Cy5, RPE-Cy5.5, RPE-Cy7, RPE-AlexaFluor® tandem conjugates; RPE-Alexa610, RPE-TxRed, Tandem dyes with APC, APC-Aleca600, APC-Alexa610, APC-Alexa750, APC-Cy5, APC-Cy5.5, multi fluorochrome assemblies, FRET-based dyes (Fluorescence resonance energy transfer), ionophors; ion chelating fluorescent props, props that change wavelength when binding a specific ion, such as Calcium, props that change intensity when binding to a specific ion, such as Calcium, Calcium dyes, Indo-1-Ca2+, Indo-2-Ca2+.

The one or more labels can in a specific embodiment be selected from the group consisting of APC, APC-Cy7, ABC-H7, APC-R700, Alexa Flours™ 488, Alexa Flours™555, Alexa Flours™647, Alexa Flours™700, AmCyan, BB151, BB700, BUV395, BUV496, BUV563, BUV615, BUV661, BUV737, BUV805, BV421, BV480, BV510, BV605, BV711, BV750, BV786, FITC, PE, PE-CF594, PE-Cy5, PE-CY5.5, PE-cy7, Pasific Blue, PERCP, pPerCp-Cy5.5, PE, R718, RY586, V450 and V500 (wherein in BV means Brilliant violet, wherein BUV means Brilliant ultra violet and PE means R-Phycoerythrin). In another embodiment the one or more labels can be selected from the group consisting of cFluor®B515, cFluor®B532, cFluor®B548, cFluor®B675, cFluor®B690, cFluor®BY575, cFluor®BY610, cFluor®BY667, cFluor®BY710, cFluor®BY750, cFluor®BY781, cFluor®B250, cFluor®R659, cFluor®R668, cFluor®R685, cFluor®R720, cFluor®R780, cFluor®R840, cFluor®v420, cFluor®v547, cFluor®v450, cFluor®v610 and cFluor®YG610. The MHC/peptide multimer can comprise one or more labels which are one or more chemiluminescent label such as one or more labels selected from the group consisting of luminol, isoluminol, theromatic acridinium ester, imidazole, acridinium salt and oxalate ester. The MHC/peptide multimer can comprise one or more labels which are one or more bioluminescent labels such as one or more labels selected from the group consisting of luciferin, luciferase and aequorin. The MHC/peptide multimer can comprise one or more labels which are one or more enzyme labels, such as one or more enzyme labels selected from the group peroxidases, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The MHC/peptide multimer can comprise one or more labels which are one or more chromophore labels. In another embodiment the MHC/peptide multimer comprises one or more labels which are one or more metal labels. In yet another embodiment the MHC/peptide multimer comprises one or more labels which are one or more radioactive labels such as one or more labels selected from the group consisting of a radionuclide, an isotope, a label comprising a rays, a label comprising β rays or a label comprising γ rays. Any of the above embodiments regarding labels can be combined in any order.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Kirby, T. (2021). New variant of SARS-CoV-2 in UK causes surge of COVID-19. The Lancet. Respiratory medicine 9, e20-e21. 10.1016/S2213-2600(21)00005-9.
2. Volz, E., Mishra, S., Chand, M., Barrett, J. C., Johnson, R., Geidelberg, L., Hinsley, W. R., Laydon, D. J., Dabrera, G., O'Toole, A., et al. (2021). Assessing transmissibility of SARS-CoV-2 lineage B.1.1.7 in England. Nature 593, 266-269. 10.1038/s41586-021-03470-x.
3. Davies, N. G., Abbott, S., Barnard, R. C., Jarvis, C. I., Kucharski, A. J., Munday, J. D., Pearson, C. A. B., Russell, T. W., Tully, D. C., Washburne, A. D., et al. (2021). Estimated transmissibility and impact of SARS-CoV-2 lineage B.1.1.7 in England. Science 372. 10.1126/science.abg3055.
4. Tegally, H., Wilkinson, E., Lessells, R. J., Giandhari, J., Pillay, S., Msomi, N., Mlisana, K., Bhiman, J. N., von Gottberg, A., Walaza, S., et al. (2021). Sixteen novel lineages of SARS-CoV-2 in South Africa. Nat Med 27, 440-446. 10.1038/s41591-021-01255-3.
5. Voloch, C. M., da Silva Francisco, R., Jr., de Almeida, L. G. P., Cardoso, C. C., Brustolini, O. J., Gerber, A. L., Guimaraes, A. P. C., Mariani, D., da Costa, R. M., Ferreira, O. C., Jr., et al. (2021). Genomic characterization of a novel SARS-CoV-2 lineage from Rio de Janeiro, Brazil. J Virol. 10.1128/JVI.00119-21.
6. Faria, N. R., Mellan, T. A., Whittaker, C., Claro, I. M., Candido, D. d. S., Mishra, S., Crispim, M. A. E., Sales, F. C. S., Hawryluk, I., McCrone, J. T., et al. (2021). Genomics and epidemiology of the P.1 SARS-CoV-2 lineage in Manaus, Brazil. Science, eabh2644. 10.1126/science.abh2644.
7. Zhang, W., Davis, B. D., Chen, S. S., Sincuir Martinez, J. M., Plummer, J. T., and Vail, E. (2021). Emergence of a Novel SARS-CoV-2 Variant in Southern California. JAMA 325, 1324-1326. 10.1001/jama.2021.1612.
8. Deng, X., Garcia-Knight, M. A., Khalid, M. M., Servellita, V., Wang, C., Morris, M. K., Sotomayor-González, A., Glasner, D. R., Reyes, K. R., Gliwa, A. S., et al. (2021). Transmission, infectivity, and antibody neutralization of an emerging SARS-CoV-2 variant in California carrying a L452R spike protein mutation. medRxiv, 2021.2003.2007.21252647. 10.1101/2021.03.07.21252647.
9. Rambaut, A., Loman, N., Pybus, O., Barclay, W., Barrett, J., Carabelli, A., Connor, T., Peacock, T., Robertson, D. L., and E, V. (2020). Preliminary genomic characterisation of an emergent SARS-CoV-2 lineage in the UK defined by a novel set of spike mutations. https://virological.org/t/preliminary-genomic-characterisation-of-an-emergent-sars-cov-2-lineage-in-the-uk-defined-by-a-novel-set-of-spike-mutations/563.
10. Washington, N. L., Gangavarapu, K., Zeller, M., Bolze, A., Cirulli, E. T., Barrett, K. M. S., Larsen, B. B., Anderson, C., White, S., Cassens, T., et al. (2021). Genomic epidemiology identifies emergence and rapid transmission of SARS-CoV-2 B.1.1.7 in the United States. medRxiv, 2021.2002.2006.21251159. 10.1101/2021.02.06.21251159.
11. Greaney, A. J, Loes, A. N., Crawford, K. H. D., Starr, T. N., Malone, K. D., Chu, H. Y., and Bloom, J. D. (2021). Comprehensive mapping of mutations in the SARS-CoV-2 receptor-binding domain that affect recognition by polyclonal human plasma antibodies. Cell host & microbe. 10.1016/j.chom.2021.02.003.
12. Starr, T. N., Greaney, A. J, Addetia, A., Hannon, W. W., Choudhary, M. C., Dingens, A. S., Li, J. Z., and Bloom, J. D. (2021). Prospective mapping of viral mutations that escape antibodies used to treat COVID-19. Science 371, 850-854. 10.1126/science.abf9302.
13. Zahradnik, J., Marciano, S., Shemesh, M., Zoler, E., Chiaravalli, J., Meyer, B., Dym, O., Elad, N., and Schreiber, G. (2021). SARS-CoV-2 RBD <em>in vitro</em>evolution follows contagious mutation spread, yet generates an able infection inhibitor. bioRxiv, 2021.2001.2006.425392. 10.1101/2021.01.06.425392.
14. Wang, P., Nair, M. S., Liu, L., Iketani, S., Luo, Y., Guo, Y., Wang, M., Yu, J., Zhang, B., Kwong, P. D., et al. (2021). Antibody resistance of SARS-CoV-2 variants B.1.351 and B.1.1.7. Nature. 10.1038/s41586-021-03398-2.
15. Skelly, D. T., Harding, A. C., Gilbert-Jaramillo, J., Knight, M. L., Longet, S., Brown, A., Adele, S., Adland, E., Brown, H., Medawar Laboratory Team, et al. (2020). Vaccine-induced immunity provides more robust heterotypic immunity than natural infection to emerging SARS-CoV-2 variants of concern. Research square. 10.21203/rs.3.rs-226857/v1.
16. Wu, K., Werner, A. P., Koch, M., Choi, A., Narayanan, E., Stewart-Jones, G. B. E., Colpitts, T., Bennett, H., Boyoglu-Barnum, S., Shi, W., et al. (2021). Serum Neutralizing Activity Elicited by mRNA-1273 Vaccine—Preliminary Report. N Engl J Med. 10.1056/NEJMc2102179.
17. Muik, A., Wallisch, A. K., Sanger, B., Swanson, K. A., Muhl, J., Chen, W., Cai, H., Maurus, D., Sarkar, R., Tureci, O., et al. (2021). Neutralization of SARS-CoV-2 lineage B.1.1.7 pseudovirus by BNT162b2 vaccine-elicited human sera. Science 371, 1152-1153. 10.1126/science.abg6105.
18. Supasa, P., Zhou, D., Dejnirattisai, W., Liu, C., Mentzer, A. J., Ginn, H. M., Zhao, Y., Duyvesteyn, H. M. E., Nutalai, R., Tuekprakhon, A., et al. (2021). Reduced neutralization of SARS-CoV-2 B.1.1.7 variant by convalescent and vaccine sera. Cell 184, 2201-2211 e2207. 10.1016/j.cell.2021.02.033.
19. Edara, V. V., Norwood, C., Floyd, K., Lai, L., Davis-Gardner, M E., Hudson, W. H., Mantus, G., Nyhoff, L. E., Adelman, M. W., Fineman, R., et al. (2021). Infection- and vaccine-induced antibody binding and neutralization of the B.1.351 SARS-CoV-2 variant. Cell host & microbe. https://doi.org/10.1016/j.chom.2021.03.009.
20. Shen, X., Tang, H., McDanal, C., Wagh, K., Fischer, W., Theiler, J., Yoon, H., Li, D., Haynes, B. F., Sanders, K. O., et al. (2021). SARS-CoV-2 variant B.1.1.7 is susceptible to neutralizing antibodies elicited by ancestral spike vaccines. Cell host & microbe 29, 529-539 e523. 10.1016/j.chom.2021.03.002.
21. Stamatatos, L., Czartoski, J., Wan, Y. H., Homad, L. J., Rubin, V., Glantz, H., Neradilek, M., Seydoux, E., Jennewein, M. F., MacCamy, A. J., et al. (2021). mRNA vaccination boosts cross-variant neutralizing antibodies elicited by SARS-CoV-2 infection. Science. 10.1126/science.abg9175.
22. Wang, Z., Schmidt, F., Weisblum, Y., Muecksch, F., Barnes, C. O., Finkin, S., Schaefer-Babajew, D., Cipolla, M., Gaebler, C., Lieberman, J. A., et al. (2021). mRNA vaccine-elicited antibodies to SARS-CoV-2 and circulating variants. Nature. 10.1038/s41586-021-03324-6.
23. Wibmer, C. K., Ayres, F., Hermanus, T., Madzivhandila, M., Kgagudi, P., Oosthuysen, B., Lambson, B. E., de Oliveira, T., Vermeulen, M., van der Berg, K., et al. (2021). SARS-CoV-2 501Y. V2 escapes neutralization by South African COVID-19 donor plasma. Nat Med. 10.1038/s41591-021-01285-x.
24. Emary, K. R. W., Golubchik, T., Aley, P. K., Ariani, C. V., Angus, B., Bibi, S., Blane, B., Bonsall, D., Cicconi, P., Charlton, S., et al. (2021). Efficacy of ChAd0x1 nCoV-19 (AZD1222) vaccine against SARS-CoV-2 variant of concern 202012/01 (B.1.1.7): an exploratory analysis of a randomised controlled trial. Lancet 397, 1351-1362. 10.1016/S0140-6736(21)00628-0.
25. Cele, S., Gazy, I., Jackson, L., Hwa, S. H., Tegally, H., Lustig, G., Giandhari, J., Pillay, S., Wilkinson, E., Naidoo, Y., et al. (2021). Escape of SARS-CoV-2 501Y. V2 from neutralization by convalescent plasma. Nature. 10.1038/s41586-021-03471-w.
26. Voysey, M., Clemens, SAC., Madhi, S. A., Weckx, L. Y., Folegatti, P. M., Aley, P. K., Angus, B., Baillie, V. L., Barnabas, S. L., Bhorat, Q. E., et al. (2021). Safety and efficacy of the ChAd0x1 nCoV-19 vaccine (AZD1222) against SARS-CoV-2: an interim analysis of four randomised controlled trials in Brazil, South Africa, and the UK. Lancet 397, 99-111. 10.1016/S0140-6736(20)32661-1.
27. Hall, V. J., Foulkes, S., Saei, A., Andrews, N., Oguti, B., Charlett, A., Wellington, E., Stowe, J., Gillson, N., Atti, A., et al. (2021). Effectiveness of BNT162b2 mRNA Vaccine Against Infection and COVID-19 Vaccine Coverage in Healthcare Workers in England, Multicentre Prospective Cohort Study (the SIREN Study). http://dx.doi.org/10.2139/ssm.3790399. Ssrn. http://dx.doi.org/10.2139/ssrn.3790399.
28. Amit, S., Regev-Yochay, G., Afek, A., Kreiss, Y., and Leshem, E. (2021). Early rate reductions of SARS-CoV-2 infection and COVID-19 in BNT162b2 vaccine recipients. Lancet 397, 875-877. 10.1016/S0140-6736(21)00448-7.
29. Dagan, N., Barda, N., Kepten, E., Miron, O., Perchik, S., Katz, M. A., Hernan, M. A., Lipsitch, M., Reis, B., and Balicer, R. D. (2021). BNT162b2 mRNA Covid-19 Vaccine in a Nationwide Mass Vaccination Setting. N Engl J Med 384, 1412-1423. 10.1056/NEJMoa2101765.
30. Novavax Inc. (2021). Novavax COVID-19 Vaccine Demonstrates 89.3% Efficacy in UK Phase 3 Trial. https://ir.novavax.com/news-releases/news-release-details/novavax-covid-19-vaccine-demonstrates-893-efficacy-uk-phase-3.
31. FDA (2021). Vaccines and Related Biological Products Advisory Committee Feb. 26, 2021 Meeting Briefing Document—Sponsor. https://www.fda.gov/media/146219/download
32. FDA (2021). Vaccines and Related Biological Products Advisory Committee Feb. 26, 2021 Meeting Briefing Document Addendum—Sponsor fda.gov/media/146218/download.
33. Sette, A., and Crotty, S. (2021). Adaptive immunity to SARS-CoV-2 and COVID-19. Cell 184, 861-880. 10.1016/j.cell.2021.01.007.
34. Rydyznski Moderbacher, C., Ramirez, S. I., Dan, J. M., Grifoni, A., Hastie, K. M., Weiskopf, D., Belanger, S., Abbott, R. K., Kim, C., Choi, J., et al. (2020). Antigen-Specific Adaptive Immunity to SARS-CoV-2 in Acute COVID-19 and Associations with Age and Disease Severity. Cell 183, 996-1012 e1019. 10.1016/j.cell.2020.09.038.
35. Tan, A. T., Linster, M., Tan, C. W., Le Bert, N., Chia, W. N., Kunasegaran, K., Zhuang, Y., Tham, C. Y. L., Chia, A., Smith, G. J. D., et al. (2021). Early induction of functional SARS-CoV-2-specific T cells associates with rapid viral clearance and mild disease in COVID-19 patients. Cell reports, 108728. 10.1016/j.celrep.2021.108728.
36. Munoz-Fontela, C., Dowling, W. E., Funnell, S. G. P., Gsell, P. S., Riveros-Balta, A. X., Albrecht, R. A., Andersen, H., Baric, R. S., Carroll, M. W., Cavaleri, M., et al. (2020). Animal models for COVID-19. Nature 586, 509-515. 10.1038/s41586-020-2787-6.
37. Soresina, A., Moratto, D., Chiarini, M., Paolillo, C., Baresi, G., Foca, E., Bezzi, M., Baronio, B., Giacomelli, M., and Badolato, R. (2020). Two X-linked agammaglobulinemia patients develop pneumonia as COVID-19 manifestation but recover. Pediatric allergy and immunology. official publication of the European Society of Pediatric Allergy and Immunology 31, 565-569. 10.1111/pai.13263.
38. Baker, D., Roberts, C. A. K., Pryce, G., Kang, A. S., Marta, M., Reyes, S., Schmierer, K., Giovannoni, G., and Amor, S. (2020). COVID-19 vaccine-readiness for anti-CD20-depleting therapy in autoimmune diseases. Clin Exp Immunol 202, 149-161. 10.1111/cei.13495.
39. Dan, J. M., Mateus, J., Kato, Y., Hastie, K. M., Yu, E. D., Faliti, C. E., Grifoni, A., Ramirez, S. I., Haupt, S., Frazier, A., et al. (2021). Immunological memory to SARS-CoV-2 assessed for up to 8 months after infection. Science 371. 10.1126/science.abf4063.
40. Peng, Y., Mentzer, A. J., Liu, G., Yao, X., Yin, Z., Dong, D., Dejnirattisai, W., Rostron, T., Supasa, P., Liu, C., et al. (2020). Broad and strong memory CD4(+) and CD8(+) T cells induced by SARS-CoV-2 in UK convalescent individuals following COVID-19. Nat Immunol 21, 1336-1345. 10.1038/s41590-020-0782-6.
41. Breton, G., Mendoza, P., Hagglof, T., Oliveira, T. Y., Schaefer-Babajew, D., Gaebler, C., Turroja, M., Hurley, A., Caskey, M., and Nussenzweig, M. C. (2021). Persistent cellular immunity to SARS-CoV-2 infection. J Exp Med 218. 10.1084/jem.20202515.
42. Dowd, S. M., Zalta, A. K., Burgess, H. J., Adkins, E. C., Valdespino-Hayden, Z., and Pollack, M. H. (2020). Double-blind randomized controlled study of the efficacy, safety and tolerability of eszopiclone vs placebo for the treatment of patients with post-traumatic stress disorder and insomnia. World journal of psychiatry 10, 21-28. 10.5498/wjp.v10.i3.21.
43. Sadoff, J., Le Gars, M., Shukarev, G., Heerwegh, D., Truyers, C., de Groot, A. M., Stoop, J., Tete, S., Van Damme, W., Leroux-Roels, I., et al. (2021). Interim Results of a Phase 1-2a Trial of Ad26.COV2.S Covid-19 Vaccine. N Engl J Med. 10.1056/NEJMoa2034201.
44. Baden, L. R., El Sahly, H. M., Essink, B., Kotloff, K., Frey, S., Novak, R., Diemert, D., Spector, S. A., Rouphael, N., Creech, C. B., et al. (2021). Efficacy and Safety of the mRNA-1273 SARS-CoV-2 Vaccine. N Engl J Med 384, 403-416. 10.1056/NEJMoa2035389.
45. Keech, C., Albert, G., Cho, I., Robertson, A., Reed, P., Neal, S., Plested, J. S., Zhu, M., Cloney-Clark, S., Zhou, H., et al. (2020). Phase 1-2 Trial of a SARS-CoV-2 Recombinant Spike Protein Nanoparticle Vaccine. N Engl J Med 383, 2320-2332. 10.1056/NEJMoa2026920.
46. Redd, A. D., Nardin, A., Kared, H., Bloch, E. M., Pekosz, A., Laeyendecker, O., Abel, B., Fehlings, M., Quinn, T. C., and Tobian, A. A. R. (2021). CD8⁺ T cell responses in COVID-19 convalescent individuals target conserved epitopes from multiple prominent SARS-CoV-2 circulating variants. Open forum infectious diseases. 10.1093/ofid/ofab143.
47. Grifoni, A., Weiskopf, D., Ramirez, S. I., Mateus, J., Dan, J. M., Moderbacher, C. R., Rawlings, S. A., Sutherland, A., Premkumar, L., Jadi, R. S., et al. (2020). Targets of T Cell Responses to SARS-CoV-2 Coronavirus in Humans with COVID-19 Disease and Unexposed Individuals. Cell 181, 1489-1501 e1415. 10.1016/j.cell.2020.05.015.
48. Mateus, J., Grifoni, A., Tarke, A., Sidney, J., Ramirez, S. I., Dan, J. M., Burger, Z. C., Rawlings, S. A., Smith, D. M., Phillips, E., et al. (2020). Selective and cross-reactive SARS-CoV-2 T cell epitopes in unexposed humans. Science 370, 89-94. 10.1126/science.abd3871.
49. Zuo, J., Dowell, A. C., Pearce, H., Verma, K., Long, H. M., Begum, J., Aiano, F., Amin-Chowdhury, Z., Hallis, B., Stapley, L., et al. (2021). Robust SARS-CoV-2-specific T cell immunity is maintained at 6 months following primary infection. Nat Immunol. 10.1038/s41590-021-00902-8.
50. Le Bert, N., Clapham, H E., Tan, A. T., Chia, W. N., Tham, C. Y. L., Lim, J. M., Kunasegaran, K., Tan, L. W. L., Dutertre, C. A., Shankar, N., et al. (2021). Highly functional virus-specific cellular immune response in asymptomatic SARS-CoV-2 infection. J Exp Med 218. 10.1084/jem.20202617.
51. Tarke, A., Sidney, J., Kidd, C. K., Dan, J. M., Ramirez, S. I., Yu, E. D., Mateus, J., da Silva Antunes, R., Moore, E., Rubiro, P., et al. (2021). Comprehensive analysis of T cell immunodominance and immunoprevalence of SARS-CoV-2 epitopes in COVID-19 cases. Cell reports. Medicine 2, 100204. 10.1016/j.xcrm.2021.100204.
52. Braun, J., Loyal, L., Frentsch, M., Wendisch, D., Georg, P., Kurth, F., Hippenstiel, S., Dingeldey, M., Kruse, B., Fauchere, F., et al. (2020). SARS-CoV-2-reactive T cells in healthy donors and patients with COVID-19. Nature 587, 270-274. 10.1038/s41586-020-2598-9.
53. Le Bert, N., Tan, A. T., Kunasegaran, K., Tham, C. Y. L., Hafezi, M., Chia, A., Chng, M. H. Y., Lin, M., Tan, N., Linster, M., et al. (2020). SARS-CoV-2-specific T cell immunity in cases of COVID-19 and SARS, and uninfected controls. Nature 584, 457-462. 10.1038/s41586-020-2550-z.
54. Dhanda, S. K., Mahajan, S., Paul, S., Yan, Z., Kim, H., Jespersen, M. C., Jurtz, V., Andreatta, M., Greenbaum, J. A., Marcatili, P., et al. (2019). IEDB-AR: immune epitope database-analysis resource in 2019. Nucleic Acids Res 47, W502-W506. 10.1093/nar/gkz452.
55. Reynisson, B., Alvarez, B., Paul, S., Peters, B., and Nielsen, M. (2020). NetMHCpan-4.1 and NetMHCIIpan-4.0: improved predictions of MHC antigen presentation by concurrent motif deconvolution and integration of MS MHC eluted ligand data. Nucleic Acids Res 48, W449-W454. 10.1093/nar/gkaa379.
56. Lipsitch, M., Grad, Y. H., Sette, A., and Crotty, S. (2020). Cross-reactive memory T cells and herd immunity to SARS-CoV-2. Nat Rev Immunol 20, 709-713. 10.1038/s41577-020-00460-4.
57. Greenbaum, J. A., Kotturi, M. F., Kim, Y., Oseroff, C., Vaughan, K., Salimi, N., Vita, R., Ponomarenko, J., Scheuermann, R. H., Sette, A., and Peters, B. (2009). Pre-existing immunity against swine-origin H1N1 influenza viruses in the general human population. Proc Natl Acad Sci USA 106, 20365-20370. 10.1073/pnas.0911580106.
58. Sridhar, S., Begom, S., Bermingham, A., Hoschler, K., Adamson, W., Carman, W., Bean, T., Barclay, W., Deeks, J. J., and Lalvani, A. (2013). Cellular immune correlates of protection against symptomatic pandemic influenza. Nat Med 19, 1305-1312. 10.1038/nm.3350.
59. Wilkinson, T. M., Li, C. K., Chui, C. S., Huang, A. K., Perkins, M., Liebner, J. C., Lambkin-Williams, R., Gilbert, A., Oxford, J., Nicholas, B., et al. (2012). Preexisting influenza-specific CD4⁺ T cells correlate with disease protection against influenza challenge in humans. Nat Med 18, 274-280. 10.1038/nm.2612.
60. Ferretti, A. P., Kula, T., Wang, Y., Nguyen, D. M. V., Weinheimer, A., Dunlap, G. S., Xu, Q., Nabilsi, N., Perullo, C. R., Cristofaro, A. W., et al. (2020). Unbiased Screens Show CD8(+) T Cells of COVID-19 Patients Recognize Shared Epitopes in SARS-CoV-2 that Largely Reside outside the Spike Protein. Immunity 53, 1095-1107 e1093. 10.1016/j.immuni.2020.10.006.
61. Snyder, T. M., Gittelman, R. M., Klinger, M., May, D. H., Osborne, E. J., Taniguchi, R., Zahid, H. J., Kaplan, I. M., Dines, J. N., Noakes, M. N., et al. (2020). Magnitude and Dynamics of the T-Cell Response to SARS-CoV-2 Infection at Both Individual and Population Levels. medRxiv. 10.1101/2020.07.31.20165647.
62. Nelde, A., Bilich, T., Heitmann, J. S., Maringer, Y., Salih, H. R., Roerden, M., Lubke, M., Bauer, J., Rieth, J., Wacker, M., et al. (2021). SARS-CoV-2-derived peptides define heterologous and COVID-19-induced T cell recognition. Nat Immunol 22, 74-85. 10.1038/s41590-020-00808-x.
63. Keller, M. D., Harris, K. M., Jensen-Wachspress, M. A., Kankate, V. V., Lang, H., Lazarski, C. A., Durkee-Shock, J., Lee, P. H., Chaudhry, K., Webber, K., et al. (2020). SARS-CoV-2-specific T cells are rapidly expanded for therapeutic use and target conserved regions of the membrane protein. Blood 136, 2905-2917. 10.1182/blood.2020008488.
64. Weiskopf, D., Angelo, M. A., Sidney, J., Peters, B., Shresta, S., and Sette, A. (2014). Immunodominance changes as a function of the infecting dengue virus serotype and primary versus secondary infection. J Virol 88, 11383-11394. 10.1128/JVI.01108-14.
65. Evavold, B. D., and Allen, P. M. (1991). Separation of IL-4 production from Th cell proliferation by an altered T cell receptor ligand. Science 252, 1308-1310. 10.1126/science.1833816.
66. Sloan-Lancaster, J., and Allen, P. M. (1996). Altered peptide ligand-induced partial T cell activation: molecular mechanisms and role in T cell biology. Annu Rev Immunol 14, 1-27. 10.1146/annurev.immunol.14.1.1.
67. Peeples, L. (2020). News Feature: Avoiding pitfalls in the pursuit of a COVID-19 vaccine. Proc Natl Acad Sci USA 117, 8218-8221. 10.1073/pnas.2005456117.
68. Starr, T. N., Greaney, A. J., Hilton, S. K., Ellis, D., Crawford, K. H. D., Dingens, A. S., Navarro, M. J., Bowen, J. E., Tortorici, M. A., Walls, A. C., et al. (2020). Deep Mutational Scanning of SARS-CoV-2 Receptor Binding Domain Reveals Constraints on Folding and ACE2 Binding. Cell 182, 1295-1310 e1220. 10.1016/j.cell.2020.08.012.
69. Andreano, E., Piccini, G., Licastro, D., Casalino, L., Johnson, N. V., Paciello, I., Monego, S. D., Pantano, E., Manganaro, N., Manenti, A., et al. (2020). SARS-CoV-2 escape <em>in vitro</em>from a highly neutralizing COVID-19 convalescent plasma. bioRxiv, 2020.2012.2028.424451. 10.1101/2020.12.28.424451.
70. Hensley, S E., Das, S. R., Bailey, A. L., Schmidt, L. M., Hickman, H. D., Jayaraman, A., Viswanathan, K., Raman, R., Sasisekharan, R., Bennink, J R., and Yewdell, J. W. (2009). Hemagglutinin receptor binding avidity drives influenza A virus antigenic drift. Science 326, 734-736. 10.1126/science. 1178258.
71. Doud, M. B., Lee, J. M., and Bloom, J. D. (2018). How single mutations affect viral escape from broad and narrow antibodies to H1 influenza hemagglutinin. Nature communications 9, 1386. 10.1038/s41467-018-03665-3.
72. Andrews, S. F., Huang, Y., Kaur, K., Popova, L. I., Ho, I. Y., Pauli, N. T., Henry Dunand, C. J., Taylor, W. M., Lim, S., Huang, M., et al. (2015). Immune history profoundly affects broadly protective B cell responses to influenza. Sci Transl Med 7, 316ra192. 10.1126/scitranslmed.aad0522.
73. Krammer, F., Garcia-Sastre, A., and Palese, P. (2018). Is It Possible to Develop a “Universal” Influenza Virus Vaccine? Potential Target Antigens and Critical Aspects for a Universal Influenza Vaccine. Cold Spring Harbor perspectives in biology 10. 10.1101/cshperspect.a028845.
74. Gaiha, G. D., Rossin, E. J., Urbach, J., Landeros, C., Collins, D. R., Nwonu, C., Muzhingi, I., Anahtar, M. N., Waring, O. M., Piechocka-Trocha, A., et al. (2019). Structural topology defines protective CD8(+) T cell epitopes in the HIV proteome. Science 364, 480-484. 10.1126/science.aav5095.
75. Pickett, B. E., Sadat, E. L., Zhang, Y., Noronha, J. M., Squires, R. B., Hunt, V., Liu, M., Kumar, S., Zaremba, S., Gu, Z., et al. (2012). ViPR: an open bioinformatics database and analysis resource for virology research. Nucleic Acids Res 40, D593-598. 10.1093/nar/gkr859.
76. Vita, R., Mahajan, S., Overton, J. A., Dhanda, S. K., Martini, S., Cantrell, J R., Wheeler, D. K., Sette, A., and Peters, B. (2019). The Immune Epitope Database (IEDB): 2018 update. Nucleic Acids Res 47, D339-D343. 10.1093/nar/gky1006.
77. Reiss, S., Baxter, A. E., Cirelli, K. M., Dan, J. M., Morou, A., Daigneault, A., Brassard, N., Silvestri, G., Routy, J. P., Havenar-Daughton, C., et al. (2017). Comparative analysis of activation induced marker (AIM) assays for sensitive identification of antigen-specific CD4 T cells. PLoS One 12, e0186998. 10.1371/journal.pone.0186998.
78. da Silva Antunes, R., Pallikkuth, S., Williams, E., Esther, D. Y., Mateus, J., Quiambao, L., Wang, E., Rawlings, S. A., Stadlbauer, D., Jiang, K., et al. (2021). Differential T cell reactivity to endemic coronaviruses and SARS-CoV-2 in community and health care workers. The Journal of Infectious Diseases. 10.1093/infdis/jiabl76.

1. A composition comprising:
one or more peptides or proteins, comprising, consisting of, or consisting essentially of an amino acid sequence selected from those sequences set forth in Table 2 (SEQ ID NOS: 1 to 292), Table 3 (SEQ ID NOS: 293 to 347), or both, or a subsequence, portion, homologue, variant or derivative thereof,
a fusion protein comprising one or more amino acid sequences selected from those sequences set forth in Table 2, Table 3, or both; or
a pool of 2 or more peptides comprising, consisting of, or consisting essentially of amino acid sequences selected from those sequences set forth in Table 2, Table 3, or both; or
a polynucleotide that encodes one or more peptides or proteins, comprising, consisting of, or consisting essentially of an amino acid sequence selected from those sequences set forth in Table 2, Table 3, or both, or a subsequence, portion, homologue, variant or derivative thereof.

2.-4. (canceled)

5. The composition of claim 1, wherein at least one of:
the peptide or protein comprises a coronavirus T cell epitope,
the one or more peptides or proteins comprises a coronavirus CD8⁺ or CD4⁺ T cell epitope;
the coronavirus is SARS-CoV-2 and the SARS-CoV-2 T cell epitope is not conserved in another coronavirus;
the coronavirus is SARS-CoV-2 and the SARS-CoV-2 T cell epitope is conserved in another coronavirus;
the one or more peptides or proteins has a length from about 9-15, 15-20, 20-25, 25-30, 30-40, 40-50, 50-75 or 75-100 amino acids; or
the one or more peptides or proteins elicits, stimulates, induces, promotes, increases or enhances a T cell response to a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof.

6.-11. (canceled)

12. The composition of claim 1, further comprising formulating the one or more peptides or proteins into an immunogenic formulation with an adjuvant, and optionally, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, AS04, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands.

13.-16. (canceled)

17. The composition of claim 1, wherein the composition comprise monomers or multimers of:
peptides or proteins comprising, consisting of, or consisting essentially of:
one or more amino acid sequences selected from those sequences set forth in Table 2 (SEQ ID NOS: 1 to 292), Table 3 (SEQ ID NOS: 293 to 347), or both,
concatemers, subsequences, portions, homologues, variants or derivatives thereof, a fusion protein comprising one or more amino acid sequences selected from those sequences set forth in Table 2, Table 3, or both;
a polynucleotide that encodes one or more peptides or proteins, comprising, consisting of, or consisting essentially of an amino acid sequence selected from those sequences set forth in Table 2, Table 3, or both, or a subsequence, portion, homologue, variant or derivative thereof,
one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Table 2 (SEQ ID NOS: 1 to 292), Table 3 (SEQ ID NOS: 293 to 347), or both, in a groove of the MHC monomer or multimer; or
one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Table 3, in a groove of the (MHC) monomer or multimer.

18.-36. (canceled)

37. A method for detecting the presence of: (i) a coronavirus or (ii) an immune response relevant to coronavirus infections, vaccines or therapies, including T cells responsive to one or more coronavirus peptides, comprising:
providing one or more proteins or peptides for detection of an amount or a relative amount of, and/or the activity of, and/or the state of antigen-specific T-cells;
contacting a biological sample suspected of having coronavirus-specific T-cells to one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences set forth in Table 2 (SEQ ID NOS: 1 to 292), Table 3 (SEQ ID NOS: 293 to 347), or both, or comprise a pool of 2 or more amino acid sequences set forth in Table 2, Table 3, or both.

38. The method of claim 37, wherein detecting the amount or a relative amount of, and/or activity of antigen-specific T-cells comprises one or more steps of identification or detection of the antigen-specific T-cells and measuring the amount of the antigen-specific T-cells, and optionally:
the one or more peptides or proteins comprises 2 or more amino acid sequences selected from Table 2;
the detecting the amount or a relative amount of, and/or activity of antigen-specific T-cells comprises indirect detection and/or direct detection;
wherein the method of detecting an immune response relevant to the coronavirus comprises the following steps:
providing an MHC monomer or an MHC multimer;
contacting a population T-cells to the MHC monomer or MHC multimer; and
measuring the number, activity or state of T-cells specific for the MHC monomer or MHC multimer;
the MHC monomer or MHC multimer comprises a protein or peptide of the coronavirus;
the protein or peptide comprises a CD8⁺ or CD4⁺ T cell epitope;
the T cell epitope is not conserved in another coronavirus;
the T cell epitope is conserved in another coronavirus;
wherein the protein or peptide has a length from about 9-15, 15-20, 20-25, 25-30, 30-40, 40-50, 50-75 or 75-100 amino acids.

39.-48. (canceled)

49. The method of claim 37, wherein detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay.

50.-64. (canceled)

65. The method of claim 37, further comprising detecting a coronavirus infection or exposure in a subject, the method comprising, consisting of, or consisting essentially of:
contacting a biological sample from a subject with a composition of claim 1; and
determining if the composition elicits an immune response from the contacted cells, wherein the presence of an immune response indicates that the subject has been exposed to or infected with coronavirus; or
determining if the composition elicits an immune response from the contacted cells, wherein the presence of an immune response indicates that the subject has been exposed to or infected with SARS-CoV-2.

66.-78. (canceled)

79. A kit for the detection of coronavirus or an immune response to coronavirus in a subject comprising, consisting of or consisting essentially of:
one or more T cells that specifically detect the presence of:
one or more amino acid sequences selected from those sequences set forth in Table 2 (SEQ ID NOS: 1 to 292), Table 3 (SEQ ID NOS: 293 to 347), or both, or a subsequence, portion, homologue, variant or derivative thereof, or
a fusion protein comprising one or more amino acid sequences selected from those sequences set forth in Table 2, Table 3, or both; or
a pool of 2 or more peptides selected from the amino acid sequences set forth in Table 2, Table 3, or both.

80. (canceled)

81. The kit of claim 79, wherein the kit comprises at least one of:
one or more amino acid sequences selected from those sequences set forth in Table 3, or a subsequence, portion, homologue, variant or derivative thereof,
a fusion protein comprising one or more amino acid sequences selected from those sequences set forth in Table 3; or
a pool of 2 or more peptides selected from the amino acid sequences set forth in those sequences set forth in Table 3;
the one or more peptides or proteins comprises a coronavirus CD8⁺ or CD4⁺ T cell epitope;
the coronavirus is SARS-CoV-2 and the SARS-CoV-2 T cell epitope is not conserved in another coronavirus;
the coronavirus is SARS-CoV-2 and the SARS-CoV-2 T cell epitope is conserved in another coronavirus;
the one or more peptides or proteins has a length from about 9-15, 15-20, 20-25, 25-30, 30-40, 40-50, 50-75 or 75-100 amino acids; or
the one or more peptides or proteins elicits, stimulates, induces, promotes, increases or enhances a T cell response to a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof.

82.-85. (canceled)

86. The kit of claim 79, wherein the kit includes instruction for a diagnostic method, a process, a composition, a product, a service or component part thereof for the detection of: (i) coronavirus or (ii) an immune response relevant to coronavirus infections, vaccines or therapies, including T cells responsive to coronavirus.

87. The kit of claim 79, wherein the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay;
reagents for determining a Human Leukocyte Antigen (HLA) profile of a subject, and selecting peptides that are presented by the HLA profile of the subject for detecting an immune response to coronavirus;
one or more T cells that specifically detect the presence of:
one or more amino acid sequences selected from those sequences set forth in Table (SEQ ID NOS: 293 to 347), or a subsequence, portion, homologue, variant or derivative thereof;
a fusion protein comprising one or more amino acid sequences selected from those sequences set forth in Table 3; or
a pool of 2 or more peptides selected from the amino acid sequences set forth in those sequences set forth in Table 3.

88.-98. (canceled)

99. A method of stimulating, inducing, promoting, increasing, or enhancing an immune response against a coronavirus, a SARS-CoV-2 virus, or both, in a subject, comprising:
administering a composition of claim 1, in an amount sufficient to stimulate, induce, promote, increase, or enhance an immune response against the coronavirus in the subject.

100. The method of claim 99, wherein the immune response provides the subject with protection against a coronavirus infection or pathology, or one or more physiological conditions, disorders, illnesses, diseases or symptoms caused by or associated with coronavirus infection or pathology; or
one or more SARS-CoV-2 peptides selected from the amino acid sequences set forth in Table 2 (SEQ ID NOS: 1 to 292), Table 3 (SEQ ID NOS: 293 to 347), or both, or a subsequence, portion, homologue, variant or derivative thereof.

101.-105. (canceled)

106. The method of claim 99, wherein the stimulating, inducing, promoting, increasing, or enhancing an immune response against SARS-CoV-2 in a subject, comprises:
administering to a subject an amount of a protein or peptide or a polynucleotide that expresses the protein or peptide comprising, consisting of or consisting essentially of an amino acid sequence of the SARS-CoV-2 spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two peptides selected from the amino acid sequences set forth in Table 2 (SEQ ID NOS: 1 to 292), Table 3 (SEQ ID NOS: 293 to 347), or both or a subsequence, portion, homologue, variant or derivative thereof, in an amount sufficient to prevent, stimulate, induce, promote, increase, immunize against, or enhance an immune response against SARS-CoV-2 in the subject; or
administering to a subject an amount of a protein, peptide or a polynucleotide that expresses the protein or peptide comprising, consisting of, or consisting essentially of an amino acid sequence of a coronavirus spike, nucleoprotein, membrane, replicase polyprotein lab, protein 3a, envelope small membrane protein, non-structural protein 3b, protein 7a, protein 9b, non-structural protein 6, or non-structural protein 8a protein or peptide, or a variant, homologue, derivative or subsequence thereof, wherein the protein or peptide comprises at least two amino acid sequences selected from Table 2 (SEQ ID NOS: 1 to 292), Table 3 (SEQ ID NOS: 293 to 347), or both, or a subsequence, portion, homologue, variant or derivative thereof, in an amount sufficient to treat, prevent, or immunize the subject for SARS-CoV-2 infection, wherein the protein or peptide comprises or consists of a coronavirus T cell epitope that elicits, stimulates, induces, promotes, increases, or enhances an anti-SARS-CoV-2 T cell immune response,
wherein the method reduces SARS-CoV-2 viral titer, increases or stimulates SARS-CoV-2 viral clearance, reduces or inhibits SARS-CoV-2 viral proliferation, reduces or inhibits increases in SARS-CoV-2 viral titer or SARS-CoV-2 viral proliferation, reduces the amount of a SARS-CoV-2 viral protein or the amount of a SARS-CoV-2 viral nucleic acid, or reduces or inhibits synthesis of a SARS-CoV-2 viral protein or a SARS-CoV-2 viral nucleic acid.

107.-137. (canceled)

138. A peptide or peptides that are immunoprevalent or immunodominant in a virus obtained by a method consisting of, or consisting essentially of:
obtaining an amino acid sequence of the virus;
determining one or more sets of overlapping peptides spanning one or more virus antigen using unbiased selection;
synthesizing one or more pools of virus peptides comprising the one or more sets of overlapping peptides;
combining the one or more pools of virus peptides with Class I major histocompatibility proteins (MHC), Class II MHC, or both Class I and Class II MHC to form peptide-MHC complexes;
contacting the peptide-MHC complexes with T cells from subjects exposed to the virus;
determining which pools triggered cytokine release by the T cells; and
deconvoluting from the pool of peptides that elicited cytokine release by the T cells, which peptide or peptides are immunoprevalent or immunodominant in the pool.

139. The peptide or peptides of claim 138, wherein at least one of:
the virus is a coronavirus;
the virus is SARS-CoV-2;
the immunodominant peptides are selected from 1, 2 or more peptides selected from the amino acid sequences set forth in Table 2 (SEQ ID NOS: 1 to 292), Table 3 (SEQ ID NOS: 293 to 347), or both;
the immunodominant peptides are selected from 1, 2 or more peptides selected from the amino acid sequences set forth in those sequences set forth in Table 3;
the peptide or peptides include amino acid sequences set forth in Table 2 and Table 3.

140.-143. (canceled)

144. A method of selecting an immunoprevalent or immunodominant peptide or protein of a virus comprising, consisting of, or consisting essentially of:
obtaining an amino acid sequence of the virus;
determining one or more sets of overlapping peptides spanning one or more virus antigen using unbiased selection;
synthesizing one or more pools of virus peptides comprising the one or more sets of overlapping peptides;
combining the one or more pools of virus peptides with Class I major histocompatibility proteins (MHC), Class II MHC, or both Class I and Class II MHC to form peptide-MHC complexes;
contacting the peptide-MHC complexes with T cells from subjects exposed to the virus;
determining which pools triggered cytokine release by the T cells; and
deconvoluting from the pool of peptides that elicited cytokine release by the T cells, which peptide or peptides are immunoprevalent or immunodominant in the pool.

145. The method of claim 144, wherein at least one of:
the virus is a coronavirus;
the virus is SARS-CoV-2;
the immunodominant peptides are selected from 1, 2 or more peptides selected from the amino acid sequences set forth in Table 2 (SEQ ID NOS: 1 to 292), Table 3 (SEQ ID NOS: 293 to 347), or both;
the immunodominant peptides are selected from 1, 2 or more peptides selected from the amino acid sequences set forth in those sequences set forth in Table 3;
the peptide or peptides include amino acid sequences set forth in Table 2 and Table 3.

146.-149. (canceled)

150. A polynucleotide that expresses one or more peptides or proteins, comprising, consisting of, or consisting essentially of an amino acid sequence selected from those sequences set forth in Table 2 (SEQ ID NOS: 1 to 292), Table 3 (SEQ ID NOS: 293 to 347), or both, or a subsequence, portion, homologue, variant or derivative thereof,
a fusion protein comprising one or more amino acid sequences selected from those sequences set forth in Table 2, Table 3, or both; or
a pool of 2 or more peptides comprising, consisting of, or consisting essentially of amino acid sequences selected from those sequences set forth in Table 2, Table 3, or both.

151. The polynucleotide of claim 150, further comprising a vector, wherein the vector is optionally a viral vector.

152.-157. (canceled)

158. A peptide-major histocompatibility complex (MHC)/peptide multimer comprising at least one of:
two MHC/peptide monomers, wherein at least one MHC/peptide monomer comprises a peptide derived from SARS-CoV-2 B.1.1.7, SARS-CoV-2 B1.351. SARS-CoV-2 P.1. and/or SARS-CoV-2 CAL.20C,
at least one MHC/peptide monomer comprises a peptide which comprises, consists of, or consists essentially of an amino acid sequence selected from the sequences set forth in Table 2 (SEQ ID NOS: 1 to 292) and/or Table 3 (SEQ ID NOS: 293 to 347);
at least one MHC/peptide monomer comprises a peptide derived from SARS-CoV-2 Spike (S) protein such as a SARS-CoV-2 Spike (S) protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 Membrane (M) protein such as a SARS-CoV-2 Membrane (M) protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 Nucleocapsid (N) protein such as a SARS-CoV-2 Nucleocapsid (N) protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 Envelope (E) protein such as a SARS-CoV-2 Envelope (E) protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 ORF3a protein such as a SARS-CoV-2 ORF3a protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 ORF7a protein such as a SARS-CoV-2 ORF7a protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 ORF8 protein such as a SARS-CoV-2 ORF8 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp1 protein such as a SARS-CoV-2 nsp1 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp2 protein such as a SARS-CoV-2 nsp2 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp3 protein such as a SARS-CoV-2 nsp3 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp6 protein such as a SARS-CoV-2 nsp6 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp9 protein such as a SARS-CoV-2 nsp9 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp10 protein such as a SARS-CoV-2 nsp10 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp12 protein such as a SARS-CoV-2 nsp12 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp13 protein such as a SARS-CoV-2 nsp13 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp14 protein such as a SARS-CoV-2 nsp14 protein derived peptide set forth in Table 2 (SEQ ID NOS: 1 to 292) and/or Table 3 (SEQ ID NOS: 293 to 347);
at least one MHC/peptide monomer comprises a peptide derived from SARS-CoV-2 nsp15 protein such as a SARS-CoV-2 nsp15 protein derived peptide set forth in Table 2 (SEQ ID NOS: 1 to 292) and/or Table 3 (SEQ ID NOS: 293 to 347);
an amino acid sequence selected from the SARS-CoV-2 P.1. derived sequences set forth in Table 2 (SEQ ID NOS: 1 to 292) and/or Table 3 (SEQ ID NOS: 293 to 347).

159.-180. (canceled)

181. The MHC/peptide multimer of claim 158, wherein at least one of:
the at least two MHC/peptide monomers are identical,
the MHC/peptide multimer comprise at least 3, such as at least 4, 5, 6, 7, 8, 9, 10, 11, 12, 13, 14, 15, 16, 17, 18, 19 or 20 MHC/peptide monomers;
the MHC/peptide multimer comprise at least 3, such as at least 4, 5, 6, 7, 8, 9, 10, 11, 12, 13, 14, 15, 16, 17, 18, 19 or 20 identical MHC/peptide monomers;
the MHC/peptide multimer comprise at least 3, such as at least 4, 5, 6, 7, 8, 9, 10, 11, 12, 13, 14, 15, 16, 17, 18, 19 or 20 different MHC/peptide monomers;
at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the sequences set forth in Table 2 (SEQ ID NOS: 1 to 292) and/or Table 3 (SEQ ID NOS: 293 to 347);
at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 B.1.1.7 derived sequences set forth in Table 2 (SEQ ID NOS: 1 to 292) and/or Table 3 (SEQ ID NOS: 293 to 347);
at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 B.1.351 derived sequences set forth in Table 2 (SEQ ID NOS: 1 to 292) and/or Table 3 (SEQ ID NOS: 293 to 347);
at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 P.1. derived sequences set forth in Table 2 (SEQ ID NOS: 1 to 292) and/or Table 3 (SEQ ID NOS: 293 to 347);
at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 CAL.20C derived sequences set forth in Table 2 (SEQ ID NOS: 1 to 292) and/or Table 3 (SEQ ID NOS: 293 to 347); or
at least 3, such as at least 4, 5, 6, 7, 8, 9, 10, 11, 12, 13, 14, 15, 16, 17, 18, 19 or 20 of the MHC/peptide monomers comprises a peptide which comprises, consists of, or consists essentially of an amino acid sequence selected from the SARS-CoV-2 Spike (S) protein, Membrane (M) protein, Nucleocapsid (N) protein, Envelope (E) protein, ORF3a, ORF7a, ORF8, nsp1, nsp2, nsp3, nsp6, nsp9, nsp10, nsp12, nsp13, nsp14 and/or nsp15 derived sequences set forth in Table 2 (SEQ ID NOS: 1 to 292) and/or Table 3 (SEQ ID NOS: 293 to 347).

182.-190. (canceled)

191. The MHC/peptide multimer of claim 158, wherein each MHC/peptide monomer of the MHC/peptide multimer is associated with one or more multimerization domains such as a multimerization domain selected from the group consisting of proteins, peptides, albumins, immunoglobulins, coiled-coil helixes, polynucleotides, IgG, streptavidin, avidin, streptactin, micelles, cells, polymers, dextran, polysaccharides, beads and other types of solid support, and small organic molecules carrying reactive groups or carrying chemical motifs that can bind MHC/peptide monomers.

192. The MHC/peptide multimer of claim 158, wherein the multimer comprises at least one of:
no more than 30 MHC/peptide monomers in total, such as no more than 25 MHC/peptide monomers, such as no more than 20 MHC/peptide monomers, such as no more than 15 MHC/peptide monomers, or no more than 10 MHC/peptide monomers in total;
from 2 to 4 MHC/peptide monomers, such as from 4 to 6 MHC/peptide monomers, such as from 6 to 8 MHC/peptide monomers, such as from 8 to 10 MHC/peptide monomers, such as from 10 to 12 MHC/peptide monomers, such as from 12 to 14 MHC/peptide monomers, such as from 14 to 16 MHC/peptide monomers, such as from 16 to 18 MHC/peptide monomers, such as from 18 to 20 MHC/peptide monomers, such as from 20 to 25 MHC/peptide monomers, such as from 25 to 30 MHC/peptide monomers, such as from 30 to 40 MHC/peptide monomers, such as from 40 to 50 MHC/peptide monomers, such as from 10 to 20 MHC/peptide monomers or any combination of these intervals;
at least 2, 3, 4, 5, 6, 7, 8, 9, 10, 11, 12, 13, 14, 15, 16, 17, 18, 19, 20, 21, 22, 23, 24 or 25 MHC/peptide monomers or has 2, 3, 4, 5, 6, 7, 8, 9, 10, 11, 12, 13, 14, 15, 16, 17, 18, 19, 20, 21, 22, 23, 24 or 25 MHC/peptide monomers in total;
MHC Class I/peptide monomers or wherein all MHC monomers of the MHC/peptide multimer are MHC Class I/peptide monomers.
the MHC/peptide multimer comprises MHC Class II/peptide monomers or wherein all MHC/peptide monomers of the MHC/peptide multimer are MHC Class II/peptide monomers;
the MHC/peptide multimer comprises MHC Class I/peptide and MHC Class II/peptide monomers or wherein all MHC/peptide monomers of the MHC/peptide multimer are either MHC Class I/peptide monomers or MHC Class II/peptide monomers;
some of the MHC/peptide monomers or all of the MHC/peptide monomers have identical peptides;
some of the MHC/peptide monomers or all of the MHC/peptide monomers have different peptides;
at least 2, such as at least 3, 4, 5, 6, 7, 8, 9, 10, 11, 12, 13, 1, 15, 16, 17, 18, 19 or 20 of the MHC/peptide monomers comprise different peptides;
at least 2, 3, 4, 5, 6, 7, 8, 9, 10, 20, 30, 40, 50, 100 or 1000 MHC/peptide multimers; or
at least 2, 3, 4, 5, 6, 7, 8, 9, 10, 15, 20, 25, 50, 100, 500 or 1000 different MHC/peptide multimers.

193.-200. (canceled)

201. The MHC/peptide multimer of claim 158, further comprising at least one of:
one or more labels such as at least two labels;
the labels are different or at least some of the labels are different.
the labels comprise at least one fluorescent label;
the labels comprise at least one oligonucleotide label such as a nucleic acid molecule comprises or consists of DNA, RNA, and/or artificial nucleotides such as PLA or LNA;
the labels comprise at least one fluorescent label and at least one oligonucleotide label;
the label is a oligonucleotide comprising one or more of: barcode region, 5′ first primer region (forward), 3′ second primer region (reverse), random nucleotide region, connector molecule, stability-increasing components, short nucleotide linkers in between any of the above-mentioned components, adaptors for sequencing, and annealing region;
the labels comprise at least one such as one or more labels selected from the group consisting of APC, APC-Cy7, ABC-H7, APC-R700, Alexa Flours™ 488, Alexa Flours™555, Alexa Flours™647, Alexa Flours™700, AmCyan, BB151, BB700, BUV395, BUV496, BUV563, BUV615, BUV661, BUV737, BUV805, BV421, BV480, BV510, BV605, BV711, BV750, BV786, FITC, PE, PE-CF594, PE-Cy5, PE-CY5.5, PE-cy7, Pasific Blue, PERCP, pPerCp-Cy5.5, PE, R718, RY586, V450, V500, cFluor®B515, cFluor®B532, cFluor®B548, cFluor®B675, cFluor®B690, cFluor®BY575, cFluor®BY610, cFluor®BY667, cFluor®BY710, cFluor®BY750, cFluor®BY781, cFluor®B250, cFluor®R659, cFluor®R668, cFluor®R685, cFluor®R720, cFluor®R780, cFluor®R840, cFluor®v420, cFluor®v547, cFluor®v450, cFluor®v610 and cFluor®YG610;
the one or more labels is a chemiluminescent label such as a label selected from the group consisting of luminol, isoluminol, theromatic acridinium ester, imidazole, acridinium salt and oxalate ester;
the one or more labels is a bioluminescent label such as a label selected from the group consisting of luciferin, luciferase and aequorin;
the one or more labels is an enzyme label, such as an enzyme label selected from the group peroxidases, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase;
the one or more labels is a chromophore label;
the one or more labels is a metal label;
the one or more labels is a radioactive label such as a label selected from the group consisting of a radionuclide, an isotope, a label comprising α rays, a label comprising β rays or a label comprising γ rays.

202.-213. (canceled)

214. A composition comprising at least two MHC/peptide multimers of claim 158 selected from:
at least 2, 3, 4, 5, 6, 7, 8, 9, 10, 20, 30, 40, 50, 100 or 1000 MHC/peptide multimers;
at least 2, 3, 4, 5, 6, 7, 8, 9, 10, 15, 20, 25, 50, 100, 500 or 1000 different MHC/peptide multimers;
at least 2, 3, 4, 5, 6, 7, 8, 9, 10, 20, 30, 40, 50, 100 or 1000 MHC/peptide multimers of the composition are different each comprising one or more peptides selected from one or more of the following groups:
i) one or more peptides derived from SARS-CoV-2 B.1.1.7, such as one or more SARS-CoV-2 B.1.1.7 derived peptides set forth in Table 2 (SEQ ID NOS: 1 to 292) and/or Table 3 (SEQ ID NOS: 293 to 347),
ii) one or more peptides derived from SARS-CoV-2 B1.351. such as one or more SARS-CoV-2 B1.351 derived peptides set forth in Table 2 (SEQ ID NOS: 1 to 292) and/or Table 3 (SEQ ID NOS: 293 to 347)
iii) one or more peptides derived from SARS-CoV-2 P.1, such as one or more SARS-CoV-2 P.1 derived peptides set forth in Table 2 (SEQ ID NOS: 1 to 292) and/or Table 3 (SEQ ID NOS: 293 to 347).
iv) one or more peptides derived from SARS-CoV-2 CAL.20C, such as one or more SARS-CoV-2 CAL.20C derived peptides set forth in Table 2 (SEQ ID NOS: 1 to 292) and/or Table 3 (SEQ ID NOS: 293 to 347),
v) one or more peptides set forth in Table 2 (SEQ ID NOS: 1 to 292) and/or Table 3 (SEQ ID NOS: 293 to 347),
vi) one or more peptides derived from the SARS-CoV-2 Spike (S) protein such as one or more SARS-CoV-2 Spike (S) protein derived peptides set forth in Table 2 (SEQ ID NOS: 1 to 292) and/or Table 3 (SEQ ID NOS: 293 to 347),
vii) one or more peptides derived from the SARS-CoV-2 Membrane (M) protein such as one or more SARS-CoV-2 Membrane (M) protein derived peptides set forth in Table 2 (SEQ ID NOS: 1 to 292) and/or Table 3 (SEQ ID NOS: 293 to 347),
viii) one or more peptides derived from the SARS-CoV-2 Nucleocapsid (N) protein such as one or more SARS-CoV-2 Nucleocapsid (N) protein derived peptides set forth in Table 2 (SEQ ID NOS: 1 to 292) and/or Table 3 (SEQ ID NOS: 293 to 347),
ix) one or more peptides derived from the SARS-CoV-2 Envelope (E) protein such as one or more SARS-CoV-2 Envelope (E) protein derived peptides set forth in Table 2 (SEQ ID NOS: 1 to 292) and/or Table 3 (SEQ ID NOS: 293 to 347).
x) one or more peptides derived from the SARS-CoV-2 ORF3a protein such as one or more SARS-CoV-2 ORF3a protein derived peptides set forth in Table 2 (SEQ ID NOS: 1 to 292) and/or Table 3 (SEQ ID NOS: 293 to 347),
xi) one or more peptides derived from the SARS-CoV-2 ORF7a protein such as one or more SARS-CoV-2 ORF7a protein derived peptides set forth in Table 2 (SEQ ID NOS: 1 to 292) and/or Table 3 (SEQ ID NOS: 293 to 347),
xii) one or more peptides derived from the SARS-CoV-2 ORF8 protein such as one or more SARS-CoV-2 ORF8 protein derived peptides set forth in Table 2 (SEQ ID NOS: 1 to 292) and/or Table 3 (SEQ ID NOS: 293 to 347),
xiii) one or more peptides derived from the SARS-CoV-2 nsp1 protein such as one or more SARS-CoV-2 nsp1 protein derived peptides set forth in Table 2 (SEQ ID NOS: 1 to 292) and/or Table 3 (SEQ ID NOS: 293 to 347),
xiv) one or more peptides derived from the SARS-CoV-2 nsp2 protein such as one or more SARS-CoV-2 nsp2 protein derived peptides set forth in Table 2 (SEQ ID NOS: 1 to 292) and/or Table 3 (SEQ ID NOS: 293 to 347),
xv) one or more peptides derived from the SARS-CoV-2 nsp3 protein such as one or more SARS-CoV-2 nsp3 protein derived peptides set forth in Table 2 (SEQ ID NOS: 1 to 292) and/or Table 3 (SEQ ID NOS: 293 to 347),
xvi) one or more peptides derived from the SARS-CoV-2 nsp6 protein such as one or more SARS-CoV-2 nsp6 protein derived peptides set forth in Table 2 (SEQ ID NOS: 1 to 292) and/or Table 3 (SEQ ID NOS: 293 to 347),
xvii) one or more peptides derived from the SARS-CoV-2 nsp9 protein such as one or more SARS-CoV-2 nsp9 protein derived peptides set forth in Table 2 (SEQ ID NOS: 1 to 292) and/or Table 3 (SEQ ID NOS: 293 to 347),
xviii) one or more peptides derived from the SARS-CoV-2 nsp10 protein such as one or more SARS-CoV-2 nsp10 protein derived peptide set forth in Table 2 (SEQ ID NOS: 1 to 292) and/or Table 3 (SEQ ID NOS: 293 to 347),
xix) one or more peptides derived from the SARS-CoV-2 nsp12 protein such as one or more SARS-CoV-2 nsp12 protein derived peptides set forth in Table 2 (SEQ ID NOS: 1 to 292) and/or Table 3 (SEQ ID NOS: 293 to 347),
xx) one or more peptides derived from the SARS-CoV-2 nsp13 protein such as one or more SARS-CoV-2 nsp13 protein derived peptides set forth in Table 2 (SEQ ID NOS: 1 to 292) and/or Table 3 (SEQ ID NOS: 293 to 347),
xxi) one or more peptides derived from the SARS-CoV-2 nsp14 protein such as one or more SARS-CoV-2 nsp14 protein derived peptides set forth in Table 2 (SEQ ID NOS: 1 to 292) and/or Table 3 (SEQ ID NOS: 293 to 347) and
xxii) one or more peptides derived from the SARS-CoV-2 nsp15 protein such as one or more SARS-CoV-2 nsp15 protein derived peptides set forth in Table 2 (SEQ ID NOS: 1 to 292) and/or Table 3 (SEQ ID NOS: 293 to 347).

215.-216. (canceled)

217. A method for monitoring, diagnosing, isolating, or detecting an immune response relevant to a coronavirus infection comprising one or more steps of:
i) providing one or more MHC/peptide multimers of claim 158,
ii) providing a sample comprising a population of T cells, and
iii) measuring the presence, frequency, number, activity and/or state of T cells
specific for said one or more MHC/peptide multimers,
thereby monitoring, diagnosing, isolating, or detecting said immune response relevant to a coronavirus infection.

218. (canceled)

219. (canceled)

220. (canceled)
